(19) United States
(12) Patent Application Publication
(10) Pub. No.: US 2010/0062505 A1
(43) Pub. Date: Mar. 11, 2010

(54) BUTANOL PRODUCTION BY METABOLICALLY ENGINEERED YEAST

(75) Inventors: Uvini Gunawardena, Pasadena, CA (US); Peter Meinhold, Pasadena, CA (US); Matthew W. Peters, Pasadena, CA (US); Jun Urano, Culver City, CA (US); Reid M. Renny Feldman, San Marino, CA (US)

Correspondence Address: COOLEY GODWARD KRONISH LLP;ATTN: Patent Group, Suite 1100, 777 - 6th Street, NW, WASHINGTON, DC, 20001, US

(73) Assignee: Gevo, Inc., Pasadena, CA (US)

(21) Appl. No.: 11/963,542

(22) Filed: Dec. 21, 2007

Related U.S. Application Data
(60) Provisional application No. 60/871,427, filed on Dec. 21, 2006.
(60) Provisional application No. 60/888,016, filed on Feb. 2, 2007.
(60) Provisional application No. 60/928,283, filed on May 8, 2007.

(51) Int. Cl. C12P 7/16; C12N 1/18; C12N 1/15

(52) U.S. Cl. 435/160; 435/254.2; 435/254.21

(57) ABSTRACT

There are disclosed metabolically-engineered yeast and methods of producing n-butanol. In an embodiment, metabolically-engineered yeast is capable of metabolizing a carbon source to produce n-butanol, at least one pathway produces increased cytosolic acetyl-CoA relative to cytosolic acetyl-CoA produced by a wild-type yeast, and at least one heterologous gene encodes and expresses at least one enzyme for a metabolic pathway capable of utilizing NADH to convert acetyl-CoA to n-butanol. In another embodiment, a method of producing n-butanol includes (a) providing metabolically-engineered yeast capable of metabolizing a carbon source to produce n-butanol, at least one pathway produces increased cytosolic acetyl-CoA relative to cytosolic acetyl-CoA produced by a wild-type yeast, and at least one heterologous gene encodes and expresses at least one enzyme for a metabolic pathway utilizing NADH to convert acetyl-CoA to n-butanol; and (b) culturing the yeast to produce n-butanol. Other embodiments are also disclosed.

This application claims the benefit of (1) U.S. Provisional Patent Application Ser. No. 60/871,427, filed Dec. 21, 2006, by Jun Urano, et al., for BUTANOL PRODUCTION BY METABOLICALLY ENGINEERED YEAST; (2) U.S. Provisional Patent Application Ser. No. 60/888,016, filed Feb. 2, 2007, by Jun Urano, et al., for N-BUTANOL PRODUCTION BY METABOLICALLY ENGINEERED YEAST; and (3) U.S. Provisional Patent Application Ser. No. 60/928,283, filed May 8, 2007, by Uvini P. Gunawardena, et al., for BUTANOL PRODUCTION BY METABOLICALLY ENGINEERED YEAST. Each of the above-identified applications are hereby incorporated herein by reference.

### FIELD OF THE INVENTION

The present invention relates to metabolically engineering yeast cells for the production of n-butanol at high yield as an alternative and renewable transportation fuel, and for other applications. The yeasts of the invention are engineered to comprise a metabolic pathway that converts a carbon source such as glucose and/or other metabolizable carbohydrates, as well as biomass and the like, to n-butanol.

### BACKGROUND

Currently, approximately 140 billion gallons of gasoline are consumed in the United States and approximately 340 billion gallons are consumed worldwide per year. These quantities of consumption are only growing. The Energy Policy Act of 2005 stipulates that 7.5 billion gallons of renewable fuels be used in gasoline by 2012. In his 2007 State of the Union address, the President called for increasing the size and expanding the scope of renewable fuel standard (RFS) to require 35 billion gallons of renewable and alternative fuels in 2017. The Department of Energy has set a goal of replacing 30 percent of the United States' current gasoline consumption with biofuels by 2030 (the “30×30” initiative). In March 2007, Brazil and the United States signed “the Ethanol Agreement,” to promote the development of biofuels in the Americas, uniting the largest biofuel producers in the world—currently accounting for 70 percent of the world's ethanol production.

Biofuels have the potential to not only reduce the United States' dependency on foreign oil imports, which is vital to homeland security, but to also dramatically decrease greenhouse gas emissions associated with global warming. Biofuels can be obtained from the conversion of carbon based feedstock. Agricultural feedstocks are considered renewable because, although they release carbon dioxide when burned, they capture nearly an equivalent amount of carbon dioxide through photosynthesis.

In the United States, ethanol is increasingly being used as an oxygenate additive for standard gasoline, as a replacement for methyl t-butyl ether (MTBE), the latter chemical being difficult to retrieve from groundwater and soil contamination. At a 10% mixture, ethanol reduces the likelihood of engine knock, by raising the octane rating. The use of 10% ethanol gasoline is mandated in some cities where the possibility of harmful levels of auto emissions are possible, especially during the winter months. North American vehicles from approximately 1980 onward can run on 10% ethanol/90% gasoline (i.e., E10) with no modifications.

In order for ethanol to be used at higher concentrations, however, a vehicle must have its engine and fuel system specially engineered or modified. Flexible fuel vehicles (FFVs), are designed to run on gasoline or a blend of up to 85% ethanol (E85). However, since a gallon of ethanol contains less energy than a gallon of gasoline, FFVs typically get about 20-30% fewer miles per gallon when fueled with E85. Conversion packages are available to convert a conventional vehicle to a FFV that typically include an electronic device to increase injected fuel volume per cycle (because of the lower energy content of ethanol) and, in some cases, a chemical treatment to protect the engine from corrosion. Over 4 million flexible-fuel vehicles are currently operated on the road in the United States, although a 2002 study found that less than 1% of fuel consumed by these vehicles is E85.

Butanol has several advantages over ethanol for fuel. While it can be made from the same feedstocks as ethanol, unlike ethanol, it is compatible with gasoline and petrodiesel at any ratio. Butanol can also be used as a pure fuel in existing cars without modifications and has been proposed as a jet fuel by the Sir Richard Branson Group at Virgin Airlines. Unlike ethanol, butanol does not absorb water and can thus be stored and distributed in the existing petrochemical infrastructure. Due to its higher energy content, the fuel economy (miles per gallon) is better than that of ethanol. Also, butanol-gasoline blends have lower vapor pressure than ethanol-gasoline blends, which is important in reducing evaporative hydrocarbon emissions. These properties provide the potential for butanol to be used in precisely the same manner as gasoline, without vehicle modification and without the burden on consumers of having to refuel more often.

n-Butanol can be produced using Clostridium strains that naturally produce n-butanol via a pathway that leads from butyryl-CoA to n-butanol. One disadvantage of Clostridium strains is that n-butanol production occurs in a two-step process that involves an acid-producing growth phase followed by a solvent production phase. Also, large quantities of byproducts, such as hydrogen, ethanol, and acetone are produced in this process, thus limiting the stoichiometric yield of n-butanol to about 0.6 mol of n-butanol per mol of glucose consumed. Further, Clostridium strains lose their ability to produce solvents under continuous culture conditions (Cornillot et al., J. Bacteria 179: 5442-5447, 1997). The Clostridium pathway showing the conversion of glucose to acids and solvents in C. acetobutylicum, including the path to produce n-butanol from acetyl-CoA, is shown in FIG. 1.

### SUMMARY OF THE INVENTION

In an embodiment, there is provided a metabolically-engineered yeast capable of metabolizing a carbon source to produce n-butanol, at least one pathway configured for producing an increased amount of cytosolic acetyl-CoA relative to another amount of cytosolic acetyl-CoA produced by a wild-type yeast, and at least one heterologous gene to encode and express at least one enzyme for a metabolic pathway capable of utilizing NADH to convert acetyl-CoA to the n-butanol.

In another embodiment, there is provided a method of producing n-butanol, the method comprising (a) providing metabolically-engineered yeast capable of metabolizing a carbon source to produce n-butanol, at least one pathway configured for producing an increased amount of cytosolic acetyl-CoA relative to another amount of cytosolic acetyl-CoA produced by a wild-type yeast, and at least one heterologous gene to encode and express at least one enzyme for a metabolic pathway capable of utilizing NADH to convert acetyl-CoA to the n-butanol; and (b) culturing the metabolically-engineered yeast for a period of time and under conditions to produce the n-butanol.

In yet another embodiment, there is provided a method of producing n butanol, using yeast, the method comprising (a) metabolically engineering the yeast to increase cytosolic acetyl-CoA production; (b) metabolically engineering the yeast to express a metabolic pathway that converts a carbon source to n butanol, wherein the pathway requires at least one non-native enzyme of the yeast, wherein steps (a) and (b) can be performed in either order; and (c) culturing the yeast for a period of time and under conditions to produce a recoverable amount of n butanol.

In still another embodiment, there is provided a method of producing n butanol, using yeast, the method comprising (a) culturing a metabolically-engineered yeast for a period of time and under conditions to produce a yeast-cell biomass without activating n butanol production; and (b) altering the culture conditions for another period of time and under conditions to produce a recoverable amount of n butanol

In another embodiment, there is provided a metabolically-engineered yeast capable of metabolizing a carbon source and producing an increased amount of acetyl-CoA relative to the amount of cytosolic acetyl-CoA produced by a wild-type yeast.

In yet another embodiment, there is provided a method of increasing metabolic activity of yeast, the method comprising producing an increased amount of cytosolic acetyl-CoA of the yeast relative to another amount of cytosolic acetyl-CoA produced by a wild-type yeast.

In still another embodiment, there is provided a metabolically-engineered yeast having at least one pathway configured for producing an increased amount of cytosolic acetyl-CoA relative to another amount of cytosolic acetyl-CoA produced by a wild-type yeast.

Other embodiments are also disclosed.

### BRIEF DESCRIPTION OF THE DRAWINGS

Illustrative embodiments of the invention are illustrated in the drawings, in which:

FIG. 1 illustrates the metabolic pathways involved in the conversion of glucose, pentose, and granulose to acids and solvents in Clostridium acetobutylicum. Hexoses (e.g., glucose) and pentoses are converted to pyruvate, ATP and NADH. Subsequently, pyruvate is oxidatively decarboxylated to acetyl-CoA by a pyruvate-ferredoxin oxidoreductase. The reducing equivalents generated in this step are converted to hydrogen by an iron-only hydrogenase. Acetyl-CoA is the branch-point intermediate, leading to the production of organic acids (acetate and butyrate) and solvents (acetone, butanol and ethanol.

FIG. 2 illustrates a chemical pathway to produce butanol in yeasts.

FIG. 3 illustrates pathways used by Saccharomyces cerevisiae to generate acetyl-CoA.

FIGS. 4 and 5 illustrate various exemplary plasmids that may be used to express various enzymes in accordance with the present disclosure.

FIG. 4 illustrates an exemplary plasmid that may be used to express various enzymes in accordance with the present disclosure as described in Table 1.

FIG. 5 an exemplary plasmid that may be used to express various enzymes in accordance with the present disclosure as described in Table 2.

FIG. 6 graphically illustrates n-butanol production over time by Gevo 1099 and Gevo 1103 as compared to the Vector only control isolates, Gevo 1110 and Gevo 1111, as follows:

( [symbol] ) Gevo 1099;

( [symbol] ) Gevo 1103;

( [symbol] ) Gevo 1110; and

( [symbol] ) Gevo 1111.

FIG. 7 illustrates the pGV1090 plasmid containing bcd, etfb, and etfa genes from C. acetobutylicum inserted at the EcoRI and BamHI sites and downstream from a modified phage lambda LacO-1 promoter (P_{L-lac}). The plasmid also carries a replication origin gene of pBR322 and a chloramphenicol resistance gene.

FIG. 8 illustrates the pGV1095 plasmid for expression of butyraldehyde dehydrogenase (bdhB) from C. acetobutylicum inserted at the EcoRI and BamHI sites and downstream from a modified phage lambda LacO-1 promoter (P_{L-lac}). The plasmid also carries a replication origin gene of ColE1 and a chloramphenyicol resistance gene.

FIG. 9 illustrates the pGV1094 plasmid for expression of crotonase (crt) from C. acetobutylicum inserted at the EcoRI and BamHI sites and downstream from a modified phage lambda LacO-1 promoter (P_{L-lac}). The plasmid also carries an on gene and a chloramphenyicol resistance gene.

FIG. 10 illustrates the pGV1037 plasmid for expression of hydroxybutyryl-CoA dehydrogenase (hbd) from C. acetobutylicum inserted at the EcoRI and BamHI sites and downstream from a modified phage lambda LacO-1 promoter (P_{L-lac}). The plasmid also carries an on gene and a chloramphenicol resistance gene.

FIG. 11 illustrates the pGV1031 plasmid for expression of thiolase (thl) from C. acetobutylicum inserted at the EcoRI and BamHI sites and downstream from a LacZ gene. The plasmid also carries a replication origin gene of pBR322 and an a ampicillin resistance gene.

FIG. 12 illustrates the pGV1049 plasmid for expression of crotonase from Clostridium beijerinckii inserted at the EcoRI and BamHI sites and downstream from a modified phage lambda LacO-1 promoter (P_{L-lac}). The plasmid also carries an ori gene and a chloramphenicol resistance gene.

FIG. 13 illustrates the pGV1050 plasmid for expression of hydroxybutyryl-CoA dehydrogenase (hbd) from C. beijerinckii inserted at the EcoRI and BamHI sites and downstream from a modified phage lambda LacO-1 promoter (P_{L-lac}). The plasmid also carries an ori gene and a chloramphenicol resistance gene.

FIG. 14 illustrates the pGV1091 plasmid for expression of alcohol dehydrogenase (adhA) from C. beijerinckii inserted at the HindIII and BamHI sites and downstream from a modified phage lambda LacO-1 promoter (P_{L-lac}). The plasmid also carries a chloramphenicol resistance gene.

FIG. 15 illustrates the pGV1096 plasmid for expression of alcohol dehydrogenase (aldh) from C. beijerinckii inserted at the EcoRI and BamHI sites and downstream from a modified phage lambda LacO-1 promoter (P_{L-lac}). The plasmid also carries an ori gene and a chloramphenicol resistance gene.

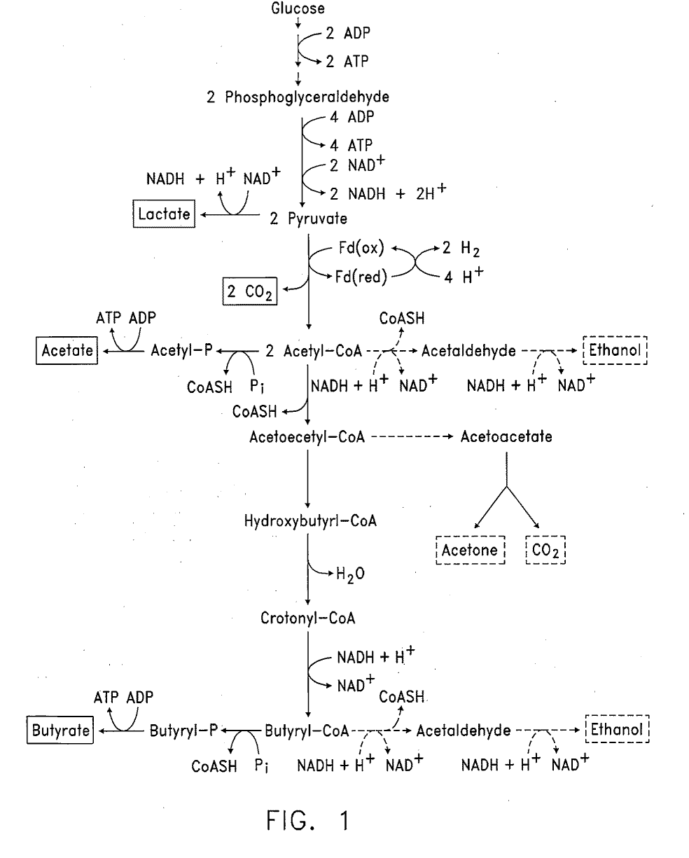

### DETAILED DESCRIPTION

Recombinant yeast microorganisms are described that are engineered to convert a carbon source into n-butanol at high yield. In particular, recombinant yeast microorganisms are described that are capable of metabolizing a carbon source for producing n-butanol at a yield of at least 5% of theoretical, and, in some cases, a yield of over 50% of theoretical. As used herein, the term “yield” refers to the molar yield. For example, the yield equals 100% when one mole of glucose is converted to one mole of n-butanol. In particular, the term “yield” is defined as the mole of product obtained per mole of carbon source monomer and may be expressed as percent. Unless otherwise noted, yield is expressed as a percentage of the theoretical yield. “Theoretical yield” is defined as the maximum moles of product that can be generated per a given mole of substrate as dictated by the stoichiometry of the metabolic pathway used to make the product. For example, the theoretical yield for one typical conversion of glucose to n-butanol is 100%. As such, a yield of n-butanol from glucose of 95% would be expressed as 95% of theoretical or 95% theoretical yield.

The microorganisms herein disclosed are engineered, using genetic engineering techniques, to provide microorganisms which utilize heterologously expressed enzymes to produce n-butanol at high yield. Butanol yield is dependent on the high-yield conversion of a carbon source to acetyl-CoA, and the subsequent high-yield conversion of acetyl-CoA to butanol. The invention relates to the combination of these two aspects resulting in a microorganism that produces n-butanol at a high yield.

As used herein, the term “microorganism” includes prokaryotic and eukaryotic microbial species from the Domains Bacteria and Eukaryote, the latter including yeast and filamentous fungi, protozoa, algae, or higher Protista. The terms “cell,” “microbial cells,” and “microbes” are used interchangeably with the term microorganism. In a preferred embodiment, the microorganism is a yeast, for example, Saccharomyces cerevisiae or Kluyveromyce lactis) or E. coli.

“Yeast”, refers to a domain of eukaryotic organisms, phylogenetically placed in the kingdom fungi, under the phyla Ascomycota and Basidiomycota. Approximately 1500 yeast species are described to date. Yeasts are primarily unicellular microorganisms that reproduce primarily by asexual budding even though some multicellular yeasts and those that reproduce by binary fission are described. Most species are classified as aerobes but facultative and anaerobic yeasts are also well known. Related to yeast fermentative physiology, yeasts are categorized into two groups—Crabtree—positive and Crabtree—negative.

Briefly, the Crabtree effect is defined as the inhibition of oxygen consumption by a microorganism when cultured under aerobic conditions due to the presence of a high glucose concentration (e.g., 50 grams of glucose/L). Thus, a yeast cell having a Crabtree-positive phenotype continues to ferment irrespective of oxygen availability due to the presence of glucose, while a yeast cell having a Crabtree-negative phenotype does not exhibit glucose mediated inhibition of oxygen consumption. Examples of yeast cells typically having a Crabtree-positive phenotype include, without limitation, yeast cells of the genera Saccharomyces, Zygosaccharomyces, Torulaspora and Dekkera. Examples of yeast cells typically having a Crabtree-negative phenotype include, without limitation, yeast cells of the genera Kluyveromyces, Pichis, Hansenula and Candida.

Certain detailed aspects and embodiments of the invention are illustrated below, following a definition of certain terms used in the application. The term “carbon source” generally refers to a substrate or compound suitable to be used as a source of carbon for yeast cell growth. Carbon sources may be in various forms, including, but not limited to polymers such as xylan and pectin, carbohydrates, acids, alcohols, aldehydes, ketones, amino acids, peptides, etc. Such carbons sources more specifically include, for example, various monosaccharides such as glucose and fructose, oligosaccharides such as lactose or sucrose, polysaccharides, cellulosic material, saturated or unsaturated fatty acids, succinate, lactate, acetate, ethanol, or mixtures thereof and unpurified mixtures from renewable feedstocks, such as cheese whey permeate, cornsteep liquor, sugar beet molasses, and barley malt.

Carbon sources which serve as suitable starting materials for the production of n-butanol products include, but are not limited to, biomass hydrolysates, glucose, starch, cellulose, hemicellulose, xylose, lignin, dextrose, fructose, galactose, corn, liquefied corn meal, corn steep liquor (a byproduct of corn wet milling process that contains nutrients leached out of corn during soaking), molasses, lignocellulose, and maltose. Photosynthetic organisms can additionally produce a carbon source as a product of photosynthesis. In a preferred embodiment, carbon sources may be selected from biomass hydrolysates and glucose. Glucose, dextrose and starch can be from an endogenous or exogenous source.

It should be noted that other, more accessible and/or inexpensive carbon sources, can be substituted for glucose with relatively minor modifications to the host microorganisms. For example, in certain embodiments, use of other renewable and economically feasible substrates may be preferred. These include: agricultural waste, starch-based packaging materials, corn fiber hydrolysate, soy molasses, fruit processing industry waste, and whey permeate, etc.

Five carbon sugars are only used as carbon sources with microorganism strains that are capable of processing these sugars, for example E. coli B. In some embodiments, glycerol, a three carbon carbohydrate, may be used as a carbon source for the biotransformations. In other embodiments, glycerin, or impure glycerol obtained by the hydrolysis of triglycerides from plant and animal fats and oils, may be used as a carbon source, as long as any impurities do not adversely affect the host microorganisms.

The term “enzyme” as used herein refers to any substance that catalyzes or promotes one or more chemical or biochemical reactions, which usually includes enzymes totally or partially composed of a polypeptide, but can include enzymes composed of a different molecule including polynucleotides.

The term “polynucleotide” is used herein interchangeably with the term “nucleic acid” and refers to an organic polymer composed of two or more monomers including nucleotides, nucleosides or analogs thereof, including but not limited to single stranded or double stranded, sense or antisense deoxyribonucleic acid (DNA) of any length and, where appropriate, single stranded or double stranded, sense or antisense ribonucleic acid (RNA) of any length, including siRNA. The term “nucleotide” refers to any of several compounds that consist of a ribose or deoxyribose sugar joined to a purine or a pyrimidine base and to a phosphate group, and that are the basic structural units of nucleic acids. The term “nucleoside” refers to a compound (as guanosine or adenosine) that consists of a purine or pyrimidine base combined with deoxyribose or ribose and is found especially in nucleic acids. The term “nucleotide analog” or “nucleoside analog” refers, respectively, to a nucleotide or nucleoside in which one or more individual atoms have been replaced with a different atom or with a different functional group. Accordingly, the term polynucleotide includes nucleic acids of any length, DNA, RNA, analogs and fragments thereof. A polynucleotide of three or more nucleotides is also called nucleotidic oligomer or oligonucleotide.

The term “protein” or “polypeptide” as used herein indicates an organic polymer composed of two or more amino acidic monomers and/or analogs thereof. As used herein, the term “amino acid” or “amino acidic monomer” refers to any natural and/or synthetic amino acids including glycine and both D or L optical isomers. The term “amino acid analog” refers to an amino acid in which one or more individual atoms have been replaced, either with a different atom, or with a different functional group. Accordingly, the term polypeptide includes amino acidic polymer of any length including full length proteins, and peptides as well as analogs and fragments thereof. A polypeptide of three or more amino acids is also called a protein oligomer or oligopeptide.

The term “heterologous” or “exogenous” as used herein with reference to molecules and in particular enzymes and polynucleotides, indicates molecules that are expressed in an organism, other than the organism from which they originated or are found in nature, independently on the level of expression that can be lower, equal or higher than the level of expression of the molecule in the native microorganism.

On the other hand, the term “native” or “endogenous” as used herein with reference to molecules, and in particular enzymes and polynucleotides, indicates molecules that are expressed in the organism in which they originated or are found in nature, independently on the level of expression that can be lower, equal or higher than the level of expression of the molecule in the native microorganism.

In certain embodiments, the native, unengineered microorganism is incapable of converting a carbon source to n-butanol, or one or more of the metabolic intermediate(s) thereof, because, for example, such wild-type host lacks one or more required enzymes in a n-butanol-producing pathway.

In certain embodiments, the native, unengineered microorganism is capable of only converting minute amounts of a carbon source to n-butanol, at a yield of smaller than 0.1% of theoretical.

For instance, microorganisms such as E. coli or Saccharomyces sp. generally do not have a metabolic pathway to convert sugars such as glucose into n-butanol but it is possible to transfer a n-butanol producing pathway from a n-butanol producing strain, (e.g., Clostridium) into a bacterial or eukaryotic heterologous host, such as E. coli or Saccharomyces sp., and use the resulting recombinant microorganism to produce n-butanol.

Microorganisms, in general, are suitable as hosts if they possess inherent properties such as solvent resistance which will allow them to metabolize a carbon source in solvent containing environments.

The terms “host”, “host cells” and “recombinant host cells” are used interchangeably herein and refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

Useful hosts for producing n-butanol may be either eukaryotic or prokaryotic microorganisms. A yeast cell is the preferred host such as, but not limited to, Saccharomyces cerevisiae or Kluyveromyces lactis. In certain embodiments, other suitable yeast host microorganisms include, but are not limited to, Pichia, Yarrowia, Aspergillus, Kluyveromyces, Pachysolen, Rhodotorula, Zygosaccharomyces, Galactomyces, Schizosaccharomyces, Penicillium, Torulaspora, Debaryomyces, Williopsis, Dekkera, Kloeckera, Metschnikowia and Candida species.

In particular, the recombinant microorganisms herein disclosed are engineered to activate, and in particular express heterologous enzymes that can be used in the production of n-butanol. In particular, in certain embodiments, the recombinant microorganisms are engineered to activate heterologous enzymes that catalyze the conversion of acetyl-CoA to n-butanol.

The terms “activate” or “activation” as used herein with reference to a biologically active molecule, such as an enzyme, indicates any modification in the genome and/or proteome of a microorganism that increases the biological activity of the biologically active molecule in the microorganism. Exemplary activations include but, are not limited, to modifications that result in the conversion of the molecule from a biologically inactive form to a biologically active form and from a biologically active form to a biologically more active form, and modifications that result in the expression of the biologically active molecule in a microorganism wherein the biologically active molecule was previously not expressed. For example, activation of a biologically active molecule can be performed by expressing a native or heterologous polynucleotide encoding for the biologically active molecule in the microorganism, by expressing a native or heterologous polynucleotide encoding for an enzyme involved in the pathway for the synthesis of the biological active molecule in the microorganism, by expressing a native or heterologous molecule that enhances the expression of the biologically active molecule in the microorganism.

A gene or DNA sequence is “heterologous” to a microorganism if it is not part of the genome of that microorganism as it normally exists, i.e., it is not naturally part of the genome of the wild-type version microorganism. By way of example, and without limitation, for S. cerevisiae, a DNA encoding any one of the following is considered to be heterologous. Escherichia coli protein or enzyme, proteins or enzymes from any other microorganisms other than S. cerevisiae, non-transcriptional and translational control sequences, and a mutant or otherwise modified S. cerevisiae protein or RNA, whether the mutant arises by selection or is engineered into S. cerevisiae. Furthermore, constructs that have a wild-type S. cerevisiae protein under the transcriptional and/or translational control of a heterologous regulatory element (inducible promoter, enhancer, etc.) is also considered to be heterologous DNA.

Metabolization of a carbon source is said to be “balanced” when the NADH produced during the oxidation reactions of the carbon source equal the NADH utilized to convert acetyl-CoA to metabolization end products. Only under these conditions is all the NADH recycled. Without recycling, the NADH/NAD+ ratio becomes imbalanced (i.e. increases) which can lead the organism to ultimately die unless alternate metabolic pathways are available to maintain a balanced NADH/NAD+ ratio.

In certain embodiments, the n-butanol yield is highest if the microorganism does not use aerobic or anaerobic respiration since carbon is lost in the form of carbon dioxide in these cases.

In certain embodiments, the microorganism produces n-butanol fermentatively under anaerobic conditions so that carbon is not lost in form of carbon dioxide.

The term “aerobic respiration” refers to a respiratory pathway in which oxygen is the final electron acceptor and the energy is typically produced in the form of an ATP molecule. The term “aerobic respiratory pathway” is used herein interchangeably with the wording “aerobic metabolism”, “oxidative metabolism” or “cell respiration”.

On the other hand, the term “anaerobic respiration” refers to a respiratory pathway in which oxygen is not the final electron acceptor and the energy is typically produced in the form of an ATP molecule. This includes a respiratory pathway in which an organic or inorganic molecule other than oxygen (e.g. nitrate, fumarate, dimethylsulfoxide, sulfur compounds such as sulfate, and metal oxides) is the final electron acceptor. The wording “anaerobic respiratory pathway” is used herein interchangeably with the wording “anaerobic metabolism” and “anaerobic respiration”.

“Anaerobic respiration” has to be distinguished by “fermentation.” In “fermentation”, NADH donates its electrons to a molecule produced by the same metabolic pathway that produced the electrons carried in NADH. For example, in one of the fermentative pathways of E. coli, NADH generated through glycolysis transfers its electrons to pyruvate, yielding lactate.

A microorganism operating under fermentative conditions can only metabolize a carbon source if the fermentation is “balanced.” A fermentation is said to be “balanced” when the NADH produced during the oxidation reactions of the carbon source equal the NADH utilized to convert acetyl-CoA to fermentation end products. Only under these conditions is all the NADH recycled. Without recycling, the NADH/NAD⁺ ratio becomes imbalanced which leads the organism to ultimately die unless alternate metabolic pathways are available to maintain a balance NADH/NAD⁺ ratio. A written fermentation is said to be ‘balanced’ when the hydrogens produced during the oxidations equal the hydrogens transferred to the fermentation end products. Only under these conditions is all the NADH and reduced ferredoxin recycled to oxidized forms. It is important to know whether a fermentation is balanced, because if it is not, then the overall written reaction is incorrect.

Anaerobic conditions are preferred for a high yield n-butanol producing microorganisms.

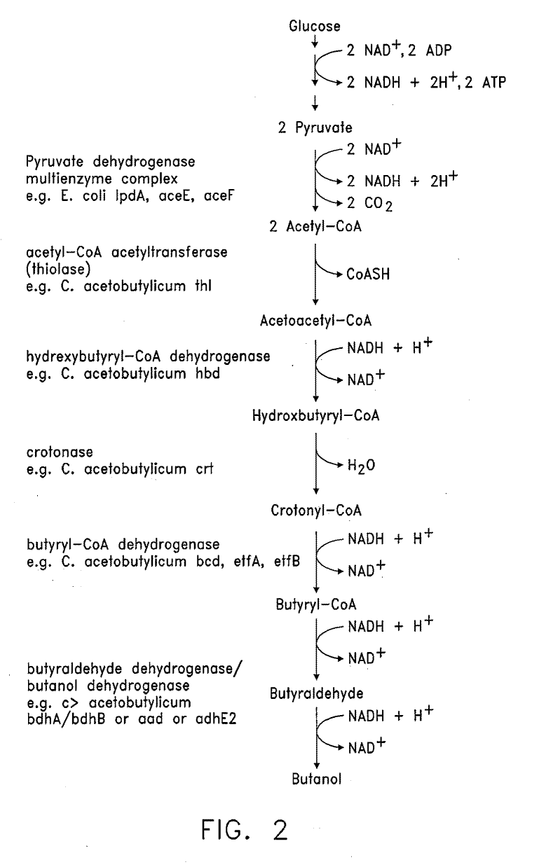

FIG. 2 illustrates a pathway in yeast that converts a carbon source to n-butanol according to an embodiment of the present invention. This pathway can be regarded as having two distinct parts, which include (1) conversion of a carbon source to acetyl-CoA, and (2) conversion of acetyl-CoA to n-butanol. Due to the compartmentalization of metabolic reactions in yeasts (and other eukaryotes) and to ensure adequate acetyl-CoA generation from glucose to drive the second part of the pathway, the production of acetyl-CoA in the cytosol is necessary and, therefore, increased in certain engineered variants disclosed herein.

Relevant to part (1) of the conversion of a carbon source to butanol, a yeast microorganism may be engineered to increase the flux of pyruvate to acetyl-CoA in the cytosol.

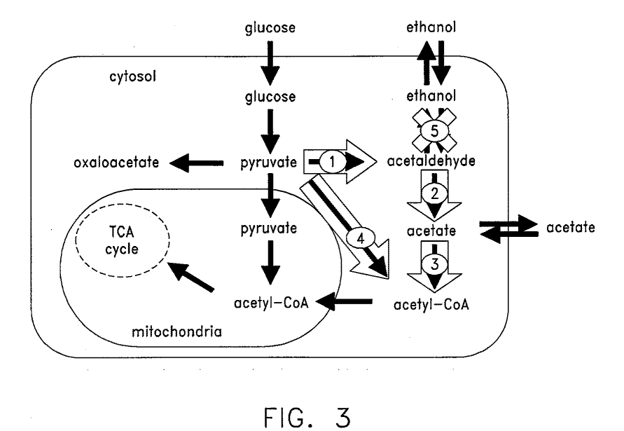

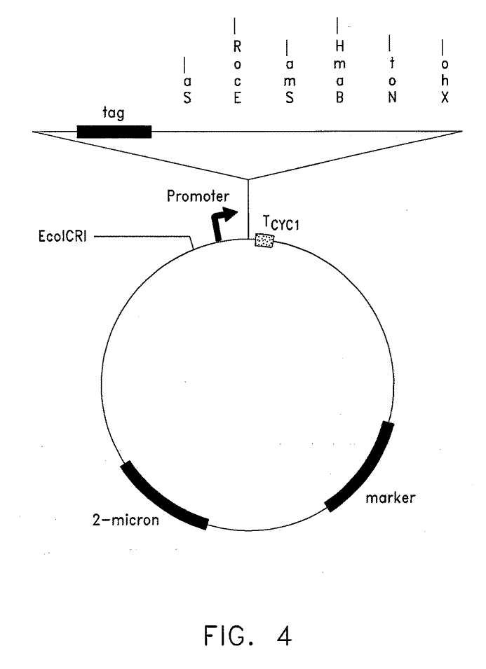

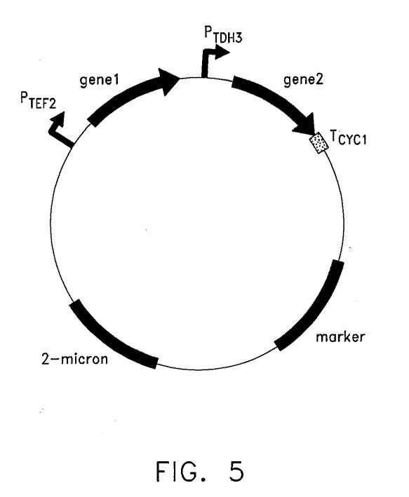

As shown in FIG. 3, S. cerevisiae generates acetyl-CoA in the mitochondria and in the cytosol. Since the conversion of acetyl-CoA to n-butanol takes part in the cytosol, the generation of acetyl-CoA in the cytosol is increased in the engineered cell. Optionally, the generation of acetyl-CoA in the mitochondrion can be reduced or repressed.

In one embodiment, acetyl-CoA may be generated from pyruvate by increasing the flux through the cytosolic “pyruvate dehydrogenase bypass” (Pronk et al., (1996). Yeast 12(16):1607), as illustrated in FIG. 3, Steps 1-3. To increase the flux through this route, one or more of the enzymes pyruvate decarboxylase (PDC), aldehyde dehydrogenase (ALD), and acetyl-CoA synthase (ACS) may be overexpressed.

This manipulation of increasing the activity or the flux of the “PDH bypass” route, can result in achieving a butanol yield of more than 5% of the theoretical maximum.

Since this route of acetyl-CoA production generates acetaldehyde as an intermediate, it is preferable to minimize diversion of acetaldehye into pathways away from acetyl-CoA synthesis, chiefly the further reduction of acetaldehyde to ethanol by the activity of alcohol dehydrogenase (ADH) enzymes. Therefore, reducing or eliminating ADH activity may further increase acetyl-CoA generation by the pyruvate dehydrogenase bypass pathway.

As an example, the genome of the Crabtree positive yeast Saccharomyces cerevisiae contains 7 known ADH genes. Of these, ADH1 is the predominant source of cytosolic ADH activity, and cells deleted for ADH1 are unable to grow anaerobically (Drewke et al., (1990). J. Bacteriology 172(7):3909) Thus, ADH1 may be preferably deleted to minimize conversion of acetaldehyde to ethanol. However, other ADH isoforms may catalyze the reduction of acetaldehyde to ethanol, and we contemplate their reduction or deletion as well.

This manipulation of decreasing the acetaldehyde conversion to ethanol, independently or in combination with the above described “PDH bypass” flux increase can result in achieving a butanol yield of more than 10% of theoretical maximum.

In addition, pyruvate dehydrogenase catalyzes the direct conversion of pyruvate to acetyl-CoA and CO₂, while reducing NAD⁺ to NADH. Thus, in certain embodiments, a pyruvate dehdyrogenase is overexpressed in the yeast cytosol. Alternatively, pyruvate is converted to formate and acetyl-CoA, and the resulting formate is further metabolized to CO₂ by the activity of formate dehydrogenase, which also reduces NAD⁺ to NADH.

Since the aforementioned routes of acetyl-CoA production utilize pyruvate as a substrate, it is preferable to minimize diversion of pyruvate in to other metabolic pathways. Pyruvate decarboxylase (PDC) activity represents a major cytoplasmic route of pyruvate metabolism. Therefore, reducing or eliminating PDC activity may further increase acetyl-CoA generation by the aforementioned routes.

The manipulation of metabolic pathways to convert pyruvate to acetyl-CoA, in combination with the elimination of the PDC activity (thus eliminating the “PDH bypass” route) may achieve a butanol yield of more than 50% of theoretical maximum. This improvement is the result of three important manipulations of the native metabolic pathways of the yeast cells: (1) eliminating carbon loss via ethanol production; (2) eliminating an energetically costly acetyl-CoA synthetase activity in the cells; and (3) by balancing the generation and consumption of co-factors (e.g. NAD+/NADH) for the entire pathway involved in the conversion of glucose to butanol (4 NADH produced from glucose to acetyl-CoA and 4 NADH consumed by the acetyl-CoA to butanol conversion). The latter two manipulations will mostly contribute to yield increase by increasing the overall metabolic fitness of a host yeast cells, thereby facilitating butanol pathway function by making ATP available for biosynthetic processes and reducing the imbalance of NAD+/NADH ratio in the cell.

Relevant to part (2) of converting a carbon source to butanol, a yeast may be engineered to convert acetyl-CoA to butanol.

In one embodiment illustrated, acetyl-CoA is converted to acetoacetyl-CoA by acetyl-CoA-acetyltransferase, acetoacetyl-CoA is converted to hydroxybutyryl-CoA by hydroxybutyryl-CoA dehydrogenase, hydroxybutyryl-CoA is converted to crotonyl-CoA by crotonase, crotonyl-CoA is converted to butyryl-CoA by butyryl-CoA dehydrogenase (bcd). Bcd requires the presence and activity of electron transfer proteins (etfA and etfB) in order to couple the reduction of crotonyl-CoA to the oxidation of NADH. Butyryl-CoA is then converted to butyraldehyde and butyraldehyde is converted to butanol by butyraldehyde dehydrogenase/butanol dehydrogenase. The enzymes may be from C. acetobutylicum.

An example of the second part of the pathway for the conversion of acetyl-CoA to n-butanol using a heterologously expressed pathway with the genes from solventogenic bacteria, for example from Clostridium species, is described in the U.S. patent application Ser. No. 11/949,724, filed Dec. 3, 2007, which is hereby incorporated herein by reference.

In some embodiments, the recombinant microorganism may express one or more heterologous genes encoding for enzymes that confer the capability to produce n-butanol. For example, recombinant microorganisms may express heterologous genes encoding one or more of an anaerobically active pyruvate dehydrogenase (Pdh), Pyruvate formate lyase (Pfl), NADH-dependent formate dehydrogenase (Fdh), acetyl-CoA-acetyltransferase (thiolase), hydroxybutyryl-CoA dehydrogenase, crotonase, butyryl-CoA dehydrogenase, butyraldehyde dehydrogenase, n-butanol dehydrogenase, bifunctional butyraldehyde/n-butanol dehydrogenase. Such heterologous DNA sequences are preferably obtained from a heterologous microorganism (such as Clostridium acetobutylicum or Clostridium beijerinckii), and one or more of these heterologous genes may be introduced into an appropriate host using conventional molecular biology techniques. These heterologous DNA sequences enable the recombinant microorganism to produce n-butanol, at least to produce n-butanol or the metabolic intermediate(s) thereof in an amount greater than that produced by the wild-type counterpart microorganism.

In certain embodiments, the recombinant microorganism herein disclosed expresses a heterologous Thiolase or acetyl-CoA-acetyltransferase, such as one encoded by a thl gene from a Clostridium.

Thiolase (E.C. 2.3.1.19) or acetyl-CoA acetyltransferase, is an enzyme that catalyzes the condensation of an acetyl group onto an acetyl-CoA molecule. The enzyme is, in C. acetobutylicum, encoded by the gene thl (GenBank accession U08465, protein ID AAA82724.1), which was overexpressed, amongst other enzymes, in E. coli under its native promoter for the production of acetone (Bermejo et al., Appl. Environ. Mirobiol. 64:1079-1085, 1998). Homologous enzymes have also been identified, and may be identified by performing a BLAST search against above protein sequence. These homologs can also serve as suitable thiolases in a heterologously expressed n-butanol pathway. Just to name a few, these homologous enzymes include, but are not limited to, those from C. acetobutylicum sp. (e.g., protein ID AAC26026.1), C. pasteurianum (e.g., protein ID ABA18857.1), C. beijerinckii sp. (e.g., protein ID EAP59904.1 or EAP59331.1), Clostridium perfringens sp. (e.g., protein ID ABG86544.1, ABG83108.1), Clostridium difficile sp. (e.g., protein ID CAJ67900.1 or ZP_{—}01231975.1), Thermoanaerobacterium thermosaccharolyticum (e.g., protein ID CAB07500.1), Thermoanaerobacter tengcongensis (e.g., AAM23825.1), Carboxydothermus hydrogenoformans (e.g., protein ID ABB13995.1), Desulfotomaculum reducens MI-1 (e.g., protein ID EAR45123.1), Candida tropicalis (e.g., protein ID BAA02716.1 or BAA02715.1), Saccharomyces cerevisiae (e.g., protein ID AAA62378.1 or CAA30788.1), Bacillus sp., Megasphaera elsdenii, and Butryivibrio fibrisolvens. In addition, the endogenous S. cerevisiae thiolase could also be active in a hetorologously expressed n-butanol pathway (ScERG10).

Homologs sharing at least about 55%, 60%, 65%, 70%, 75% or 80% sequence identity, or at least about 65%, 70%, 80% or 90% sequence homology, as calculated by NCBI's BLAST, are suitable thiolase homologs that can be used in recombinant microorganisms of the present invention. Such homologs include, but are not limited to, Clostridium beijerinckii NCIMB 8052 (ZP_{—}00909576.1 or ZP_{—}00909989.1), Clostridium acetobutylicum ATCC 824 (NP_{—}149242.1), Clostridium tetani E88 (NP_{—}781017.1), Clostridium perfringens str. 13 (NP_{—}563111.1), Clostridium perfringens SM101 (YP_{—}699470.1), Clostridium pasteurianum (ABA18857.1), Thermoanaerobacterium thermosaccharolyticum (CAB04793.1), Clostridium difficile QCD-32g58 (ZP_{—}01231975.1), and Clostridium difficile 630 (CAJ67900.1).

In certain embodiments, recombinant microorganisms of the present invention express a heterologous 3-hydroxybutyryl-CoA dehydrogenase, such as one encoded by an hbd gene from a Clostridium.

The 3-hydroxybutyryl-CoA dehydrogenase (BHBD) is an enzyme that catalyzes the conversion of acetoacetyl-CoA to 3-hydroxybutyryl-CoA. Different variants of this enzyme exist that produce either the (S) or the (R) isomer of 3-hydroxybutyryl-CoA. Homologous enzymes can easily be identified by one skilled in the art by, for example, performing a BLAST search against aforementioned C. acetobutylicum BHBD. All these homologous enzymes could serve as a BHBD in a heterologously expressed n-butanol pathway. These homologous enzymes include, but are not limited to: Clostridium kluyveri, which expresses two distinct forms of this enzyme (Miller et al., J. Bacteriol. 138:99-104, 1979), and Butyrivibrio fibrisolvens, which contains a bhbd gene which is organized within the same locus of the rest of its butyrate pathway (Asanuma et al., Current Microbiology 51:91-94, 2005; Asanuma at al., Current Microbiology 47:203-207, 2003). A gene encoding a short chain acyl-CoA dehydrogenase (SCAD) was cloned from Megasphaera elsdenii and expressed in E. coli. In vitro activity could be determined (Becker et al., Biochemistry 32:10736-10742, 1993). Other homologues were identified in other Clostridium strains such as C. kluyveri (Hillmer et al., FEBS Lett. 21:351-354, 1972; Madan et al., Eur. J. Biochem. 32:51-56, 1973), C. beijerinckii, C. thermosaccharolyticum, C. tetani.

In certain embodiments, wherein a BHBD is expressed it may be beneficial to select an enzyme of the same organism the upstream thiolase or the downstream crotonase originate. This may avoid disrupting potential protein-protein interactions between proteins adjacent in the pathway when enzymes from different organisms are expressed.

In certain embodiments, the recombinant microorganism herein disclosed expresses a heterologous crotonase, such as one encoded by a crt gene from a Clostridium.

The crotonases or Enoyl-CoA hydratases are enzymes that catalyze the reversible hydration of cis and trans enoyl-CoA substrates to the corresponding β-hydroxyacyl CoA derivatives. In C. acetobutylicum, this step of the butanoate metabolism is catalyzed by EC 4.2.1.55, encoded by the crt gene (GenBank protein accession AAA95967, Kanehisa, Novartis Found Symp. 247:91-101, 2002; discussion 01-3, 19-28, 244-52). The crotonase (Crt) from C. acetobutylicum has been purified to homogeneity and characterized (Waterson et al., J. Biol. Chem. 247:5266-5271, 1972). It behaves as a homogenous protein in both native and denatured states. The enzyme appears to function as a tetramer with a subunit molecular weight of 28.2 kDa and 261 residues (Waterson et al. report a molecular mass of 40 kDa and a length of 370 residues). The purified enzyme lost activity when stored in buffer solutions at 4° C. or when frozen (Waterson et al., J. Biol. Chem. 247:5266-5271, 1972). The pH optimum for the enzyme is pH 8.4 (Schomburg et al., Nucleic Acids Res. 32:D431-433, 2004). Unlike the mammalian crotonases that have a broad substrate specificity, the bacterial enzyme hydrates only crotonyl-CoA and hexenoyl-CoA. Values of Vₘₐₓ and Kₘ of 6.5×10⁶ moles per min per mole and 3×10⁻⁵ M were obtained for crotonyl-CoA. The enzyme is inhibited at crotonyl-CoA concentrations of higher than 7×10⁵ M (Waterson et al., J. Biol. Chem. 247:5252-5257, 1972; Waterson et al., J. Biol. Chem. 247:5258-5265, 1972).

The structures of many of the crotonase family of enzymes have been solved (Engel et al., J. Mol. Biol. 275:847-859, 1998). The crt gene is highly expressed in E. coli and exhibits a higher specific activity than seen in C. acetobutylicum (187.5 U/mg over 128.6 U/mg) (Boynton et al., J. Bacteriol. 178:3015-3024, 1996). A number of different homologs of crotonase are encoded in eukaryotes and prokaryotes that functions as part of the butanoate metabolism, fatty acid synthesis, (β-oxidation and other related pathways (Kanehisa, Novartis Found Symp. 247:91-101, 2002; discussion 01-3, 19-28, 244-52; Schomburg et al., Nucleic Acids Res. 32:D431-433, 2003). A number of these enzymes have been well studied. Enoyl-CoA hydratase from bovine liver is extremely well-studied and thoroughly characterized (Waterson et al., J. Biol. Chem. 247:5252-5257, 1972). A ClustalW alignment of 20 closest orthologs of crotonase from bacteria is generated. The homologs vary in sequence identity from 40-85%.

Homologs sharing at least about 45%, 50%, 55%, 60%, 65% or 70% sequence identity, or at least about 55%, 65%, 75% or 85% sequence homology, as calculated by NCBI's BLAST, are suitable Crt homologs that can be used in recombinant microorganisms of the present invention. Such homologs include, but are not limited to, Clostridium tetani E88 (NP_{—}782956.1), Clostridium perfringens SM101 (YP_{—}699562.1), Clostridium perfringens str. 13 (NP_{—}563217.1), Clostridium beijerinckii NCIMB 8052 (ZP_{—}00909698.1 or ZP_{—}00910124.1), Syntrophomonas wolfei subsp. wolfei str. Goettingen (YP_{—}754604.1), Desulfotomaculum reducens MI-1 (ZP_{—}01147473.1 or ZP_{—}01149651.1), Thermoanaerobacterium thermosaccharolyticum (CAB07495.1), and Carboxydothermus hydrogenoformans Z-2901 (YP_{—}360429.1).

Studies in Clostridia demonstrate that the crt gene that codes for crotonase is encoded as part of the larger BCS operon. However, studies on B. fibriosolvens, a butyrate producing bacterium from the rumen, show a slightly different arrangement. While Type I B. fibriosolvens have the thl, crt, hbd, bcd, etfA and etfB genes clustered and arranged as part of an operon, Type II strains have a similar cluster but lack the crt gene (Asanuma et al., Curr. Microbiol. 51:91-94, 2005; Asanuma et al., Curr. Microbiol. 47:203-207, 2003). Since the protein is well-expressed in E. coli and thoroughly characterized, the C. acetobutylicum enzyme is the preferred enzyme for the heterologously expressed n-butanol pathway. Other possible targets are homologous genes from Fusobacterium nucleatum subsp. Vincentii (Q7P3U9-Q7P3U9_FUSNV), Clostridium difficile (P45361-CRT_CLODI), Clostridium pasteurianum (P81357-CRT_CLOPA), and Brucella melitensis (Q8YDG2-Q8YDG2_BRUME).

In certain embodiments, the recombinant microorganism herein disclosed expresses a heterologous butyryl-CoA dehydrogenase and if necessary the corresponding electron transfer proteins, such as encoded by the bcd, etfA, and etfB genes from a Clostridium.

The C. acetobutylicum butyryl-CoA dehydrogenase (Bcd) is an enzyme that catalyzes the reduction of the carbon-carbon double bond in crotonyl-CoA to yield butyryl-CoA. This reduction is coupled to the oxidation of NADH. However, the enzyme requires two electron transfer proteins etfA and etfB (Bennett et al., Fems Microbiology Reviews 17:241-249, 1995).

The Clostridium acetobutylicum ATCC 824 genes encoding the enzymes beta-hydroxybutyryl-coenzyme A (CoA) dehydrogenase, crotonase and butyryl-CoA dehydrogenase are clustered on the BCS operon, which GenBank accession number is U17110.

The butyryl-CoA dehydrogenase (Bcd) protein sequence (Genbank accession #AAA95968.1) is given in SEQ ID NO:3.

Homologs sharing at least about 55%, 60%, 65%, 70%, 75% or 80% sequence identity, or at least about 70%, 80%, 85% or 90% sequence homology, as calculated by NCBI's BLAST, are suitable Bcd homologs that can be used in recombinant microorganisms of the present invention. Such homologs include, but are not limited to, Clostridium tetani E88 (NP_{—}782955.1 or NP_{—}781376.1), Clostridium perfringens str. 13 (NP_{—}563216.1), Clostridium beijerinckii (AF494018_{—}2), Clostridium beijerinckii NCIMB 8052 (ZP_{—}00910125.1 or ZP_{—}00909697.1), and Thermoanaerobacterium thermosaccharolyticum (CAB07496.1), Thermoanaerobacter tengcongensis MB4 (NP_{—}622217.1).

Homologs sharing at least about 45%, 50%, 55%, 60%, 65% or 70% sequence identity, or at least about 60%, 70%, 80% or 90% sequence homology, as calculated by NCBI's BLAST, are suitable Hbd homologs that can be used in the recombinant microorganism herein described. Such homologs include, but are not limited to, Clostridium acetobutylicum ATCC 824 (NP_{—}349314.1), Clostridium tetani E88 (NP_{—}782952.1), Clostridium perfringens SM101 (YP_{—}699558.1), Clostridium perfringens str. 13 (NP_{—}563213.1), Clostridium saccharobutylicum (AAA23208.1), Clostridium beijerinckii NCIMB 8052 (ZP_{—}00910128.1), Clostridium beijerinckii (AF494018_{—}5), Thermoanaerobacter tengcongensis MB4 (NP_{—}622220.1), Thermoanaerobacterium thermosaccharolyticum (CAB04792.1), and Alkaliphilus metalliredigenes QYMF (ZP_{—}00802337.1).

The Kₘ of Bcd for butyryl-CoA is 5. C. acetobutylicum bcd and the genes encoding the respective ETFs have been cloned into an E. coli-C. acetobutylicum shuttle vector. Increased Bcd activity was detected in C. acetobutylicum ATCC 824 transformed with this plasmid (Boynton et al., Journal of Bacteriology 178:3015-3024, 1996). The Kₘ of the C. acetobutylicum P262 Bcd for butyryl-CoA is approximately 6 μM (DiezGonzalez et al., Current Microbiology 34:162-166, 1997). Homologues of Bcd and the related ETFs have been identified in the butyrate-producing anaerobes Megasphaera elsdenii (Williamson et al., Biochemical Journal 218:521-529, 1984), Peptostreptococcus elsdenii (Engel et al., Biochemical Journal 125:879, 1971), Syntrophosphora bryanti (Dong et al., Antonie Van Leeuwenhoek International Journal of General and Molecular Microbiology 67:345-350, 1995), and Treponema phagedemes (George et al., Journal of Bacteriology 152:1049-1059, 1982). The structure of the M. elsdenii Bcd has been solved (Djordjevic et al., Biochemistry 34:2163-2171, 1995). A BLAST search of C. acetobutylicum ATCC 824 Bcd identified a vast amount of homologous sequences from a wide variety of species, some of the homologs are listed herein above. Any of the genes encoding these homologs may be used for the subject invention. It is noted that expression issues, electron transfer issues, or both issues, may arise when heterologously expressing these genes in one microorganism (such as E. coli) but not in another. In addition, one homologous enzyme may have expression and/or electron transfer issues in a given microorganism, but other homologous enzymes may not. The availability of different, largely equivalent genes provides more design choices when engineering the recombinant microorganism.

One promising bcd that has already been cloned and expressed in E. coli is from Megasphaera elsdenii, and in vitro activity of the expressed enzyme could be determined (Becker et al., Biochemistry 32:10736-10742, 1993). O'Neill et al. reported the cloning and heterologous expression in E. coli of the etfA and eftB genes and functional characterization of the encoded proteins from Megasphaera elsdenii (O'Neill et al., J. Biol. Chem. 273:21015-21024, 1998). Activity was measured with the ETF assay that couples NADH oxidation to the reduction of crotonyl-CoA via Bcd. The activity of recombinant ETF in the ETF assay with Bcd is similar to that of the native enzyme as reported by Whitfield and Mayhew. Therefore, utilizing the Megasphaera elsdenii Bcd and its ETF proteins provides a solution to synthesize butyryl-CoA. The Kₘ of the M. elsdenii Bcd was measured as 5 μM when expressed recombinantly, and 14 μM when expressed in the native host (DuPlessis et al., Biochemistry 37:10469-77, 1998). M. elsdenii Bcd appears to be inhibited by acetoacetate at extremely low concentrations (Kᵢ of 0.1 μM) (Vanberkel et al., Eur. J. Biochem. 178:197-207, 1988). A gene cluster containing thl, crt, hbd, bcd, etfA, and etfB was identified in two butyrate producing strains of Butyrivibrio fibrisolvens. The amino acid sequence similarity of these proteins is high, compared to Clostridium acetobutylicum (Asanuma et al., Current Microbiology 51:91-94, 2005; Asanuma et al., Current Microbiology 47:203-207, 2003). In mammalian systems, a similar enzyme, involved in short-chain fatty acid oxidation is found in mitochondria.

In certain embodiments, the recombinant microorganism herein disclosed expresses a heterologous “trans-2-enoyl-CoA reductase” or “TER”.

Trans-2-enoyl-CoA reductase or TER is a protein that is capable of catalyzing the conversion of crotonyl-CoA to butyryl-CoA. In certain embodiments, the recombinant microorganism expresses a TER which catalyzes the same reaction as Bcd/EtfA/EtfB from Clostridia and other bacterial species. Mitochondrial TER from E. gracilis has been described, and many TER proteins and proteins with TER activity derived from a number of species have been identified forming a TER protein family (U.S. Pat. Appl. 2007/0022497 to Cirpus et al.; Hoffmeister et al., J. Biol. Chem., 280:4329-4338, 2005, both of which are incorporated herein by reference in their entirety). A truncated cDNA of the E. gracilis gene has been functionally expressed in E. coli. This cDNA or the genes of homologues from other microorganisms can be expressed together with the n-butanol pathway genes thl, crt, adhE2, and hbd to produce n-butanol in E. coli, S. cerevisiae or other hosts.

TER proteins can also be identified by generally well known bioinformatics methods, such as BLAST. Examples of TER proteins include, but are not limited to, TERs from species such as: Euglena spp. including, but not limited to, E. gracilis, Aeromonas spp. including, but not limited, to A. hydrophila, Psychromonas spp. including, but not limited to, P. ingrahamii, Photobacterium spp. including, but not limited, to P. profundum, Vibrio spp. including, but not limited, to V angustum, V. cholerae, V alginolyticus, V parahaemolyticus, V vulnificus, V fischeri, V splendidus, Shewanella spp. including, but not limited to, S. amazonensis, S. woodyi, S. frigidimarina, S. paeleana, S. baltica, S. denitrificans, Oceanospirillum spp., Xanthomonas spp. including, but not limited to, X oryzae, X campestris, Chromohalobacter spp. including, but not limited, to C. salexigens, Idiomarina spp. including, but not limited, to I. baltica, Pseudoalteromonas spp. including, but not limited to, P. atlantica, Alteromonas spp., Saccharophagus spp. including, but not limited to, S. degradans, S. marine gamma proteobacterium, S. alpha proteobacterium, Pseudomonas spp. including, but not limited to, P. aeruginosa, P. putida, P. fluorescens, Burkholderia spp. including, but not limited to, B. phytofirmans, B. cenocepacia, B. cepacia, B. ambifaria, B. vietnamensis, B. multivorans, B. dolosa, Methylbacillus spp. including, but not limited to, M. flageliatus, Stenotrophomonas spp. including, but not limited to, S. maltophilia, Congregibacter spp. including, but not limited to, C. litoralis, Serratia spp. including, but not limited to, S. proteamaculans, Marinomonas spp., Xytella spp. including, but not limited to, X fastidiosa, Reinekea spp., Colweffia spp. including, but not limited to, C. psychrerythraea, Yersinia spp. including, but not limited to, Y. pestis, Y. pseudotuberculosis, Methylobacillus spp. including, but not limited to, M flagellatus, Cytophaga spp. including, but not limited to, C. hutchinsonii, Flavobacterium spp. including, but not limited to, F. johnsoniae, Microscilla spp. including, but not limited to, M marina, Polaribacter spp. including, but not limited to, P. irgensii, Clostridium spp. including, but not limited to, C. acetobutylicum, C. beijerenckii, C. cellulolyticum, Coxiella spp. including, but not limited to, C. burnetii.

In addition to the foregoing, the terms “trans-2-enoyl-CoA reductase” or “TER” refer to proteins that are capable of catalyzing the conversion of crotonyl-CoA to butyryl-CoA and which share at least about 40%, 45%, 50%, 55%, 60%, 65%, 70%, 75%, 80%, 85%, 90%, 95%, 96%, 97%, 98%, 99% or greater sequence identity, or at least about 50%, 60%, 70%, 80%, 90%, 95%, 96%, 97%, 98%, 99% or greater sequence similarity, as calculated by NCBI BLAST, using default parameters, to either or both of the truncated E. gracilis TER or the full length A. hydrophila TER.

As used herein, “sequence identity” refers to the occurrence of exactly the same nucleotide or amino acid in the same position in aligned sequences. “Sequence similarity” takes approximate matches into account, and is meaningful only when such substitutions are scored according to some measure of “difference” or “sameness” with conservative or highly probably substitutions assigned more favorable scores than non-conservative or unlikely ones.

Another advantage of using TER instead of Bcd/EffA/EffB is that TER is active as a monomer and neither the expression of the protein nor the enzyme itself is sensitive to oxygen.

As used herein, “trans-2-enoyl-CoA reductase (TER) homologue” refers to an enzyme homologous polypeptides from other organisms, e.g., belonging to the phylum Euglena or Aeromonas, which have the same essential characteristics of TER as defined above, but share less than 40% sequence identity and 50% sequence similarity standards as discussed above. Mutations encompass substitutions, additions, deletions, inversions or insertions of one or more amino acid residues. This allows expression of the enzyme during an aerobic growth and expression phase of the n-butanol process, which could potentially allow for a more efficient biofuel production process.

In certain embodiments, the recombinant microorganism herein disclosed expresses a heterologous butyraldehyde dehydrogenase/n-butanol dehydrogenase, such as encoded by the bdhA/bdhB, aad, or adhE2 genes from a Clostridium.

The Butyraldehyde dehydrogenase (BYDH) is an enzyme that catalyzes the NADH-dependent reduction of butyryl-CoA to butyraldehyde. Butyraldehyde is further reduced to n-butanol by an n-butanol dehydrogenase (BDH). This reduction is also accompanied by NADH oxidation. Clostridium acetobutylicum contains genes for several enzymes that have been shown to convert butyryl-CoA to n-butanol.

One of these enzymes is encoded by aad (Nair et al., J. Bacteriol. 176:871-885, 1994). This gene is referred to as adhE in C. acetobutylicum strain DSM 792. The enzyme is part of the sol operon and it encodes for a bifunctional BYDH/BDH (Fischer et al., Journal of Bacteriology 175:6959-6969, 1993; Nair et al., J. Bacteriol. 176:871-885, 1994).

The gene product of aad was functionally expressed in E. coli. However, under aerobic conditions, the resulting activity remained very low, indicating oxygen sensitivity. With a greater than 100-fold higher activity for butyraldehyde compared to acetaldehyde, the primary role of Aad is in the formation of n-butanol rather than of ethanol (Nair et al., Journal of Bacteriology 176:5843-5846, 1994).

Homologs sharing at least about 50%, 55%, 60% or 65% sequence identity, or at least about 70%, 75% or 80% sequence homology, as calculated by NCBI's BLAST, are suitable homologs that can be used in the recombinant microorganisms herein disclosed. Such homologs include (without limitation): Clostridium tetani E88 (NP_{—}781989.1), Clostridium perfringens str. 13 (NP_{—}563447.1), Clostridium perfringens ATCC 13124 (YP_{—}697219.1), Clostridium perfringens SM101 (YP_{—}699787.1), Clostridium beijerinckii NCIMB 8052 (ZP_{—}00910108.1), Clostridium acetobutylicum ATCC 824 (NP_{—}149199.1), Clostridium difficile 630 (CAJ69859.1), Clostridium difficile QCD-32g58 (ZP_{—}01229976.1), and Clostridium thermocellum ATCC 27405 (ZP_{—}00504828.1).

Two additional NADH-dependent n-butanol dehydrogenases (BDH I, BDH II) have been purified, and their genes (bdhA, bdhB) cloned. The GenBank accession for BDH I is AAA23206.1, and the protein sequence is given in SEQ ID NO:10.

The GenBank accession for BDH II is AAA23207.1, and the protein sequence is given in SEQ ID NO:11.

These genes are adjacent on the chromosome, but are transcribed by their own promoters (Walter et al., Gene 134:107-111, 1993). BDH I utilizes NADPH as the cofactor, while BDH II utilizes NADH. However, it is noted that the relative cofactor preference is pH-dependent. BDH I activity was observed in E. coli lysates after expressing bdhA from a plasmid (Petersen et al., Journal of Bacteriology 173:1831-1834, 1991). BDH II was reported to have a 46-fold higher activity with butyraldehyde than with acetaldehyde and is 50-fold less active in the reverse direction. BDH I is only about two-fold more active with butyraldehyde than with acetaldehyde (Welch et al., Archives of Biochemistry and Biophysics 273:309-318, 1989). Thus in one embodiment, BDH II or a homologue of BDH II is used in a heterologously expressed n-butanol pathway. In addition, these enzymes are most active under a relatively low pH of 5.5, which trait might be taken into consideration when choosing a suitable host and/or process conditions.

While the afore-mentioned genes are transcribed under solventogenic conditions, a different gene, adhE2 is transcribed under alcohologenic conditions (Fontaine et al., J. Bacteriol. 184:821-830, 2002, GenBank accession #AF321779). These conditions are present at relatively neutral pH. The enzyme has been overexpressed in anaerobic cultures of E. coli and with high NADH-dependent BYDH and BDH activities. In certain embodiments, this enzyme is the preferred enzyme. The protein sequence of this enzyme (GenBank accession #AAK09379.1) is listed as SEQ ID NO:1.

Homologs sharing at least about 50%, 55%, 60% or 65% sequence identity, or at least about 70%, 75% or 80% sequence homology, as calculated by NCBI's BLAST, are suitable homologs that can be used in the recombinant microorganisms herein disclosed. Such homologs include, but are not limited to, Clostridium perfringens SM101 (YP_{—}699787.1), Clostridium perfringens str. 13 (NP_{—}563447.1), Clostridium perfringens ATCC 13124 (YP_{—}697219.1), Clostridium tetani E88 (NP_{—}781989.1), Clostridium beijerinckii NCIMB 8052 (ZP_{—}00910108.1), Clostridium difficile QCD-32g58 (ZP_{—}01229976.1), Clostridium difficile 630 (CAJ69859.1), Clostridium acetobutylicum ATCC 824 (NP_{—}149325.1), and Clostridium thermocellum ATCC 27405 (ZP_{—}00504828.1).

In certain embodiments, any homologous enzymes that are at least about 70%, 80%, 90%, 95%, 99% identical, or sharing at least about 60%, 70%, 80%, 90%, 95% sequence homology (similar) to any of the above polypeptides may be used in place of these wild-type polypeptides. These enzymes sharing the requisite sequence identity or similarity may be wild-type enzymes from a different organism, or may be artificial, recombinant enzymes.

In certain embodiments, any genes encoding for enzymes with the same activity as any of the above enzymes may be used in place of the genes encoding the above enzymes. These enzymes may be wild-type enzymes from a different organism, or may be artificial, recombinant or engineered enzymes.

Additionally, due to the inherent degeneracy of the genetic code, other nucleic acid sequences which encode substantially the same or a functionally equivalent amino acid sequence can also be used to clone and express the polynucleotides encoding such enzymes. As will be understood by those of skill in the art, it can be advantageous to modify a coding sequence to enhance its expression in a particular host. The codons that are utilized most often in a species are called optimal codons, and those not utilized very often are classified as rare or low-usage codons. Codons can be substituted to reflect the preferred codon usage of the host, a process sometimes called “codon optimization” or “controlling for species codon bias.” Methodology for optimizing a nucleotide sequence for expression in a plant is provided, for example, in U.S. Pat. No. 6,015,891, and the references cited therein]

In certain embodiments, the recombinant microorganism herein disclosed has one or more heterologous DNA sequence(s) from a solventogenic Clostridia, such as Clostridium acetobutylicum or Clostridium beijerinckii. An exemplary Clostridium acetobutylicum is strain ATCC824, and an exemplary Clostridium beijerinckii is strain NCIMB 8052.

Expression of the genes may be accomplished by conventional molecular biology means. For example, the heterologous genes can be under the control of an inducible promoter or a constitutive promoter. The heterologous genes may either be integrated into a chromosome of the host microorganism, or exist as an extra-chromosomal genetic elements that can be stably passed on (“inherited”) to daughter cells. Such extra-chromosomal genetic elements (such as plasmids, BAC, YAC, etc.) may additionally contain selection markers that ensure the presence of such genetic elements in daughter cells.

In certain embodiments, the recombinant microorganism herein disclosed may also produce one or more metabolic intermediate(s) of the n-butanol-producing pathway, such as acetoacetyl-CoA, hydroxybutyryl-CoA, crotonyl-CoA, butyryl-CoA, or butyraldehyde, and/or derivatives thereof, such as butyrate.

In some embodiments, the recombinant microorganisms herein described engineered to activate one or more of the above mentioned heterologous enzymes for the production of n-butanol, produce n-butanol via a heterologous pathway.

As used herein, the term “pathway” refers to a biological process including one or more enzymatically controlled chemical reactions by which a substrate is converted into a product. Accordingly, a pathway for the conversion of a carbon source to n-butanol is a biological process including one or more enzymatically controlled reaction by which the carbon source is converted into n-butanol. A “heterologous pathway” refers to a pathway wherein at least one of the at least one or more chemical reactions is catalyzed by at least one heterologous enzyme. On the other hand, a “native pathway” refers to a pathway wherein the one or more chemical reactions is catalyzed by a native enzyme.

In certain embodiments, the recombinant microorganism herein disclosed are engineered to activate an n-butanol producing heterologous pathway (herein also indicated as n-butanol pathway) that comprises: (1) Conversion of 2 Acetyl-CoA to Acetoacetyl-CoA, (2) Conversion of Acetoacetyl CoA to Hydroxybutyryl-CoA, (3) Conversion of Hydroxybutyryl-CoA to Crotonyl-CoA, (4) Conversion of Crotonyl CoA to Butyryl-CoA, (5) Conversion of Butyraldehyde to n-butanol, (see the exemplary illustration of FIG. 2).

The conversion of 2 Acetyl-CoA to Acetoacetyl-CoA can be performed by expressing a native or heterologous gene encoding for an acetyl-CoA-acetyl transferase (thiolase) or Thl in the recombinant microorganism. Exemplary thiolases suitable in the recombinant microorganism herein disclosed are encoded by thl from Clostridium acetobutylicum, and in particular from strain ATCC824 or a gene encoding a homologous enzyme from C. pasteurianum, C. beijerinckii, in particular from strain NCIMB 8052 or strain BA101, Candida tropicalis, Bacillus spp., Megasphaera elsdenii, or Butyrivibrio fibrisolvens, or an E. coli thiolase selected from fadA or atoB.

The conversion of Acetoacetyl CoA to Hydroxybutyryl-CoA can be performed by expressing a native or heterologous gene encoding for hydroxybutyryl-CoA dehydrogenase Hbd in the recombinant microorganism. Exemplary Hbd suitable in the recombinant microorganism herein disclosed are encoded by hbd from Clostridium acetobutylicum, and in particular from strain ATCC824, or a gene encoding a homologous enzyme from Clostridium kluyveri, Clostridium beijerinckii, and in particular from strain NCIMB 8052 or strain BA101, Clostridium thermosaccharolyticum, Clostridium tetani, Butyrivibrio fibrisolvens, Megasphaera elsdenii, or E. coli (fadB).

The conversion of Hydroxybutyryl-CoA to Crotonyl-CoA can be performed by expressing a native or heterologous gene encoding for a crotonase or Crt in the recombinant microorganism. Exemplary crt suitable in the recombinant microorganism herein disclosed are encoded by crt from Clostridium acetobutylicum, and in particular from strain ATCC824, or a gene encoding a homologous enzyme from B. fibriosolvens, Fusobacterium nucleatum subsp. Vincentii, Clostridium difficile, Clostridium pasteurianum, or Brucella melitensis.

The conversion of Crotonyl CoA to Butyryl-CoA can be performed by expressing a native or heterologous gene encoding for a butyryl-CoA dehydrogenase in the recombinant microorganism. Exemplary butyryl-CoA dehydrogenases suitable in the recombinant microorganism herein disclosed are encoded by bcd/etfA/etfB from Clostridium acetobutylicum, and in particular from strain ATCC824, or a gene encoding a homologous enzyme from Megasphaera elsdenii, Peptostreptococcus elsdenii, Syntrophosphora bryanti, Treponema phagedemes, Butyrivibrio fibrisolvens, or a mammalian mitochondria Bcd homolog.

The conversion of Butyraldehyde to n-butanol can be performed by expressing a native or heterologous gene encoding for a butyraldehyde dehydrogenase or a n-butanol dehydrogenase in the recombinant microorganism. Exemplary butyraldehyde dehydrogenase/n-butanol dehydrogenase suitable in the recombinant microorganism herein disclosed are encoded by bdhA, bdhB, aad, or adhE2 from Clostridium acetobutylicum, and in particular from strain ATCC824, or a gene encoding ADH-1, ADH-2, or ADH-3 from Clostridium beijerinckii, in particular from strain NCIMB 8052 or strain BA101.

In certain embodiments, the enzymes of the metabolic pathway from acetyl-CoA to n-butanol are (i) thiolase (Thl), (ii) hydroxybutyryl-CoA dehydrogenase (Hbd), (iii) crotonase (Crt), (iv) at least one of alcohol dehydrogenase (AdhE2), or n-butanol dehydrogenase (Aad) or butyraldehyde dehydrogenase (Ald) together with a monofunctional n-butanol dehydrogenase (BdhA/BdhB), and (v) trans-2-enoyl-CoA reductase (TER) (FIG. 2). In certain embodiments, the Thl, Hbd, Crt, AdhE2, Ald, BdhA/BdhB and Aad are from Clostridium. In certain embodiments, the Clostridium is a C. acetobutylicum. In certain embodiments, the TER is from Euglena gracilis or from Aeromonas hydrophila.

In certain embodiments, one or more heterologous genes encodes one or more of acetyl-CoA-acetyltransferase (thiolase), hydroxybutyryl-CoA dehydrogenase (hbd), crotonase (crt), and alcohol dehydrogenase (adhE2), butyryl-CoA dehydrogenase (bcd), butyraldehyde dehydrogenase (bdhA/bdhB)/butanol dehydrogenase (aad), and trans-2-enoyl-CoA reductase (TER).

For example, the acetyl-CoA-acetyltransferase (thiolase) may be thl from Clostridium acetobutylicum, or a homologous enzyme from C. pasteurianum, Clostridium beijerinckii, Candida tropicalis, Bacillus sp., Megasphaera elsdenii, or Butryivibrio fibrisolvens, or an E. coli thiolase selected from fadA or atoB.

The hydroxybutyryl-CoA dehydrogenase may be hbd from C. acetobutylicum, or a homologous enzyme from Clostridium kluyveri, Clostridium beijerinckii, Clostridium thermosaccharolyticum, Clostridium tetani, Butyrivibrio fibrisolvens, Megasphaera elsdenii, or Escherichia coli (fadB).

The crotonase may be crt from Clostridium acetobutylicum, or a homologous enzyme from B. fibriosolvens, Fusobacterium nucleatum subsp. Vincentii, Clostridium difficile, Clostridium pasteurianum, or Brucella melitensis.

The butyryl-CoA dehydrogenase may be bcd/etfA/etfB from Clostridium acetobutylicum, or a homologous enzyme from Megasphaera elsdenii, Peptostreptococcus elsdenii, Syntrophosphora bryanti, Treponema phagedemes, Butyrivibrio fibrisolvens, or a eukaryotic mitochondrial bcd homolog.

The butyraldehyde dehydrogenase/butanol dehydrogenase may be bdhA, bdhB, aad, or adhE2 from Clostridium acetobutylicum, or ADH-1, ADH-2, or ADH-3 from Clostridium beijerinckii.

The enzyme trans-2-enoyl-CoA reductase (TER), may be from a Euglena gracilis or an Aeromonas hydrophila.

The one or more heterologous DNA sequence(s) may be from a solventogenic Clostridium selected from Clostridium acetobutylicum or Clostridium beijerinckii, or from Clostridium difficile, Clostridium pasteurianum, Clostridium kluyveri, Clostridium thermosaccharolyticum, Clostridium tetani, Candida tropicalis, Bacillus sp., Brucella melitensis, Megasphaera elsdenii, Butryivibrio fibrisolvens, Fusobacterium nucleatum subsp. Vincentii, Peptostreptococcus elsdenii, Syntrophosphora bryanti, Treponema phagedemes, or E. coli.

In certain embodiments, the Clostridium acetobutylicum is strain ATCC824, and the Clostridium beijerinckii is strain NCIMB 8052 or strain BA101. In certain embodiments, homologs sharing at least 40%, 45%, 50%, 55%, 60%, 65%, 70%, 75%, 80%, 85%, 90% sequence identity, or at least about 50%, 60%, 70%, 80%, 90% sequence identity (as calculated by NCBI BLAST, using default parameters) are suitable for the subject invention.

Part (1): Engineering the Conversion of Pyruvate to acetyl-CoA

As described above, the conversion of pyruvate to acetyl-CoA may occur in an engineered cell by two general routes: (A) the “PDH bypass” route as defined above or (B) the direct conversion of pyruvate to acetyl-CoA in the cytosol by PDH or by PFL.

(A) Acetyl-CoA Generation Via the “PDH Bypass” Route

Relating to the route (A) in generating acetyl CoA from pyruvate, the cytosolic acetyl-CoA generation pathway is catalyzed by three enzymes as shown in FIG. 3, Steps 1, 2 and 3. A more efficient pathway for generation of acetyl-CoA is achieved by increasing the activity of those enzymes that are rate-limiting. For example, in Saccharomyces cerevisiae, if ALD activity is limiting in a pathway, overexpression of ALD6 will thereby increase the overall flux through the pathway. Increased acetyl-CoA formation in the cytosol is achieved via one of the following mechanisms or a combination thereof:

In one embodiment, increased acetyl-CoA may be generated by the overexpression of a pyruvate decarboxylase gene (for example, S. cerevisiae PDC1, PDC5 and/or PDC6; Step 1).

In another embodiment, increased acetyl-CoA may be generated by the overexpression of an acetaldehyde dehydrogenase gene (for example, S. cerevisiae ALD6; Step 2).

In yet another embodiment, increased acetyl-CoA may be produced by the overexpression of an acetyl-CoA synthase gene (for example, S. cerevisiae ACS1 or ACS2 or both; Step 3).

In a different embodiment, simultaneous overexpression of both ALD and ACS (S. cerevisiae ALD6; Step 2) may generate increased acetyl-CoA (Steps 2 and 3).

In another embodiment, simultaneous overexpression of PDC, ALD, and ACS genes may generate increased production of acetyl-CoA (Steps 1-3).

To further increase production of acetyl-CoA, the major cytosolic ethanol production pathway in yeast can be reduced or eliminated. In Crabtree positive, S. cerevisiae, this is achieved by the deletion of ADH1 which is the predominant source of cytosolic ADH activity. Cells deleted for ADH1 are unable to grow anaerobically (Drewke et al., (1990). J. Bacteriology 172(7):3909), and thus may be preferably deleted to minimize conversion of acetaldehyde to ethanol. Eliminating this pathway selectively drives acetaldehyde towards acetate and subsequently to acetyl-CoA production (FIG. 3, Step 5). Therefore, overexpression of the genes described above may be carried out in a cell having reduced or eliminated ADH activity.

Similarly, cytosolic ADH activity may be reduced or eliminated in a Crabtree negative yeast such as Kluyveromyces lactis by the deletion of ADHI or ADHII to increase the flux from pyruvate to acetyl-CoA via the “PDH bypass” route. Therefore, in this organism, similar to that proposed to S. cerevisiae above, the flux via the “PDH bypass” route could be increased by the over-expression of KIALD6, KIACS1 or KIACS2 alone or in combination.

(B) Direct Generation of Acetyl-CoA from Pyruvate

Relating to the route (B) of generating acetyl CoA from pyruvate, acetyl-CoA production may be increased by the overexpression of the genes forming a complete PDH complex. For example, the overexpressed genes may be from E. coli (aceE, aceF, and lpdA), Zymomonas mobilis (pdhAα, pdhAβ, pdhB, and lpd), S. aureus (pdhA, pdhB, pdhC, and lpd), Bacillus subtilis, Corynebacterium glutamicum, or Pseudomonas aeruginosa (Step 4).

Pyruvate dehydrogenase enzyme complex catalyzes the conversion of pyruvate to acetyl-CoA. In S. cerevisiae, this complex is localized in the mitochondrial inner membrane space. Consequently, another method to obtain higher levels of acetyl-CoA in the cytoplasm of S. cerevisiae is to engineer a cell to overexpress a eukaryotic or prokaryotic pyruvate dehydrogenase complex which can function in the cytoplasm (Step 4). In certain embodiments, the recombinant microorganism herein disclosed includes an active pyruvate dehydrogenase (Pdh) under anaerobic or microaerobic conditions. The pyruvate dehydrogenase or NADH-dependent formate dehydrogenase may be heterologous to the recombinant microorganism, in that the coding sequence encoding these enzymes is heterologous, or the transcriptional regulatory region is heterologous (including artificial), or the encoded polypeptides comprise sequence changes that renders the enzyme resistant to feedback inhibition by certain metabolic intermediates or substrates.

Until recently, it was widely accepted that Pdh does not function under anaerobic conditions, but several recent reports have demonstrated that this is not the case (de Graef, M. et al, 1999, Journal of Bacteriology, 181, 2351-57; Vernuri, G. N. et al, 2002, Applied and Environmental Microbiology, 68, 1715-27). Moreover, other microorganisms such as Enterococcus faecalis exhibit high in vivo activity of the Pdh complex, even under anaerobic conditions, provided that growth conditions were such that the steady-state NADH/NAD⁺ ratio was sufficiently low (Snoep, J. L. et al, 1991, Fems Microbiology Letters, 81, 63-66). Instead of oxygen regulating the expression and function of Pdh, it has been shown that Pdh is regulated by NADH/NAD⁺ ratio (de Graef, M. et al, 1999, Journal of Bacteriology, 181, 2351-57. If the n-butanol pathway expressed in a host cell consumes NADH fast enough to maintain a low NADH/NAD⁺ level inside the cell, an endogenous or heterologously expressed Pdh may remain active and provide NADH sufficient to balance the pathway.

These Pdh enzymes can balance the n-butanol pathway in a recombinant microorganism herein disclosed.

Expression of a Pdh that is functional under anaerobic conditions is expected to increase the moles of NADH obtained per mole of glucose. Kim et al. describe a Pdh that makes available in E. coli up to four moles of NADH per mole of glucose consumed (Kim, Y. et al. (2007). Appl. Environm. Microbiol., 73, 1766-1771). Yeast cells can also be engineered to express PDH complexes from diverse bacterial sources. For example, Pdh from Enterococcus faecalis is similar to the Pdh from E. coli but is inactivated at much lower NADH/NAD⁺ levels. Additionally, some organisms such as Bacillus subtilis and almost all strains of lactic acid bacteria use a Pdh in anaerobic metabolism. Expression of an n-butanol production pathway in a microorganism expressing an Pdh that is anaerobically active is expected to result in n-butanol yields of greater than 1.4% if the n-butanol production pathway can compete with endogenous fermentative pathways.

Alternatively, acetyl-CoA may be produced in the cytosol by overexpressing two bacterial enzymes, a pyruvate formate lyase (e.g., E. coli pflB) and a formate dehydrogenase (e.g., Candida boidinii fdh1). Using this pathway, pyruvate is converted to acetyl-CoA and formate. Formate dehydrogenase then catalyzes the NADH-dependent conversion of formate to carbon dioxide. The net result of these reactions is the same as if pyruvate was converted to acetyl-CoA by pyruvate dehydrogenase complex:

Pyruvate+NAD⁺→acetyl-CoA+NADH+CO₂.

NADH-dependent formate dehydrogenase (Fdh; EC 1.2.1.2) catalyzes the oxidation of formate to CO₂ and the simultaneous reduction of NAD⁺ to NADH. Fdh can be used in accordance with the present invention to increase the intracellular availability of NADH within the host microorganism and may be used to balance the n-butanol producing pathway with respect to NADH. In particular, a biologically active NADH-dependent Fdh can be activated and in particular overexpressed in the host microorganism. In the presence of this newly introduced formate dehydrogenase pathway, one mole of NADH will is formed when one mole of formate is converted to carbon dioxide. In certain embodiments, in the native microorganism a formate dehydrogenase converts formate to CO₂ and H₂ with no cofactor involvement.

Furthermore any of the genes encoding the foregoing enzymes (or any others mentioned herein (or any of the regulatory elements that control or modulate expression thereof) may be subject to directed evolution using methods known to those of skill in the art. Such action allows those of skill in the art to optimize the enzymes for expression and activity in yeast.

In addition, pyruvate decarboxylase, acetyl-CoA synthetase, and acetaldehyde dehydrogenase genes from other fungal and bacterial species can be expressed for the modulation of this pathway. A variety of organisms could serve as sources for these enzymes, including, but not limited to, Saccharomyces sp., including S. cerevisiae mutants and S. uvarum, Kluyveromyces, including K. thermotolerans, K. lactis, and K. mandanus, Pichia, Hansenula, including H. polymorpha, Candidia, Trichosporon, Yamadazyma, including Y. stipitis, Torulaspora pretoriensis, Schizosaccharomyce pombe, Cryptococcus sp., Aspergillus sp., Neurospora sp. or Ustilago sp. Examples of useful pyruvate decarboxylase are those from Saccharomyces bayanus (1PYD), Candida glabrata, K. lactis (KIPDC1), or Aspergillus nidulans (PdcA), and acetyl-CoA sythetase from Candida albicans, Neurospora crassa, A. nidulans, or K. lactis (ACS1), and acetaldehyde dehydrogenase from Aspergillus niger (ALDDH), C. albicans, Cryptococcus neoformans (alddh). Sources of prokaryotic enzymes that are useful include, but are not limited to, E. coli, Z. mobilis, Bacillus sp., Clostridium sp., Pseudomonas sp., Lactococcus sp., Enterobacter sp. and Salmonella sp. Further enhancement of this pathway can be obtained through engineering of these enzymes for enhanced activity by site-directed mutagenesis and other evolution methods (which include techniques known to those of skill in the art).

Prokaryotes such as, but not limited to, E. coli, Z. mobilis, Staphylococcus aureus, Bacillus sp., Clostridium sp., Corynebacterium sp., Pseudomonas sp., Lactococcus sp., Enterobacter sp., and Salmonella sp., can serve as sources for this enzyme complex. For example, pyruvate dehydrogenase complexes from E. coli (aceE, aceF, and lpdA), Z. mobilis (pdhAalpha, pdhAbeta, pdhB, and lpd), S. aureus (pdhA, pdhB, pdhC, and pdhC), Bacillus subtilis, Corynebacterium glutamicum, and Pseudomonas aeruginosa, can be used for this purpose.

Methods to grow and handle yeast are well known in the art. Methods to overexpress, express at various lower levels, repress expression of, and delete genes in yeast cells are well known in the art and any such method is contemplated for use to construct the yeast strains of the present.

Any method can be used to introduce an exogenous nucleic acid molecule into yeast and many such methods are well known to those skilled in the art. For example, transformation, electroporation, conjugation, and fusion of protoplasts are common methods for introducing nucleic acid into yeast cells. See, e.g., Ito et al., J. Bacterol. 153:163-168 (1983); Durrens et al., Curr. Genet. 18:7-12 (1990); and Becker and Guarente, Methods in Enzymology 194:182-187 (1991).

In an embodiment, the integration of a gene of interest into a DNA fragment or target gene occurs according to the principle of homologous recombination. According to this embodiment, an integration cassette containing a module comprising at least one yeast marker gene, with or without the gene to be integrated (internal module), is flanked on either side by DNA fragments homologous to those of the ends of the targeted integration site (recombinogenic sequences). After transforming the yeast with the cassette by appropriate methods, a homologous recombination between the recombinogenic sequences may result in the internal module replacing the chromosomal region in between the two sites of the genome corresponding to the recombinogenic sequences of the integration cassette.

In an embodiment, for gene deletion, the integration cassette may include an appropriate yeast selection marker flanked by the recombinogenic sequences. In an embodiment, for integration of a heterologous gene into the yeast chromosome, the integration cassette includes the heterologous gene under the control of an appropriate promoter and terminator together with the selectable marker flanked by recombinogenic sequences. In an embodiment, the heterologous gene comprises an appropriate native gene desired to increase the copy number of a native gene(s). The selectable marker gene can be any marker gene used in yeast, including, but not limited to, URA3 gene from S. cerevisiae or a homologous gene; or hygromycin resistance gene for auxotrophy complementation or antibiotic resistance-based selection of the transformed cells, respectively. The recombinogenic sequences can be chosen at will, depending on the desired integration site suitable for the desired application.

Additionally, in an embodiment, certain introduced marker genes are removed from the genome using techniques well known to those skilled in the art. For example, URA3 marker loss can be obtained by plating URA3 containing cells in FOA (5-fluoro-orotic acid) containing medium and selecting for FOA resistant colonies (Boeke, J. et al, 1984, Mol. Gen. Genet, 197, 345-47).

The exogenous nucleic acid molecule contained within a yeast cell of the disclosure can be maintained within that cell in any form. For example, exogenous nucleic acid molecules can be integrated into the genome of the cell or maintained in an episomal state that can stably be passed on (“inherited”) to daughter cells. Such extra-chromosomal genetic elements (such as plasmids, etc.) can additionally contain selection markers that ensure the presence of such genetic elements in daughter cells. Moreover, the yeast cells can be stably or transiently transformed. In addition, the yeast cells described herein can contain a single copy, or multiple copies, of a particular exogenous nucleic acid molecule as described above.

Methods for expressing a polypeptide from an exogenous nucleic acid molecule are well known to those skilled in the art. Such methods include, without limitation, constructing a nucleic acid such that a regulatory element promotes the expression of a nucleic acid sequence that encodes the desired polypeptide. Typically, regulatory elements are DNA sequences that regulate the expression of other DNA sequences at the level of transcription. Thus, regulatory elements include, without limitation, promoters, enhancers, and the like. For example, the exogenous genes can be under the control of an inducible promoter or a constitutive promoter. Moreover, methods for expressing a polypeptide from an exogenous nucleic acid molecule in yeast are well known to those skilled in the art. For example, nucleic acid constructs that are capable of expressing exogenous polypeptides within Kluyveromyces (see, e.g., U.S. Pat. Nos. 4,859,596 and 4,943,529, each of which is incorporated by reference herein in its entirety) and Saccharomyces (see, e.g., Gelissen et al., Gene 190(1):87-97 (1997)) are well known. In another embodiment, heterologous control elements can be used to activate or repress expression of endogenous genes. Additionally, when expression is to be repressed or eliminated, the gene for the relevant enzyme, protein or RNA can be eliminated by known deletion techniques.

As described herein, yeast within the scope of the disclosure can be identified by selection techniques specific to the particular enzyme being expressed, over-expressed or repressed. Methods of identifying the strains with the desired phenotype are well known to those skilled in the art. Such methods include, without limitation, PCR and nucleic acid hybridization techniques such as Northern and Southern analysis, altered growth capabilities on a particular substrate or in the presence of a particular substrate, a chemical compound, a selection agent and the like. In some cases, immunohistochemistry and biochemical techniques can be used to determine if a cell contains a particular nucleic acid by detecting the expression of the encoded polypeptide. For example, an antibody having specificity for an encoded enzyme can be used to determine whether or not a particular yeast cell contains that encoded enzyme. Further, biochemical techniques can be used to determine if a cell contains a particular nucleic acid molecule encoding an enzymatic polypeptide by detecting a product produced as a result of the expression of the enzymatic polypeptide. For example, transforming a cell with a vector encoding acetyl-CoA synthetase and detecting increased cytosolic acetyl-CoA concentrations indicates the vector is both present and that the gene product is active. Methods for detecting specific enzymatic activities or the presence of particular products are well known to those skilled in the art. For example, the presence of acetyl-CoA can be determined as described by Dalluge et al., Anal. Bioanal. Chem. 374(5):835-840 (2002).

Yeast cells of the present invention have reduced enzymatic activity such as reduced alcohol dehydrogenase activity. The term “reduced” as used herein with respect to a cell and a particular enzymatic activity refers to a lower level of enzymatic activity than that measured in a comparable yeast cell of the same species. Thus yeast cells lacking alcohol dehydrogenase activity is considered to have reduced alcohol dehydrogenase activity since most, if not all, comparable yeast strains have at least some alcohol dehydrogenase activity. Such reduced enzymatic activities can be the result of lower enzyme concentration, lower specific activity of an enzyme, or a combination thereof. Many different methods can be used to make yeast having reduced enzymatic activity. For example, a yeast cell can be engineered to have a disrupted enzyme-encoding locus using common mutagenesis or knock-out technology. See, e.g., Methods in Yeast Genetics (1997 edition), Adams, Gottschling, Kaiser, and Stems, Cold Spring Harbor Press (1998).

Alternatively, antisense technology can be used to reduce enzymatic activity. For example, yeast can be engineered to contain a cDNA that encodes an antisense molecule that prevents an enzyme from being made. The term “antisense molecule” as used herein encompasses any nucleic acid molecule that contains sequences that correspond to the coding strand of an endogenous polypeptide. An antisense molecule also can have flanking sequences (e.g., regulatory sequences). Thus antisense molecules can be ribozymes or antisense oligonucleotides. A ribozyme can have any general structure including, without limitation, hairpin, hammerhead, or axhead structures, provided the molecule cleaves RNA.

Yeast having a reduced enzymatic activity can be identified using any method. For example, yeast having reduced alcohol dehydrogenase activity can be easily identified using common methods, for example, by measuring ethanol formation via gas chromatography.

In one embodiment, n-butanol can be produced from one of the metabolically-engineered strains of the present disclosure using a two-step process. Because high levels of butanol (e.g., 1.5% in the media and this generally varies by yeast and strain) can be toxic to the cells, one strategy to obtain large quantities of n-butanol is to grow a strain capable of producing n-butanol under conditions in which no butanol, or only an insignificant, non-toxic amount of butanol, is produced. This step allows accumulation of a large quantity of viable cells, i.e., a significant amount of biomass, which can then be shifted to growth conditions under which n-butanol is produced. Such a strategy allows a large amount of n-butanol to be produced before toxicity problems become significant and slow cell growth. For example, cells can be grown under aerobic conditions (in which n-butanol production is suppressed or absent) then shifted to anaerobic or microaerobic conditions to produce n-butanol (e.g., by activation of the appropriate metabolic pathways that have been engineered into the strain in accordance with the present invention). Alternatively, expression of the relevant enzymes can be under inducible control, e.g., thermal sensitive promoters or other thermal sensitive step (such as the thermostability of the enzyme itself), so the first step takes place with the relevant pathway(s) or enzymes turned off (i.e., inactive), induction takes place (e.g., temperature shift), and n-butanol is produced. Methods for making genes subject to inducible control are well known. Thermostable enzymes are known or can be selected by methods know in the art. As in other processes of the disclosure, once n-butanol is produced, it can be recovered in accordance with an embodiment.

Processes for recovering n-butanol from microorganisms, including yeast are disclosed in U.S. Provisional application Ser. No. 11/949,724, filed Dec. 3, 2007, which is hereby incorporated herein by reference.

It will be appreciated by those skilled in the art that various omissions, additions and modifications may be made to the invention described above without departing from the scope of the invention, and all such modifications and changes are intended to fall within the scope of the invention, as defined by the appended claims. All references, patents, patent applications, or other documents cited are hereby incorporated herein by reference.

### EXAMPLES

Table 1 lists a set of genes that are described in Examples 1-38. The relevant primers (forward and reverse) that may be used to amplify each gene, as well as the sequence of each primer, are given. Genes are listed according to the nomenclature conventions appropriate for each species; certain genes as listed are preceded by two letters, representing the first letter of the genus and species of origin for a given gene. For certain gene names, the suffix “-co” is attached to indicate that a codon-optimized, synthetic gene was constructed using preferred codon usage for either the bacterium E. coli, or the yeast S. cerevisiae, as indicated in the text.

TABLE 1

| | | | | |
|---|---|---|---|---|
| | Gene | | | |
| | SEQ | | SEQ | |
| | ID | primer | ID | |
| Gene | NO: | name | NO: | primer sequence |

| | | | | |
|---|---|---|---|---|
| Cb-hbd | 155 | Gevo-311 | 42 | GAGGTTGTCGACATGAAAAAGATTTTTGTACTTGGAG |
| | | Gevo-175 | 43 | AATTGGATCCTTATTTAGAATAATCATAGAATCCT |
| Cb-crt | 156 | Gevo-312 | 44 | GTTCTTGTCGACATGGAATTAAAAAATGTTATTCTTG |
| | | Gevo-171 | 45 | AATTGGATCCTTATTTATTTTGAAAATTCTTTTCTGC |
| Cb-bcd | 157 | Gevo-313 | 46 | CAAGAGGTCGACATGAATTTCCAATTAACTAGAGAAC |
| | | Gevo-314 | 47 | GCGTCCGGATCCCTATCTTAAAATGCTTCCTGCG |
| Cb-etfA | 158 | Gevo-315 | 48 | CGGAAAGTCGACATGAATATAGCAGATTACAAAGGC |
| | | Gevo-173 | 49 | AATTGGATCCTTATTCAGCGCTCTTTATTTCTTTA |
| Cb-etfB | 159 | Gevo-316 | 50 | CAAAATGTCGACATGAATATAGTAGTTTGTGTAAAAC |
| | | Gevo-317 | 51 | TAATTTGGATCCTTAGATGTAGTGTTTTTCTTTTAAT |
| Cb-adA | 160 | Gevo-319 | 52 | GAACCAGTCGACATGGCACGTTTTACTTTACCAAG |
| | | Gevo-177 | 53 | AATTGGATCCTTACAAATTAACTTTAGTTCCATAG |
| Cb-aldh | 161 | Gevo-318 | 54 | TCCATAGTCGACATGAATAAAGACACACTAATACCT |
| | | Gevo-249 | 55 | AATTGGATCCTTAGCCGGCAAGTACACATCTTCTTTGTCT |
| Ca-thl | 162 | Gevo-308 | 56 | GATCGAGTCGACATGAAAGAAGTTGTAATAGCTAG |
| | | Gevo-309 | 57 | GTTATAGGATCCCTAGCACTTTTCTAGCAATATTG |
| Ca-hbd | 163 | Gevo-281 | 58 | GTGGATGTCGACATGAAAA.AGGTATGTGTTATAGGTG |
| | | Gevo-161 | 59 | AATTGGATCCTTATTTTGAATAATCGTAGAAACCT |
| Ca-crt | 164 | Gevo-282 | 60 | TCCTACGTCGACATGGAACTAAACAATGTCATCCT |
| | | Gevo-283 | 61 | TAACTTGGATCCCTATCTATTTTTGAAGCCTTCAAT |
| Ca-bcd | 165 | Gevo-284 | 62 | CAAGAGGTCGACATGGATTTTAATTTAACAAGAGAAC |
| | | Gevo-285 | 63 | CAATAAGGATCCTTATCT,AAAAATTTTTCCTGAAATAAC |
| Ca-etfA | 166 | Gevo-286 | 64 | CGGGAAGTCGACATGAATAAAGCAGATTACAAGGGC |
| | | Gevo-287 | 65 | GTTCAAGGATCCTT,AATTATTAGCAGCTTTAACTTG |
| Ca-etfB | 167 | Gevo-288 | 66 | CAAAATTGTCGACATGAATATAGTTGTTTGTTTAAAAC |
| | | Gevo-289 | 67 | GTTTTAGGATCCTTAAATATAGTGTTCTTCTTTTAATTTTG |
| Ca-adhE2 | 168 | Gevo-292 | 68 | CAAGAAGTCGACATGAAAGTTACAAATCAAAAAGAAC |
| | | Gevo-293 | 69 | TCCTATGCGGCCGCTTAAAATGATTTTATATAGATATCCT |
| Ca-aad | 169 | Gevo-290 | 70 | AGGAAAGTCGACATGAAAGTCACAACAGTAAAGGA |
| | | Gevo-291 | 71 | ATTTAAGCGGCCGCTTAAGGTTGTTTTTTAAAACAATTTA |
| Ca-bdhA | 170 | Gevo-294 | 72 | CATAACGTCGACATGCTAAGTTTTGATTATTCAATAC |
| | | Gevo-247 | 73 | AATTGGATCCTTAATAAGATTTTTTAAATATCTCAA |
| Ca-bdhB | 171 | Gevo-295 | 74 | CATAACGTCGACATGGTTGATTTCGAATATTCAATAC |
| | | Gevo-159 | 75 | AATTGGATCCTTACACAGATTTTTTGAATATTTGTA |
| Ca-thl- | 1 | Gevo-310 | 76 | GATCGAGAATTCATGAAAGAAGTTGTAATAGCTAG |
| co | | | | |
| | | Gevo-309 | 77 | GTTATAGGATCCCTAGCACTTTTCTAGCAATATTG |
| Ca-hbd- | 2 | Gevo-296 | 78 | CGGATAGTCGACATGAAAAAGGTATGTGTTATAGGC |
| co | | | | |
| | | Gevo-297 | 79 | TCCCAAGGATCCTTATTTTGAATAATCGTAGAAACCCT |
| Ca-crt- | 3 | Gevo-282 | 80 | TCCTACGTCGACATGGAACTAAACAATGTCATCCT |
| co | | | | |
| | | Gevo-283 | 81 | TAACTTGGATCCCTATCTATTTTTGAAGCCTTCAAT |
| Ca-bcd- | 4 | Gevo-284 | 82 | CAAGAGGTCGACATGGATTTTAATTTAACAAGAGAAC |
| co | | | | |
| | | Gevo-298 | 83 | GTAAAGGGATCCTTAACTAAAAATTTTTCCTGAAATG |
| Ca-eftA- | 5 | Gevo-286 | 84 | CGGGAAGTCGACATGAATAAAGCAGATTACAAGGGC |
| co | | | | |
| | | Gevo-299 | 85 | GTTCAAGGATCCTTAATTATTAGCAGCTTTAACCTG |
| Ca-eftB- | 6 | Gevo-288 | 86 | CAAAATTGTCGACATGAATATAGTTGTTTGTTTAAAAC |
| co | | | | |
| | | Gevo-300 | 87 | GACTTTGGATCCTTAAATATAGTGTTCTTCTTTCAG |
| Ca- | 7 | Gevo-292 | 88 | CAAGAAGTCGACATGAAAGTTACAAATCAAAAAGAAC |
| adhE2- | | | | |
| co | | | | |
| | | Gevo-301 | 89 | ATTTTCGGATCCTTAAAATGATTTTATATAGATATCTTTTA |
| Me-bcd- | 8 | Gevo-302 | 90 | CTTATAGTCGACATGGATTTTAACTTAACAGATATTC |
| co | | | | |
| | | Gevo-303 | 91 | CCGCCAGGATCCTTAACGTAACAGAGCACCGCCGGT |
| Me-eftA- | 9 | Gevo-304 | 92 | CGGAAAGTCGACATGGATTTAGCAGAATACAAAGGC |
| co | | | | |
| | | Gevo-305 | 93 | CTTTGTGGATCCTTATGCAATGCCTTTCTGTTTC |
| Me-eftB- | 10 | Gevo-306 | 94 | CAAACTGAATTCATGGAAATATTGGTATGTGTCAAAC |
| co | | | | |
| | | Gevo-307 | 95 | ACCAACGGATCCTTAAATGATTTTCTGGGCAACCA |
| ERG10 | 154 | Gevo-273 | 96 | GTTACAGTCGACATGTCTCAGAACGTTTACATTG |
| | | Gevo-274 | 97 | GATAACGGATCCTCATATCTTTTCAATGACAATAG |
| IpdA | 20 | Gevo-610 | 119 | ttttGTCGACACTAGTatgagtactgaaatcaaaactcaggtcgtg |
| | | Gevo-611 | 120 | ttttCTCGAGttacttcttcttcgctttcgggttcgg |
| aceE | 21 | Gevo-606 | 116 | ttttGTCGACACTAGTatgtcagaacgtttcccaaatgacgtgg |
| | | Gevo-607 | 117 | ttttCTCGAGttacgccagacgcgggttaactttatctg |
| aceF | 22 | Gevo-653 | 136 | ttttGTCGACACTAGTatggctatcgaaatcaaagtaccggacatcggg |
| | | Gevo-609 | 118 | ttttCTCGAGttacatcaccagacggcgaatgtcagacag |
| PDA1 | 23 | Gevo-660 | 143 | ttttCTCGACactagtATGgcaactttaaaaacaactgataagaagg |
| | | Gevo-66 1 | 144 | ttttagatctTTAATCCCTAGAGGCAAAACCTTGC |
| PDB1 | 24 | Gevo-662 | 145 | ttttCTCGACactagtATGgcggaagaattggaccgtgatgatg |
| | | Gevo-663 | 146 | tttGGATCCTTATTCAATTGACAAGACTTCTTTGACAG |
| PDX1 | 25 | Gevo-664 | 147 | TtttCTCGACactagtATGttacttgctgtaaagacattttcaatgcc |
| | | Gevo-665 | 148 | ttttggatccTCAAAATGATTCTAACTCCCTTACGTAATC |
| LAT1 | 26 | Gevo-656 | 139 | ttttCTCGAGgctagcATGGCATCGTACCCAGAGCACACCATTATTGG |
| | | Gevo-657 | 140 | ttttGGATCCTCACAATAGCATTTCCAAAGGATTTTCAAT |
| LPD1 | 27 | Gevo-658 | 141 | ttttCTCGACactagtATGGTCATCATCGGTGGTGGCCCTGCTGG |
| | | Gevo-659 | 142 | ttttGGATCCTCAACAATGAATAGCTTTATCATAGG |
| PDC1 | 28 | Gevo-639 | 129 | ttttctcgagactagtATGTCTGAAATTACTTTGGG |
| | | Gevo-640 | 130 | ttttggatccTTATTGCTTAGCGTTGGTAGCAGCAG |
| CUPI | 178 | Gevo-637 | 127 | ttttGAGCTCgccgatcccattaccgacatttggg |
| prom | | | | |
| | | Gevo-638 | 128 | aaaGTCGACaccgatatacctgtatgtgtcaccaccaatgtatctataagtatc |
| | | | | catGCTAGCCCTAGGtttatgtgatgattgattgattgattg |
| pflA | 36 | PflA_forw | 98 | cattgaattcatgtcagttattggtcgcattcac |
| | | PflA_Rev | 99 | catt tcgacttagaacattaccttatgaccgtactg |
| pflB | 37 | PflB_forw | 100 | cattgaattcatgtccgagcttaatgaaaagttagcc |
| | | PflB_Rev | 101 | cattgtcgacttacatagattgagtgaaggtacgag |
| Cb- | 138 | fdh1_forw | 102 | cattgaattcatgaagatcgttttagtcttatatggtgc |
| FDH1 | | | | |
| | | fdh1_rev | 103 | cattgtcgacttatttcttatcgtgtttaccgtaagc |
| KIALD6 | 39 | KIALD6_right | 104 | gttaggatccttaatccaacttgatcctgacggccttg |
| | | KIALD6_Left5 | 105 | ccaagtcgacatgtcctctacaattgctgagaaattgaacctc |
| KIACS1 | 40 | KIACS1_Right3 | 106 | gttagcggccgcttataatttcacggaatcgatcaagtgc |
| | | KIACS1_Left5 | 107 | ccaagctagcatgtctcctgctgttgataccgcttcc |
| KIACS2 | 41 | KIACS2_right3 | 108 | ggttggatccttatttcttctgctgactgaaaaattgattttctactgc |
| | | KIACS2_Left5 | 109 | ccaagaattcatgtcgtcggataaattgcataagg |
| ACS1 | 30 | Gevo-479 | 112 | catgccgtcgacatgtcgccctctgccgtcc |
| | | Gevo-480 | 113 | gattaagcggccgcttacaacttgaccgaatcaattag |
| ACS2 | 31 | Gevo-483 | 114 | gatgaagtcgacatgacaatcaaggaacataaagtag |
| | | Gevo-484 | 115 | gttaaaggatccttatttctttttttgagagaaaaattg |
| ALD6 | 29 | Gevo-643 | 133 | ccaagtcgacatgactaagctacactttgacac |
| | | Gevo-644 | 134 | gtcggtaagagtgttgctgtggactcg |
| Ca-ter | 179 | Gevo-345 | 183 | atgtttgtcgacatgatagtaaaagcaaagtttgta |
| | | Gevo-346 | 184 | cttaatgcggccgcttaaggttctaattttcttaataattc |
| Ah-ter | 180 | Gevo-343 | 185 | Gcttgagtcgacatgatcattaaaccgaaagttcg |
| | | Gevo-344 | 186 | atttaaggatcctcacagttcgacaacatcaaattta |
| Eg-ter | 181 | Gevo-347 | 187 | catcacgtcgacatggccatgttcaccactac |
| | | Gevo-348 | 188 | ctcgcgggatccttactgctgagctgcgctc |
| Sc-ccr | 182 | Gevo-341 | 189 | gtcttagtcgacatgaccgtgaaagacattctg |
| | | Gevo-342 | 190 | attggcggatcctcacacattacggaaacggtta |

Table 2 lists a set of plasmid constructs and their relevant features, as described in the Examples. Included in the table are the relevant plasmid name (pGV); the prototrophic marker present, useful for selection and maintenance of the plasmid in an appropriate auxotrophic strain; a promoter sequence (from the given S. cerevisiae gene region); the gene under control of the aforementioned promoter; additional promoter+gene combinations, if present.

TABLE 2
Summary of relevant features of plasmids in Examples.

| | Prototrophic | | | | |
|---|---|---|---|---|---|
| Name | marker | Promoter 1 | GENE 1 | Promoter 2 | GENE 2 |
| pGV1099 | HIS3 | TEF1 | (AU1 tag) | | |
| pGV1100 | TRP1 | TEF1 | (HA tag) | | |
| pGV1101 | LEU2 | TEF1 | (AU1 tag) | | |
| pGV1102 | URA3 | TEF1 | (HA tag) | | |
| pGV1103 | HIS3 | TDH3 | (myc tag) | | |
| pGV1104 | TRP1 | TDH3 | (myc tag) | | |
| pGV1105 | LEU2 | TDH3 | (myc tag) | | |
| pGV1106 | URA3 | TDH3 | (myc tag) | | |
| pGV1208 | TRP1 | TEF1 | Ca-hbd-co | | |
| pGV1209 | LEU2 | TEF1 | Ca-crt-co | | |
| pGV1213 | URA3 | TEF1 | Ca-adhE2-co | | |
| pGV1214 | HIS3 | TDH3 | Me-bcd-co | | |
| pGV1217 | TRP1 | TEF1 | Ca-hbd-co | TDH3 | Ca-eftA-co |
| pGV1218 | LEU2 | TEF1 | Ca-crt-co | TDH3 | Ca-eftB-co |
| pGV1219 | HIS3 | TEF1 | ScERG10 | TDH3 | Me-bcd-co |
| pGV1220 | HIS3 | TEF1 | Ca-thl-co | TDH3 | Ca-bcd-co |
| pGV1221 | TRP1 | TEF1 | Ca-hbd-co | TDH3 | Me-eftA-co |
| pGV1222 | LEU2 | TEF1 | Ca-crt-co | TDH3 | Me-eftB-co |
| pGV1223 | HIS3 | TEF1 | ScERG10 | TDH3 | Ca-bcd-co |
| pGV1224 | HIS3 | TEF1 | Ca-thl-co | TDH3 | Me-bcd-co |
| pGV1225 | HIS3 | TEF1 | Ca-thl-co | TDH3 | Ca-ter |
| pGV1226 | HIS3 | TEF1 | Ca-thl-co | TDH3 | Ah-ter |
| pGV1227 | HIS3 | TEF1 | Ca-thl-co | TDH3 | Eg-ter |
| pGV1228 | HIS3 | TEF1 | Ca-thl-co | TDH3 | Sc-ccr |
| pGV1262 | LEU2 | TEF1 | ScACS1 | | |
| pGV1263 | URA3 | TEF1 | ScACS2 | | |
| pGV1319 | URA3 | TDH3 | Ca-AdhE2_co | TEF1 | ACS1 |
| pGV1320 | URA3 | TDH3 | Ca-AdhE2_co | TEF1 | ACS2 |
| pGV1321 | LEU2 | TDH3 | ALD6 | | |
| pGV1326 | LEU2 | TEF1 | ALD6 | | |
| pGV1334 | HIS3 | TDH3 | lpdA | | |
| pGV1339 | LEU2 | TEF1 | Ca_Crt_co | TDH3 | ALD6 |
| pGV1379 | HIS3 | TDH3 | aceE | | |
| pGV1380 | HIS3 | TDH3 | aceF | | |
| pGV1381 | HIS3 | TDH3 | LAT1 | | |
| pGV1383 | HIS3 | TDH3 | PDA1 | | |
| pGV1384 | HIS3 | TDH3 | PDB1 | | |
| pGV1385 | HIS3 | TDH3 | PDX1 | | |
| pGV1389 | URA3 | CUP1 | n/a | | |
| pGV1389 | URA3 | TDH3 | PDC1 | | |
| pGV1399 | LEU2 | TEF1 | Ca-hbd-co | TDH3 | ALD6 |
| pGV1414 | URA3 | MET3 | n/a | | |
| pGV1428 | HIS3 | TDH3 | n/a | | |
| pGV1429 | TRP1 | TDH3 | n/a | | |
| pGV1430 | LEU2 | TDH3 | n/a | | |
| pGV1483 | URA3 | MEt3 | n/a | | |
| pGV1603 | TRP1 | TDH3 | aceE | | |
| pGV1604 | LEU2 | TDH3 | aceF | | |
| pGV1605 | URA3 | TEF1 | adhE2 | TDH3 | PDC1 |
| 1102Fdh1 | URA3 | TEF1 | Cb-FDH1 | | |
| 1103PflA | HIS3 | TDH3 | pflA | | |
| 1104PflB | TRP1 | TDH3 | pflB | | |
| 1208_PflA | TRP1 | TEF1 | Ca_hbd_co | TDH3 | pflA |
| 1208KI | HIS3 | TEF1 | Ca_hbd_co | | |
| 1208KIALD6 | HIS3 | TEF1 | Ca_hbd_co | TDH3 | KIALD6 |
| 1208KIPflA | HIS3 | TEF1 | Ca_hbd_co | TDH3 | pflA |
| 1208KIPflA | TRP1 | TEF1 | Ca_Crt_co | TDH3 | pflB |
| 1208-lpdA | TRP1 | TEF1 | thl | TDH3 | -lpdA |
| 1209_PflB | LEU2 | TEF1 | Ca_Crt_co | TDH3 | pflB |
| 1209-aceE | LEU2 | TEF1 | crt | TDH3 | aceE |
| 1209KI | TRP1 | TEF1 | Ca_Crt_co | | |
| 1209kIACS1 | LEU2 | TEF1 | Ca_Crt_co | TDH3 | KIACS1 |
| 1209kIACS2 | LEU2 | TEF1 | Ca_Crt_co | TDH3 | KIACS2 |
| 1213_Fdh1 | URA3 | TDH3 | Ca_AdhE2_co | TEF1 | Cb-FDH1 |
| 1213-aceF | URA3 | TEF1 | adhE2 | TDH3 | aceF |
| 1213KI | URA3 | TDH3 | Ca_AdhE2_co | | |
| 1213KIPflA | LEU2 | TEF1 | Ca_thl_co | TDH3 | Cb-FDH1 |
| 1227KI | LEU2 | TEF1 | Ca_thl_co | TDH3 | Eg-TER-co |
| 1388-PDC1 | URA3 | CUP1 | PDC1 | | |
| 1428_PflA | HIS3 | TDH3 | pflA | | |
| 1428ALD6 | HIS3 | TDH3 | KIALD6 | | |
| 1428-lpdA | HIS3 | TDH3 | -lpdA | | |
| 1429_PflB | TRP1 | TDH3 | pflB | | |
| 1429-aceE | TRP1 | TDH3 | aceE | | |
| 1429ACS1 | TRP1 | TDH3 | KIACS1 | | |
| 1430_Fdh1 | LEU2 | TDH3 | Cb-FDH1 | | |
| 1430-aceF | LEU2 | TDH3 | aceF | | |
| 1431ACS2 | URA3 | TDH3 | KIACS2 | | |
| pGV1103- | HIS3 | TDH3 | LPD1 | | |
| lpd1 | | | | | |

Table 3 describes butanol produced in a yeast, S. cerevisiae (strain W303a), carrying various plasmids, and thereby expressing a set of introduced genes, which are as listed.

TABLE 3
Butanol production by Saccharomyces cerevisiae transformants.

| | Plasmid | | Butanol Amount |
|---|---|---|---|
| Isolate Name | Combination | Introduced Genes | 72 h p.i (μM) |
| Gevo 1094; | pGV1208; | Ca-hbd-co; Ca-Crt-co; | 129; 145 |
| Gevo1095 | pGV1209; | Ca-thl-co + Ca-ter; Ca- | |
| | pGV1225; | adhE2-co | |
| | pGV1213 | | |
| Gevo 1096; | pGV1208; | Ca-hbd; Ca-Crt; Ca-thl- | 207; 216 |
| Gevo1097 | pGV1209; | co + Ah-ter; Ca-adhE2- | |
| | pGV1226; | co | |
| | pGV1213 | | |
| Gevo 1098; | pGV1208; | Ca-hbd; Ca-Crt; Ca-thl- | 251; 313 |
| Gevo1099 | pGV1209; | co + Eg-ter; Ca-adhE2- | |
| | pGV1227; | co | |
| | pGV1213 | | |
| Gevo 1100, | pGV1208; | Ca-hbd; Ca-Crt; Ca-thl- | 109; 109 |
| Gevo1101 | pGV1209; | co + Sc-ter; Ca-adhE2- | |
| | pGV1228; | co | |
| | pGV1213 | | |
| Gevo 1102, | pGV1217; | Ca-hbd-co + Ca-etfa- | 317; 332 |
| Gevo1103 | pGV1218; | co; Ca-Crt-co + Ca-etfb- | |
| | pGV1220; | co; Ca-thl-co + Ca-bcd- | |
| | pGV1213 | co; Ca-adhE2-co | |
| Gevo 1104, | pGV1217; | Ca-hbd-co + Ca-etfa- | 172; 269 |
| Gevo1105 | pGV1218; | co; Ca-Crt-co + Ca-etfb- | |
| | pGV1223; | co; ERG10 + Ca-bcd- | |
| | pGV1213 | co; Ca-adhE2-co | |
| Gevo 1106, | pGV1221; | Ca-hbd-co + Ca-etfa- | 125; 115 |
| Gevo1107 | pGV1222; | co; Ca-Crt-co + Ca-etfb- | |
| | pGV1224; | co; Ca-thl-co + Me-bcd- | |
| | pGV1213 | co; Ca-adhE2-co | |
| Gevo 1108, | pGV1221; | Ca-hbd-co + Ca-etfa- | 101; 124 |
| Gevo1109 | pGV1222; | co; Ca-Crt-co + Ca-etfb- | |
| | pGV1219; | co; ERG10 + Me-bcd- | |
| | pGV1213 | co; Ca-adhE2-co | |
| Gevo 1110, | pGV1099; | N/A | 0; 12 |
| Gevo1111 | pGV1100; | | |
| | pGV1101; | | |
| | pGV1106 | | |

All gene cloning and combination procedures were initially carried out in E. coli using established methods (Miller, J. H., 1992, Sambrook, J. et. al, 2001).

A set of vectors useful for expression in a yeast, S. cerevisiae, has been described previously (Mumberg, D., et al. (1995) Gene 156:119-122; Sikorski & Heiter (1989) Genetics 122:19-27). In particular, these publications describe a set of selectable markers (HIS3, LEU2, TRP1, URA3) and S. cerevisiae replication origins that are also used in many of the vectors listed in Table 2.

### Example 1

### Plasmid Construction for Expression of Butanol Pathway Genes in the Yeast, S. cerevisiae

The S. cerevisiae thiolase gene, ERG10, was cloned by PCR from genomic DNA from the S. cerevisiae strain W303a, using primers which introduced a SalI site immediately upstream of the start codon and a BamHI site immediately after the stop codon. This PCR product was digested with SalI and BamHI and cloned into the same sites of pUC19 (Yanisch-Perron, C., Vieira, J., 1985, Gene, 33, 103-19) to generate pGV1120.

The plasmids pGV1031, pGV1037, pGV1094, and pGV1095 were used as templates for PCR amplification of the C. acetobutylicum genes (Ca-) Ca-thl, Ca-hbd, Ca-crt, and Ca-bdhB, respectively. pGV1090 was used as template for PCR amplification of Ca-bcd, Ca-etfA, and Ca-etfB. Genomic DNA of Clostridium ATCC 824 was used to amplify Ca-bdhA. Amplified fragments were digested with SalI and BamHI and cloned into the same sites of pUC19. This scheme generated plasmids, pGV1121, pGV1122, pGV1123, pGV1124, pGV1125, pGV1126, pGV1127, pGV1128, which contain the genes, Ca-thl, Ca-hbd, Ca-crt, Ca-bcd, Ca-etfA, Ca-etfB, Ca-bdhA, and Ca-bdhB, respectively.

The Clostridium beijerinckii (Cb-) genes, Cb-hbd, Cb-crt, Cb-bcd, Cb-effA, Cb-etfB, Cb-aldh, and Cb-adhA were amplified by PCR using primers designed to introduce a SalI site just upstream of the start and a BamHI site just downstream of the stop codon. The plasmids pGV1050, pGV1049, pGV1096 and pGV1091 were used as templates for PCR amplification of Cb-hbd, Cb-crt, Cb-aldh, and Cb-adhA, respectively. Genomic DNA of Clostridium beijerinckii ATCC 51743 was used as template for Cb-bcd, Cb-etfA, and Cb-etfB. The PCR amplified fragments were digested with SalI and BamHI and cloned into the same sites of pUC19. This procedure generated plasmids pGV1129, pGV1130, pGV1131, pGV1132, pGV1133, pGV1134, and pGV1135, which contain the genes, Cb-hbd, Cb-crt, Cb-bcd, Cb-etfA, Cb-etfB, Cb-aldh, and Cb-adhA, respectively.

The C. acetobutylicum and Meghasphaera elsdenii (Me-) genes that were codon optimized (-co) for expression in E. coli were also cloned. These genes include Ca-thl-co, Ca-hbd-co, Ca-crt-co, Ca-bcd-co, Ca-etfA-co, Ca-etfB-co, Ca-adhE2-co, Me-bcd-co, Me-etfA-co, and Me-etfB-co. These genes, except for Ca-thl-co and Me-etfB-co were amplified using primers designed to introduce a SalI site just upstream of the start codon and a BamHI site just downstream of the stop codon. In the case of Ca-thl-co and Me-etfB-co, primers were designed to introduce an EcoRI site just upstream of the start codon and a BamHI site just downstream of the stop codon. The resulting PCR products were digested using the appropriate restriction enzymes (SalI and BamHI or EcoRI and BamHI) and cloned into the same sites of pUC19 to generate plasmids pGV1197, pGV1198, pGV1199, pGV1200, pGV1201, pGV1202, pGV1203, pGV1205, pGV1206, which contain the genes, Ca-thl-co, Ca-hbd-co, Ca-crt-co, Ca-bcd-co, Ca-etfA-co, Ca-etfB-co, Ca-adhE2-co, Me-etfA-co, and Me-etfB-co, respectively. Me-bcd-co gene was directly cloned into pGV1103 as a SalI-BamHI fragment to generate pGV1214.

The above genes were cloned into high copy yeast expression vectors, pGV1099, pGV1100, pGV1101, pGV1102, pGV1103, pGV1104, pGV1105 and pGV1106. The properties of the vectors used for gene cloning and resulting plasmid constructs are described in Table 2.

The thiolase genes, ERG10 and Ca-thl were released from pGV1120 and pGV1121 using SalI and BamHI and cloned into pGV1099 (carrying a HIS3 marker) to generate pGV1138 and pGV1139, respectively. The codon-optimized thiolase gene, Ca-thl-co was removed from pGV1197 and cloned into pGV1099 using EcoRI and BamHI to generate pGV1207. Thus, these genes are cloned in-frame with two copies of the AU1 tag (SEQ ID NO:172) and expressed using the S. cerevisiae TEF1 promoter region (SEQ ID NO:175). The hydroxybutyryl-CoA-dehydrogenase genes, Ca-hbd (from pGV1122), Cb-hbd (from pGV1129), and Ca-hbd-co (from pGV1198) were cloned into pGV1100 (carries LEU2 marker) using SalI and BamHI to generate pGV1140, pGV1141, and pGV1208, respectively. This results in these genes being cloned in-frame with an HA tag (SEQ ID NO:173) and expressed using the TEF1 promoter. The crotonase genes, Ca-crt (from pGV1123), Cb-crt (from pGV1130), Ca-crt-co (from pGV1199) were cloned into pGV1101 (carries TRP1 marker) using Sail and BamHI to generate pGV1142, pGV1143, and pGV1209, respectively. Thus, these genes are cloned in-frame with two copies of the AU1 tag and expressed using the TEF1 promoter.

The butyryl-CoA dehydrogenase and the respective electron transfer genes etfA and etfB were cloned behind a myc tag (SEQ ID NO:174) expressed using the TDH3 promoter region from S. cerevisiae (SEQ ID NO:176). The Ca-bcd (from pGV1124), Cb-bcd (from pGV1131), Ca-bcd-co (from pGV1200) and Me-bcd-co genes were cloned into pGV1103 (carries HIS3 marker) to generate pGV1144, pGV1145, pGV1210, and pGV1214. The Ca-etfA (from pGV1125), Ca-etfB (from pGV1126), Cb-etfA (from pGV1132), Cb-etfB (from pGV1133), Ca-etfB-co (from pGV1202), and Me-etfA-co (from pGV1205) genes were cloned into pGV1104 (carries LEU2 marker) to generate pGV1146, pGV1147, pGV1148, pGV1149, pGV1212, and pGV1215, respectively. The Ca-etfA-co (from pGV1201) and Me-etfB-co (from pGV1206) were cloned into pGV1104 (carries TRP1 marker) to generate pGV1211 and pGV1216, respectively.

The gene for an aldehyde dehydrogenase, Cb-aldh (from pGV1134), was cloned into pGV1102 (carries URA3 marker) to generate pGV1150. The Cb-aldh gene is placed in frame with the HA tag (SEQ ID NO:173) expressed using the TEF1 promoter. The bi-functional aldehyde/alcohol dehydrogenases, Ca-aad, Ca-adhE2, and Ca-adhE2-co, and the specific alcohol dehydrogenases, Ca-bdhA, Ca-bdhB, and Cb-adhA were cloned behind a myc-tag expressed under the control of the TDH3 promoter. Ca-aad and Ca-adhE2 were amplified by PCR using primers designed to introduce a SalI site just upstream of the start codon and a NotI site just downstream of the stop codon. The plasmid, pGV1089, was used as a template for Ca-aad, and the C. acetobutylicum genomic DNA was used as a template for Ca-adhE2. These PCR products were cloned into pGV1106 (carries URA3 marker) using SalI and NotI to generate pGV1136 (Ca-aad) and pGV1137 (Ca-adhE2). The codon optimized Ca-adhE2-co (from pGV1203) was cloned into pGV1106 using SalI and BamHI to generate pGV1213. The alcohol dehydrogenases, Ca-bdhA (from pGV1127), Ca-bdhB (from pGV1128), and Cb-adhA (from pGV1135), were cloned into pGV1106 using SalI and BamHI to generate pGV1151, pGV1152, and pGV1153, respectively.

Therefore, the above described yeast expression genes for butyryl-coA dehydrogenase, electron transfer protein A, electron transfer protein B, and the specific alcohol dehydrogenase were combined with the TEF1 promoter driven thiolase, hydroxybutyryl-CoA dehydrogenase, crotonase, or the aldehyde dehydrogenase, in pair-wise fashion as summarized in Table 2 above.

For this purpose, the EcoICRI to XhoI fragments from pGV1144 (TDH3 promoter and Ca-bcd) and from pGV1145 (TDH3 promoter and Cb-bcd) were cloned into the NotI (filled in with Klenow) to XhoI sites of pGV1138 to generate pGV1167 (ERG10+Ca-bcd) and pGV1168 (ERG10+Cb-bcd), respectively. These same EcoICRI to XhoI fragments were also similarly cloned into pGV1139 to generate pGV1169 (Ca-thl+Ca-bcd) and pGV1170 (Ca-thl+Cb-bcd), respectively. Using the same strategy, the EcoICRI to XhoI fragments from pGV1146 (TDH3 promoter and Ca-etfA), pGV1148 (TDH3 promoter and Ca-etfB), pGV1147 (TDH3 promoter and Cb-etfA), and pGV1149 (TDH3 promoter and Cb-etfB) were cloned into the NotI (filled in with Klenow) to XhoI sites of pGV1140, pGV1141, pGV1142, pGV1143 to generate pGV1171 (Ca-hbd+Ca-etfA), pGV1172 (Ca-crt+Ca-etfB), pGV1173 (Cb-hbd+Cb-etfA), and pGV1174 (Cb-crt+Cb-etfB), respectively. The aldehyde dehyrogenase and the alcohol dehydrogenases were combined similarly by cloning the EcoICRI to XhoI fragments from pGV1151 (TDH3 promoter and Ca-bdhA), pGV1152 (TDH3 promoter and Ca-bdhB) and pGV1153 (TDH3 promoter and Cb-adhA) into the (filled in with Klenow) to XhoI sites of pGV1150 to generate pGV1175 (Cb-aldh+Ca-bdhA), pGV1176 (Cb-aldh+Ca-bdhB), and pGV1177 (Cb-aldh+Cb-adhA), respectively.

In the case of the codon-optimized genes, the EcoICRI to XhoI fragments from pGV1210 (TDH3 promoter and Ca-bcd-co), pGV1211 (TDH3 promoter and Ca-etfA-co), pGV1212 (TDH3 promoter and Ca-etfB-co) were cloned into the BamHI (filled in with Klenow) to XhoI sites of pGV1207, pGV1208, and pGV1209, respectively to generate pGV1220 (Ca-thl-co+Ca-bcd-co), pGV1217 (Ca-hbd-co+Ca-etfA-co), and pGV1218 (Ca-crt-co+Ca-etfB-co). The EcoICRI to XhoI fragments from pGV1214 (TDH3 promoter and Me-bcd-co), pGV1215 (TDH3 promoter and Me-etfA-co), pGV1216 (TDH3 promoter and Me-etfB-co) were also cloned into the same set of vectors, respectively, to generate pGV1224 (Ca-thl-co+Me-bcd-co), pGV1221 (Ca-hbd-co+Me-etfA-co), and pGV1222 (Ca-crt-co+Me-etfB-co). Furthermore, the EcoICRI to XhoI fragments from pGV1210 (TDH3 promoter and Ca-bcd-co) and from pGV1214 (TDH3 promoter and Me-bcd-co) were cloned into the BamHI (filled in with Klenow) to XhoI sites of pGV1138 to generate pGV1223 (ERG10+Ca-bcd-co) and pGV1219 (ERG10+Me-bcd-co).

In addition to the above pathway, constructs were generated that utilize alternatives to the bcd/etfA/etfB complex, namely trans-enoyl reductase and crotonyl-CoA reductase. Trans-enoyl reductase genes from C. aetobutylicum (Ca-ter), Aeromonas hydrophila (Ah-ter), and Euglena gracilis (Eg-ter) and the crotonyl-coA reductase from Streptomyces collinus (Sc-ccr) were cloned. Ca-ter was PCR amplified from C. acetobutylicum genomic DNA using primers designed to introduce a SalI site immediately upstream of the start codon and a NotI site just downstream of the stop codon. Ah-ter, Eg-ter, and Sc-ccr were PCR amplified from pGV1114, pGV1115, and pGV1166, respectively, using primer designed to introduce a SalI site immediately upstream of the start codon and a BamHI site just downstream of the stop codon. The sequences for these three genes have been codon optimized for expression in E. coli. Also, the Eg-ter sequence encodes for a protein that is missing the N-terminal region which may be involved in mitochondrial localization. The respective PCR products were cloned into pGV1103 using appropriate restriction enzymes to generate pGV1155 (Ca-ter), pGV1156 (Ah-ter), pGV1157 (Eg-ter) and pGV1158 (Sc-ccr).

For use in expressing the butanol pathway in yeast, these alternatives to the bcd/etfA/etfB complex were each combined with a thiolase gene on one plasmid. The Ca-ter, Ah-ter, Eg-ter and Sc-ccr genes were combined with the Ca-thl-co gene by cloning the EcoICRI to XhoI fragment from pGV1155, pGV1156, pGV1157 and pGV1158 into the BamHI (filled in with Klenow) to XhoI sites of pGV1207 to generate pGV1225 (Ca-thl-co+Ca-ter), pGV1226 (Ca-thl-co+Ah-ter), pGV1227 (Ca-thl-co+Eg-ter) and pGV1228 (Ca-thl-co+Sc-ccr), respectively.

### Example 2

### Yeast Extract/Western Blot Analysis

For analysis of protein expression, crude yeast protein extracts were made by a rapid TCA precipitation protocol. One OD600 equivalent of cells was collected and treated with 200 μL of 1.85N NaOH/7.4% 2-mercaptoethanol on ice for 10 mins. 200 μL of 50% TCA was added and the samples incubated on ice for an additional 10 mins. The precipitated proteins were collected by centrifugation at 25,000 rcf for 2 mins and washed with 1 mL of ice cold acetone. The proteins were again collected by centrifugation at 25,000 rcf for 2 mins. The pellet was then resuspended in SDS Sample Buffer and boiled (99° C.) for 10 mins. The samples were centrifuged at maximum in a microcentrifuge for 30 sec to remove insoluble matter.

Samples were separated by a SDS-PAGE and transferred to nitrocellulose. Western analysis was done using the TMB Western Blot Kit (KPL). HA.11, myc (9E10), and AU1 antibodies were obtained from Covance. Westerns were performed as described by manufacturer, except that when the myc antibody was used, detector block solution was used at 0.3×-0.5× supplemented with 1% detector block powder. Expression of all genes described in Example 1, was verified utilizing this method.

### Example 3

### Yeast Transformations

Saccharomyces cerevisiae (W303a) transformations were done using lithium acetate method (Gietz, R. D. a. R. A. W., 2002, Methods in Enzymology, 350, 87-96). Briefly, 1 mL of an overnight yeast culture was diluted into 50 mL of fresh YPD medium and incubated in a 30° C. shaker for 5-6 hours. The cells were collected, washed with 50 mL sterile water, and washed with 25 mL sterile water. The cells were resuspended using 1 mL 100 mM lithium acetate and transferred to a microcentrifuge tube. The cells were pelleted by centrifuging for 10 s. The supernatant was discarded and the cells were resuspended in 4× volume of 100 mM lithium acetate. 15 μL of the cells were added to the DNA mix (72 μL 50% PEG, 10 μL 1M lithium acetate, 3 uL 10 mg/ml denatured salmon sperm DNA, 2 μL each of the desired plasmid DNA and sterile water to a total volume of 100 μL). The samples were incubated at 30° C. for 30 min and heat shocked at 42° C. for 22 min. The cells were then collected by centrifuging for 10 s, resuspended in 100 μL SOS medium (Sambrook, J., Fritsch, E. F., Maniatis, T., 1989), and plated onto appropriate SC selection plates (Kaiser C., M., S, and Mitchel, A, 1994)—without uracil, tryptophan, leucine or histidine.

### Example 4

### Production of n-butanol

Transformants (Table 1 above) expressing different combinations of enzymes related to the proposed butanol production pathway were assessed for n-butanol production. Pre-cultures of the isolates were prepared by inoculating a few colonies from SC agar plates into 3 ml of SC medium (Kaiser C., M., S. and Mitchel, A, 1994) which was shaken under aerobic conditions for 16 hours at 30° C. at 250 rpm. The resulting cells were pelleted at 4000×g for 5 minutes and resuspended in 500 μl of SC medium. Cell growth was assessed by absorbance at 600 nm with suitable dilutions. For each isolate tested, cells yielding 150D were injected (200 μl) into anaerobic balch tubes containing 5 ml of SC anaerobic medium, previously saturated with N₂ gas to remove dissolved oxygen. The tubes were incubated at 30° C. with 250 rpm shaking to prevent cell settling.

The tubes were sampled 10, 26, 44 and 70 hours post-inoculation by removing 500 μl of culture with a sterile syringe. Afterwards, 250 μl of 40% glucose solution was injected into each tube to maintain adequate carbon in the culture medium. At each time point, the recovered samples were centrifuged to pellet the cells and the supernatant was immediately frozen until all the samples were collected.

N-butanol production by the transformants was determined by gas chromotography (GC) analysis. All frozen samples were thawed at room temperature and 400 μl of each sample with 80 μl of 10 mM Pentanol added as an internal control was filtered through a 0.2 μm filter. 200 μl of the resulting filtrate was placed in GC vials and subjected to GC analysis.

Samples were run on a Series II Plus gas chromatograph with a flame ionization detector (FID), fitted with a HP-7673 autosampler system. Analytes were identified based on the retention times of authentic standards and quantified using 5-point calibration curves. All samples were injected at a volume of 1 Direct analysis of the n-butanol product was performed on a DB-FFAP capillary column (30 m length, 0.32 mm ID, 0.25 μm film thickness) connected to the FID detector. The temperature program for separating the alcohol products was 225° C. injector, 225° C. detector, 50° C. oven for 0 minutes, then 8° C./minute gradient to 80° C., 13° C./minute gradient to 170° C., 50° C./minute gradient to 220° C., then 220° C. for 3 minutes.

For evaluation of butanol production, two independent transformants of each plasmid combination were tested. The results are summarized in Table 3 above. The two Gevo numbers under “Isolate Name” refer to the two independent transformants assessed for each plasmid combination.

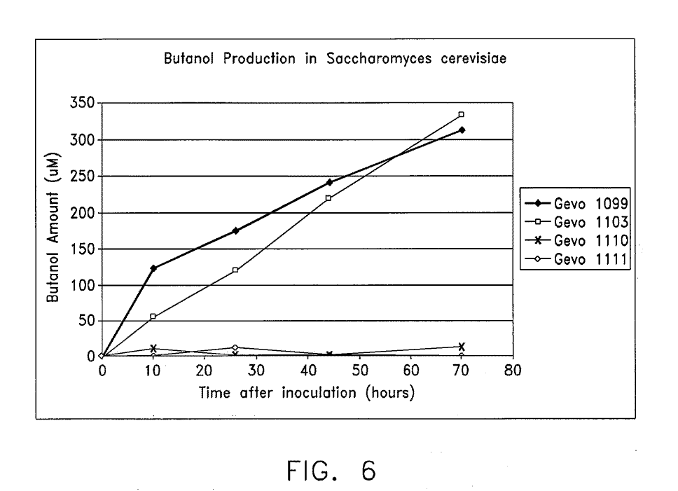

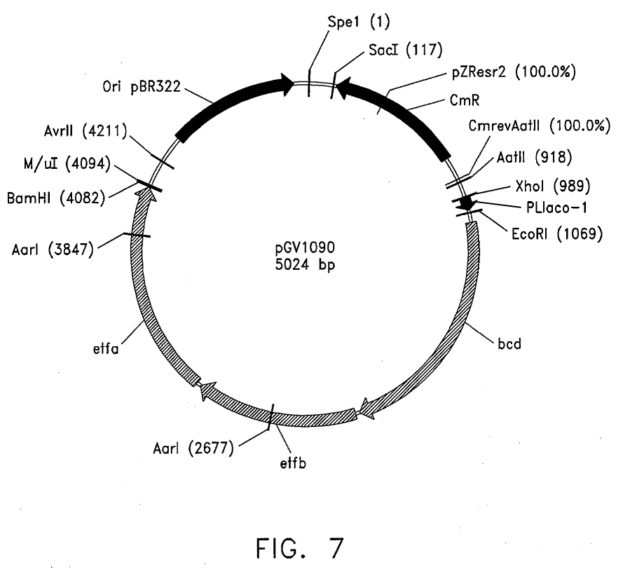

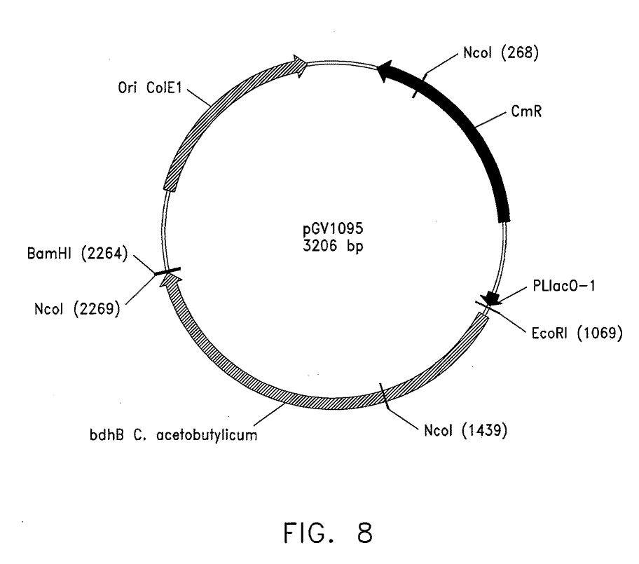

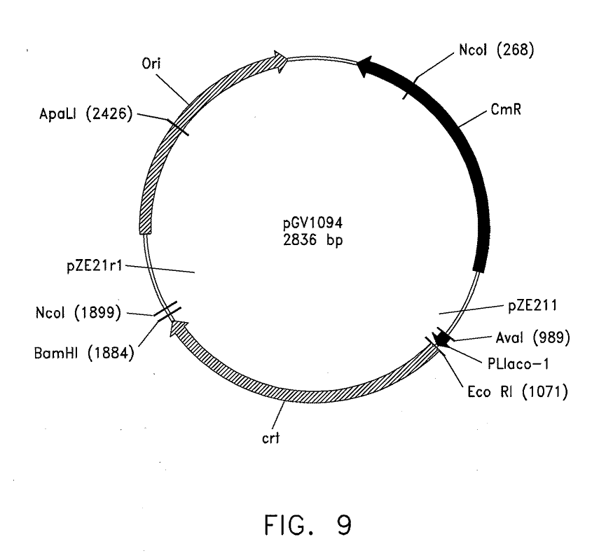

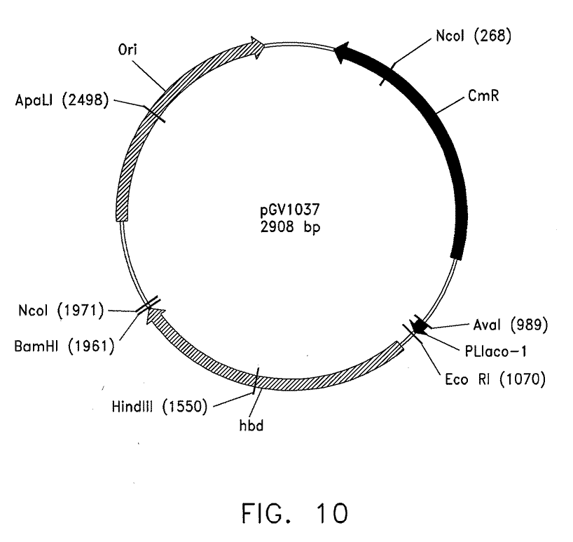

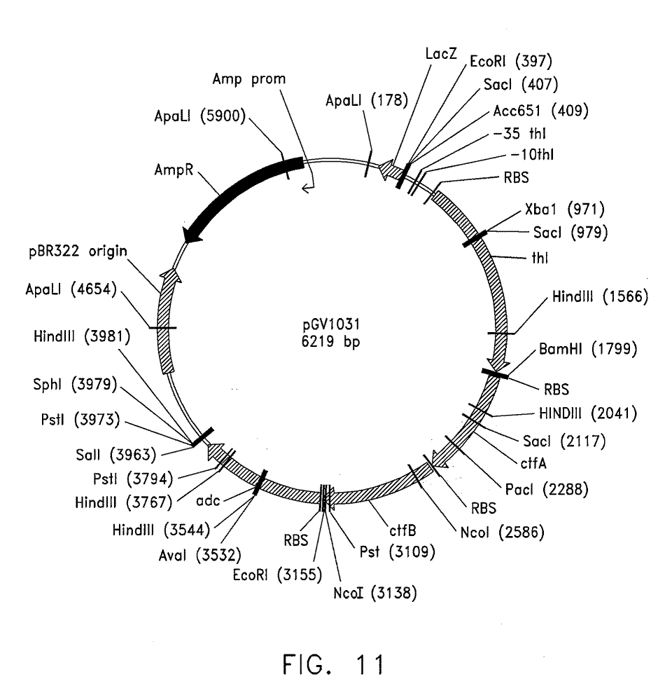

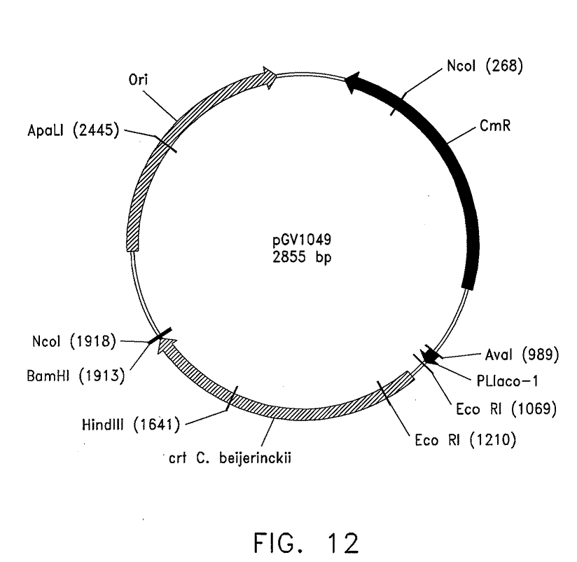

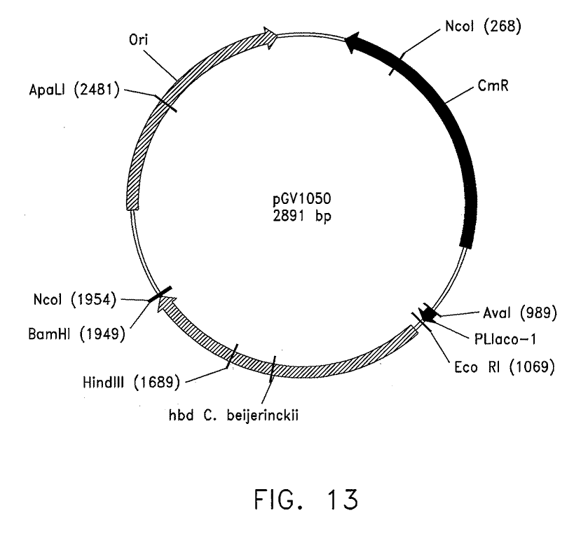

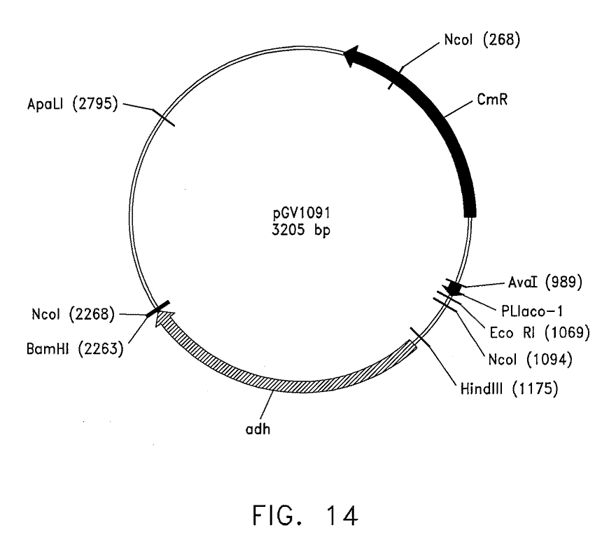

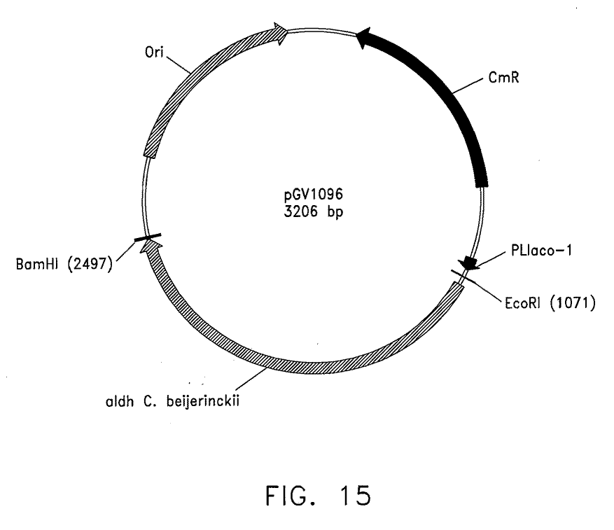

The butanol amounts produced over time by the best two producers, transformants Gevo1099 and Gevo1102, relative to the isolates transformed with only the empty vectors, Gevo1110 and Gevo1111 are shown below (FIG. 6). Gevo 1099 and Gevo 1102 displayed an increase in butanol production over time with the butanol concentration increasing from 123 μM to 313 μM and 57 μM to 317 μM, respectively, from 24 to 72 hours post inoculation.

### Example 5

### Cloning and Expression of E. coli Pyruvate Dehydrogenase Subunits in Saccharomyces cerevisiae

The purpose of this Example is to describe how to clone aceE, aceF, and lpdA genes from E. coli, which together comprise the three subunits of the enzyme pyruvate dehydrogenase (PDH) as found in E. coli. The three genes were amplified from genomic DNA using PCR. This Example also illustrates how the protein products of these three genes were expressed in a host organism, Saccharomyces cerevisiae.

The lpdA gene from E. coli was amplified by PCR using E. coli genomic DNA as a template. To amplify specifically lpdA, the primers Gevo-610 and Gevo-611 were used; other PCR amplification reagents were supplied in manufacturer's kits, for example, KOD Hot Start Polymerase (Novagen, Inc., catalog #71086-5), and used according to the manufacturer's protocol. The forward and reverse primers incorporated nucleotides encoding SalI and XhoI restriction endonuclease sites, respectively. The resulting PCR product was digested with SalI and XhoI and cloned into pGV1103, yielding pGV1334. The inserted lpdA DNA was sequenced in its entirety.

The aceE and aceF genes from E. coli were inserted into pGV1334 using an approach similar to that described above. The aceE gene was amplified from E. coli genomic DNA using the primers Gevo-606 and Gevo-607, digested with SalI+XhoI, and cloned into the vector pGV1334 cut with SalI+XhoI, yielding pGV1379. The aceE insert was sequenced in its entirety. To obtain a plasmid with a different selectable prototrophic marker suitable for S. cerevisiae expression, the aceE insert was cloned out of pGV1379 as a SalI+XhoI fragment and cloned into SalI+XhoI cut pGV1104 yielding pGV1603.

The aceF gene was amplified from E. coli genomic DNA using the primers Gevo-653 and Gevo-609. The resulting 1.9 kb product was digested with SalI+XhoI and cloned into the vector pGV1334, cut with the same enzymes, yielding pGV1380. The aceF insert was sequenced in its entirety. To obtain a plasmid with a different selectable marker suitable for S. cerevisiae expression, the aceF insert was cloned out of pGV1380 and cloned into pGV1105, yielding pGV1604.

To express these proteins in S. cerevisiae, the S. cerevisiae strain Gevo1187 (CEN.PK) was transformed with any combination of pGV1334, pGV1603, and pGV1604, and transformants selected on appropriate dropout media as described in Example 3. As a control, cells were transformed with the corresponding empty vectors—pGV1103, pGV1104, and pGV1105, respectively. Cultures grown from transformants were assayed for LpdA, AceE, or AceF expression by preparing crude yeast protein extracts and analyzing them by Western blotting (based on detecting the Myc epitope present in each protein) as described in Example 2.

### Example 6

### Cloning of S. cerevisiae PDH Subunits from Genomic DNA, Modified to Remove Endogenous Mitochondrial Targeting Sequences, and their Expression in S. cerevisiae Cells

In most eukaryotes, the pyruvate dehydrogenase (PDH) complex is localized inside the mitochondria. The various proteins comprising PDH are directed to enter the mitochondria by virtue of their containing, in their N-terminal region, around 20-40 amino acids commonly known as a mitochondrial targeting sequence. The presence of such a sequence can be determined experimentally or computationally (e.g. by the program MitoProt: http://mips.gsf.de/cgi-bin/proj/medgen/mitofilter). Successful mitochondrial import of the protein is followed by specific proteolytic cleavage and removal of the targeting sequence, resulting in a “cleaved” imported form. It is well known that removing such a sequence from a protein by genetic alteration of its coding sequence causes that protein to become unable to transit into the mitochondria. Thus, an attractive strategy to redirect a normally mitochondrial protein into the cytosol involves expressing only that portion of the gene encoding the “cleaved” portion of the protein remaining after mitochondrial import and subsequent protease cleavage.

The purpose of this Example is to describe the cloning of several of the genes comprising the S. cerevisiae pyruvate dehydrogenase complex, and the expression and detection of these genes in a culture of S. cerevisiae cells.

Several of the genes that encode subunits of PDH were cloned by PCR, using essentially the procedure described in Example 5, except the template was S. cerevisiae genomic DNA. The S. cerevisiae gene to be amplified and the corresponding primers that were used are shown in Table 1.

To generate genes encoding proteins predicted to be localized in the cytosol, the first primer listed in each pair of primers (listed in Table 1) was designed to amplify a region of each gene downstream of the portion predicted to encode the mitochondrial targeting sequence. The resulting PCR products were cloned into the vector pGV1103 using unique restriction enzyme sites encoded in the primers used to amplify each gene, yielding the plasmids listed in Table 2. Each insert was sequenced in its entirety. To test for expression of each gene, S. cerevisiae strain Gevo1187 (CEN.PK) was transformed singly with each of pGV1381, pGV1383, pGV1384, or pGV1385, following essentially the procedure as described in Example 3, and selecting HIS+ colonies on SC-his defined dropout media. Protein expression was assayed by lysate preparation and Western blotting (to detect the Myc tag present on each protein) as described (Example 2).

### Example 7

### Prophetic. Cloning and Expression of the S. cerevisiae Subunit LPD1 and its Expression in S. cerevisiae Cells

This prophetic Example describes how to clone the gene LPD1 from S. cerevisiae genomic DNA by PCR, and how to detect expression of LPD1 in a host S. cerevisiae cell.

The open reading of Lpd1 lacking those nucleotides predicted to encode the mitochondrial targeting sequence are amplified using the primers Gevo-658 plus Gevo-659 in a PCR reaction, essentially as described in Example 5. A 1.5 kb product is digested with XhoI+BamHI and cloned into pGV1103 cut with the same restriction enzymes. The resulting clone, pGV1103-lpd1, is transformed into Gevo 1187 and resultant colonies are selected by HIS+ prototrophy, essentially as described in Example 3. A culture of cells containing pGV1103-lpd1 is grown and LPD1 expression is detected by harvesting of cells followed by Western blotting (for the Myc tag present on the protein) essentially as described in Example 2.

### Example 8

### Prophetic. Cloning of E. coli PDH Subunits and their Expression in K. lactis

Certain yeasts, especially those known as “Crabtree negative”, offer distinct advantages as a production host. Unlike Crabtree-positive strains (e.g. Saccharomyces cerevisiae) which ferment excess glucose to ethanol under aerobic conditions, Crabtree-negative strains, such as those of the genus Kluyveromyces, will instead metabolize glucose via the TCA cycle to yield biomass. Consequently, Crabtree-negative yeasts are tolerant of inactivation (during aerobic growth) of the so-called PDH-bypass route of glucose dissimilation, which can occur, for example, by deletion of the KIPDC1 gene.

The following prophetic Example describes how to clone the genes encoding the three subunits of E. coli PDH into vectors suitable for expression in the yeast Kluyveromyces lactis, and also how to detect the expression of those genes.

The E. coli genes lpdA, aceE, and aceF are amplified by PCR as described in Example 5. Resulting PCR products are digested with SalI+XhoI and cloned into the vectors pGV1428, pGV1429, and pGV1430, respectively, each cut SalI+XhoI. These steps yield the plasmids pGV1428-lpdA, pGV1429-aceE, and pGV1430-aceF. Each insert is sequenced in its entiretyA strain of K. lactis (e.g Gevo 1287) is transformed with one or any combination of these plasmids according to known methods (e.g. Kooistra R, Hooykaas P J, Steensma H Y. (2004) Yeast. 15; 21(9):781-92), and resultant colonies are selected by appropriate prototrophies. Cultures grown from transformants are assayed for LpdA, AceE, or AceF expression using crude yeast protein extracts and Western blot analysis (based on detecting the Myc epitope present in each protein) as described in Example 2.

### Example 9

### Prophetic. Measurement of PDH Activity in Cells Overexpressing PDH Subunits

The purpose of this Example is to describe how PDH activity can be measured by means of an in vitro assay.

A method to quantitate PDH activity in a cell lysate Is described in the literature: (Wenzel T J, et al. (1992). Eur J Biochem 209(2):697-705.) This method utilizes a lysate derived from a cellular fraction enriched in mitochondria. A different embodiment of this method utilizes, as a source of PDH, cell lysates obtained from whole cells. Such lysates are prepared as described previously (Example 2). Another embodiment of this assay method uses a cell lysate derived from a cellular fraction highly enriched for cytosolic (non-mitochondrial) proteins. This biochemical fractionation will reduced the contribution of endogenous mitochondrial PDH in the assay. Methods to prepare such enriched lysates are commerically available and well-known to those skilled in the art; (e.g. Mitochondrial/Cytosol Fractionation Kit, BioVision, Inc., Mountain View, Calif.).

In another embodiment, PDH activity is immunopurified from cells by virtue of the presence of a Myc epitope tag encoded in one or more of the expression plasmid. Methods to immunopurify epitope-tagged proteins are well-known to those skilled in the art (e.g. Harlow and Lane, Antibodies: A Laboratory Manual, (1988) CSHL Press). The immunopurified PDH complex is thus distinct from endogenous complexes and serves as the source of activity in the aforementioned PDH in vitro assay.

### Example 10

### Prophetic. Measurement of Increased Intracellular acetyl-CoA in Cells Overexpressing PDH

The purpose of this example is to describe how intracellular levels of acetyl-CoA, a product of PDH, can be measured in a population of cultured yeast cells.

To measure intracellular acetyl-CoA, those yeast transfromants carrying appropriate plasmid combinations necessary to express the complete set of PDH genes (e.g. pGV1334, pGV1603, and pGV1604) will be assessed for cellular acetyl-CoA levels in comparison to the vector-only control transformants (e.g. pGV1103, pGV1104, and pGV1105). Yeast cells are grown to saturation in appropriate defined dropout media (e.g. SC-His, -Leu, -Trp) in shake flasks. The optical density (OD600) of the culture is determined and cells pelletted by centrifugation at 2800×g for 5 minutes. The cells are lysed using a bead beater and the lysates are utilized for protein determination and analysis for acetyl-CoA determination with established methods (Zhang et al, Connection of Propionyl-CoA Metabolism to Polyketide Biosynthesis in Aspergillus nidulans. Genetics, 168:785-794).

### Example 11

### Prophetic. Co-Expression of E. coli PDH Subunit Genes and a Butanol Production Pathway in S. cerevisiae

The purpose of this Example is to describe how genes encoding the E. coli PDH subunits will be co-expressed with those genes comprising a butanol production pathway, in the host Saccharomyces cerevisiae. Co-expressing PDH with a butanol production pathway will increase the yield of butanol produced relative to merely expressing the butanol pathway without heterologously expressed functional PDH in the cytosol.

The cloned genes lpdA, aceE and aceF (see Example 5) are subcloned into butanol pathway gene plasmids, specifically pGV1208, pGV1209 and pGV1213 (Table 2). To do this, pGV1334, pGV1603 and pGV1604 are each digested with the restriction enzymes EcoICRI plus XhoI, and the resulting released insert is ligated into pGV1208, pGV1209 and pGV1213 that is digested with BamHI, the overhang filled in by Klenow DNA polymerase, and then digested with XhoI, all using standard molecular biology methods (Sambrook, J. Fritsch, E. F., Maniatis, T., 1989). These steps yield pGV1208-lpdA, pGV1209-aceE and pGV1213-aceF, respectively. The resulting plasmids are transformed along with pGV1227 into Gevo 1187 and selected for HIS, LEU, TRP and URA prototrophy, all essentially as described in Example 3. Strains transformed with the parental plasmids pGV1208 plus pGV1209 plus pVG1213 plus pGV1227 are used as controls, to assess the affect of PDH co-expression on butanol production. Production of butanol is performed as described in Example 4. The expected n-butanol yield is greater than 10%.

### Example 12

### Prophetic. Generation of a Form of PDH that is Functional Under Anaerobic Conditions, or Under Conditions of Excess NADH

The purpose of this Example is to describe the isolation of a mutant form of PDH which is active anaerobically, or is active in the presence of a high [NADH]/[NAD+] ratio relative to the ratio present during normal aerobic growth. Such a mutant form of PDH is desirable in that it may allow for continued PDH enzymatic activity even under microaerobic or anaerobic conditions.

Methods to obtain and identify altered versions of PDH that permit microaerobic or anaerobic activity have been described previously: (Kim, Y. et al. (2007). Appl. Environm. Microbiol., 73, 1766-1771; U.S. patent application Ser. No. 11/949,724, which is incorporated herein in its entirety).

### Example 13

### Prophetic. Co-Expression of E. coli PDH Subunit Genes and a Butanol Production Pathway in a S. cerevisiae Strain with Reduced or Absent Pyruvate Decarboxylase Activity

The purpose of this Example is to describe how genes encoding the E. coli PDH subunits are co-expressed with genes comprising a butanol production pathway, in a host Saccharomyces cerevisiae strain with reduced or absent pyruvate decarboxylase (PDC) activity. Both PDC and PDH utilize and therefore compete for available pyruvate pools. Whereas the product of PDH, acetyl-CoA, can be directly utilized by the butanol pathway, the product of PDC, acetaldehyde, can be further reduced to ethanol (via alcohol dehydrogenase), an undesired side-product of butanol fermentation, or can be converted to acetyl-CoA via the concerted action of acetaldehyde dehydrogenase plus acetyl-CoA synthase. Thus, reducing or eliminating PDC activity will increase the yield of butanol from pyruvate in a cell also overexpressing functional PDH in the cytosol.

Generation of a pdc-Strain of S. cerevisiae

Strains of S. cerevisiae having reduced or absent PDC activity are described in the literature (e.g., Flikweert, M. T., et al., (1996). Yeast 15; 12(3):247-57; Flikweert M T, et al., (1999). FEMS Microbiol Lett. 1; 174(1):73-9; van Maris A J, et al., (2004) Appl Environ Microbiol. 70(1):159-66. and are well-known to those skilled in the art. In one embodiment, a strain of S. cerevisiae lacking all PDC activity has the genotype pdc1Δ pdc5Δ pdc6Δ. Such strains lacks detectable PDC activity and are unable to grow on glucose as a sole carbon source, but can live when the growth media is supplemented with ethanol or acetate as an alternative carbon source. In another embodiment, a derivative of this strain has been evolved to grow on glucose, a convenient and commonly used carbon source. A third embodiment of a strain with greatly reduced PDC activity is a strain of the relevant genotype pdc2Δ, also described in the literature (Flikweert M T, et al., (1999). Biotechnol Bioeng. 66(1):42-50). Any of these strains can serve as a useful host for the expression of PDH plus a butanol pathway. If necessary, any pdc-mutant strain will be engineered, by means of standard molecular biology and yeast genetic techniques, to make available those auxotrophic markers such that the plasmids pGV1208-lpdA, pGV1209-aceE, and pGV1213-aceF can be selected and stably maintained within a host cell. Such genetic engineering will take place by disruption of the relevant endogenous gene by a URA3-based disruption cassette, with subsequent removal of the URA3 marker by FOA counterselection.

Butanol Production in a PDH-Overexpressing pdc-Strain

The cloned genes lpdA, aceE and aceF (see Example 5) are subcloned into butanol pathway gene plasmids, specifically pGV1208, pGV1209 and pGV1213 (Table 2), essentially as described in Example 11.

The set of plasmids pGV1208-lpdA plus pGV1209-aceE plus pGV1213-aceF plus pGV1227, or the set pGV1208 plus pGV1209 plus pGV1213 plus pGV1227 as a control, are transformed into the appropriate pdc-mutant yeast strain and resulting colonies grown in liquid culture. Production of butanol is performed as described in Example 4. The expected n-butanol yield is greater than 50%.

It is likely that strains with diminished or absent PDC activity will exhibit a pronounced growth defect, and therefore may have to be supplemented with an additional carbon source (e.g. acetate or ethanol). Since the defect in growth in pdc-S. cerevisiae arises from their lack of cytoplasmic pools of acetyl-CoA, it is expected that successful expression of PDH in the cytosol will generate sufficient acetyl-CoA to rescue this growth defect. Such restoration of growth can serve as a useful in vivo readout of PDH activity in the cytosol.

### Example 14

### (Prophetic). pfl (Pyruvate Formate Lyase) and FDH1 (Formate Dehydrogenase) Expression in Saccharomyces cerevisiae

Cloning of E. coli pflB (Inactive Pyruvate Formate Lyase) and pflA (Pyruvate Formate Lyase Activating Enzyme).

For the cloning of Escherichia coli pflB and pflA, genes are amplified using E. coli genomic DNA and pflB_forw, PflB_rev and PflA_forw, PflA_rev primers, respectively. For the cloning of the Candida boidinii FDH1 (Cb-FDH1) gene, genomic DNA of Canida boidinii is used with fdh_forw and fdh_rev primers. Utilizing the restriction sites, SalI and EcoRI incorporated into the forward and reverse gene amplification primers, respectively, the amplified DNA is ligated onto SalI and EcoRI digested pGV1103, pGV1104 and pGV1102 yielding pGV1103pflA, pGV1104pflB and pGV1002fdh1. The proteins expressed from the resulting plasmids are tagged with myc, myc and HA tags, respectively.

The resulting plasmids (pGV1103pflA, pGV1104pflB and pGV1002fdh1) and vectors (pGV1103, pGV1104 and pGV1102) are utilized to transform yeast strain Gevo 1187 as indicated by example 3 to yield PflA, PflB, Fdh1 expressing (PFL+) and control (PFL−) transformants. Both sets of transformants are chosen by selection for HIS, TRP and URA prototrophy.

The resulting trasformants are evaluated for PflA, PflB and Cb-Fdh1 expression using crude yeast protein extracts and western blot analysis as described in Example 2.

Those yeast transfromants verified to express all three proteins are assessed for cellular acetyl-CoA levels in comparison to the vector only control transformants. For this, PFL+ and PFL− cells are grown in SC-ura, his, trp medium in shake flask format. The optical density (OD600) of the culture determined and cells pelletted by centrifugation at 2800 xrcf for 5 minutes. The cells are lysed using a bead beater and the lysates are utilized for protein determination and analysis for acetyl-CoA determination with established methods (Zhang et al, Connection of Propionyl-CoA Metabolism to Polyketide Biosynthesis in Aspergillus nidulans. Genetics, 168:785-794). Acetyl-CoA amounts are assessed per mg of cellular total protein.

To evaluate the effect of PflA, PflB and Fdh1 expression on n-butanol production, pflA, pflB and Cb-FDH1 are subcloned into butanol pathway gene containing pGV1208, pGV1209 and pGV1213 (Table 1). For this, pGV1103pflA, pGV1104pflB and pGV1002fdh1 are digested with EcoICRI+XhoI restriction enzymes and ligated into pGV1208, pGV1209 and pGV1213 digested with BamHI (and subsequently blunt ended with Klenow fill-in)+XhoI using standard molecular biology methods (Sambrook, J. Fritsch, E. F., Maniatis, T., 1989) to yield pGV1208PflA, pGV1209PflB and pGV1213Fdh1. The resulting plasmids along with pGV1227 are transformed into Gevo 1187 and selected for His, Leu, Trp and Ura prototrophy. Gevo 1110 and Gevo 1111 are used as control isolates (Table 1). Production of butanol is performed as described in Example 4. The expected n-butanol yield is greater than 10%.

### Example 15

### (Prophetic) PflA, PflB and Fdh1 Expression in Saccharomyces cerevisiae with Reduced or Absent Pyruvate Decarboxylase Activity

Cloning of E. coli pflB (inactive Pyruvate formate lyase) and pflA (Pyruvate formate lyase activating enzyme) and Cb-FDH1 is done as described in Example 14.

The resulting plasmids (pGV1103pflA, pGV1104pflB and pGV1002fdh1) and vectors (pGV1103, pGV1104 and pGV1102) are utilized to transform S. cerevisiae (relevant genotype: ura3, trp1, his3, leu2, pdc1, pdc5, pdc6) yeast strain as indicated by example 3 to yield PflA, PflB, Cb-Fdh1 expressing (PFL+) and control (PFL−) transformants. Both sets of transformants are chosen by selection for HIS, TRP and URA prototrophy.

The resulting trasformants will be evaluated for PflA, PflB and Fdh1 expression using crude yeast protein extracts and western blot analysis as described in Example 2.

Those yeast transfromants verified to express all three proteins are assessed for cellular acetyl-CoA levels in comparison to the vector only control transformants as described in Example 14.

To evaluate the effect of expressing PflA, PflB and Fdh1 on n-butanol production, pGV1208PflA, pGV1209PflB and pGV1213Fdh1 along with pGV1227 are transformed into S. cerevisiae (MAT A, ura3, trp1, his3, leu2, pdc1, pdc5, pdc6) and selected for His, Leu, Trp and Ura prototrophy. Gevo 1110 and 1111 are used as control isolates (Table 1). Production of butanol is performed as described in Example 4. The expected n-butanol yield is greater than 50%.

### Example 16

### (Prophetic) Pfl and Fdh1 Expression in Saccharomyces cerevisiae with Reduced or Absent ADH1 Activity

Cloning of E. coli pflB (inactive Pyruvate formate lyase) and pflA (Pyruvate formate lyase activating enzyme)

Cloning of E. coli pflB (inactive Pyruvate formate lyase) and pflA (Pyruvate formate lyase activating enzyme) and Cb-FDH1 is done as described in Example 14.

The resulting plasmids (pGV1103pflA, pGV1104pflB and pGV1002fdh1) and vectors (pGV1103, pGV1104 and pGV1102) are utilized to transform yeast strain Gevo 1253 (adh1Δ) as described in Example 3 to yield PflA, PflB, Fdh1 expressing (PFL+) and control (PFL−) transformants. Both sets of transformants are chosen by selection for HIS, TRP and URA prototrophy.

The resulting trasformants will be evaluated for PflA, PflB and Fdh1 expression using crude yeast protein extracts and western blot analysis as described in Example 2.

Those yeast transfromants verified to express all three proteins are assessed for cellular acetyl-CoA levels in comparison to the vector only control transformants as described in Example 14.

To evaluate the effect of overexpressing PflA, PflB and Fdh1 on n-butanol production, pGV1208PflA, pGV1209PflB and pGV1213Fdh1 along with pGV1227 are transformed into Gevo 1253 and selected for His, Leu, Trp and Ura prototrophy. Gevo 1110 and 1111 are used as control isolates (Table 1). Production of butanol is performed as described in Example 4. The expected n-butanol yield is greater than 10%.

### Example 17

### Cloning of PDC1 Gene from S. cerevisiae, and its Overexpression in S. cerevisiae

The purpose of this example is to describe the cloning of a gene encoding pyruvate decarboxylase under the control of a constitutively active promoter, and to describe the expression of such a gene in an S. cerevisiae host cell.

The complete PDC10RF was amplified from S. cerevisiae genomic DNA using primers Gevo-639 plus Gevo-640 in a PCR reaction that was carried out essentially as described (Example 5). The resulting 1.7 kb product was digested with XhoI+BamHI and ligated into the vector pGV1106, which was cut SalI+BamHI, yielding pGV1389 (see Table 2). The insert was sequenced in its entirety).

To overexpress Pdc1 in S. cerevisiae, the S. cerevisiae strain Gevo1187 (CEN.PK) was transformed with pGV1389, and transformants selected on SC-ura dropout media as described in Example 3. Cultures grown from transformants were assayed for Pdc1 expression using crude yeast protein extracts and Western blot analysis (based on detecting the Myc epitope present in the recombinant expressed protein) as described in Example 2.

### Example 18

### Cloning to Permit Inducible Expression of a Pyruvate Decarboxylase Gene

The constitutive expression of a gene, for example pyruvate decarboxylase, may be undesirable at certain points during a culture's growth, or may exert an unexpected metabolic or selective pressure on those overexpressing cells. Thus, there is a need to employ a system of regulated gene expression, whereby a gene of interest may be expressed chiefly at an optimal time to maximize culture growth as well as performance in a subsequent fermentation.

The purpose of this example is to describe the cloning of a gene encoding the enzyme pyruvate decarboxylase under the control of an inducibly-regulated promoter, and to describe the expression of such a gene in an S. cerevisiae host cell.

The PDC1 ORF present in pGV1389 (see Example 19) was released as an XbaI+BamHI fragment and cloned into the vector pGV1414 which had been digested AvrII+BamHI, yielding vector pGV1483. Vector pGV1483 (Table 2) thus features the S. cerevisiae MET3 gene promoter (SEQ ID NO:177) driving the expression of the PDC1 gene. The MET3 promoter is transcriptionally silent in the presence of methionine but becomes active when methionine levels fall below a certain threshold. The plasmid pGV1483 is transformed into Gevo 1187 and resulting transformants are identified by selection on SC-ura media, as described in Example 3. Cultures of Gevo 1187 carrying pGV1483 are grown and assayed for PDC1 expression essentially as described in Example 2.

In another embodiment of this Example, the PDC1 gene is expressed under the control of the S. cerevisiae copper-inducible CUP1 gene promoter (SEQ ID NO:178). First, the CUP1 gene promoter was amplified by PCR from S. cerevisiae genomic DNA using primers in a reaction essentially as described in (Example 5). The PCR product was digested SacI+SalI and inserted into pGV1106 that was cut SacI+SalI, yielding pGV1388. The inserted CUP1 promoter sequence was sequenced in its entirety. Next, an XbaI+BamHI fragment containing the PDC1 gene from pGV1389 is inserted into the AvrII+BamHI-digested pGV1388, yielding pGV1388-PDC1. Plasmid pGV1388-PDC1 is transformed into Gevo 1187, as described in Example 3, and transformants are identified on SC-ura defined media lacking copper. Cultures of transformed cells are grown in SC-ura media without copper supplementation until they reach an OD600 of >0.5, at which time copper sulfate is added to a final concentration of 0.5 mM. The cultures are grown for an additional 24 h to 48 h, as desired, and then assayed for expression of Pdc1 by Western blotting, essentially as described (Example 2).

### Example 19

### Prophetic. An In Vitro Assay to Measure PDC Activity Produced in a Culture of Yeast Cells Overexpressing a Pyruvate Decarboxylase Enzyme

The purpose of this Example is to describe an in vitro assay useful for determining the total pyruvate decarboxylase activity present in a cell, and in particular from a population of cells overexpressing a PDC enzyme.

Assays to measure PDC activity from total cell lysates have been described and are well-known to those skilled in the art (Maitra P K & Lobo Z. 1971. J Biol Chem. 25; 246(2):475-88.; Schmitt H D & Zimmermann F K. 1982. J Bacteriol. 151(3):1146-52; Eberhardt et al., (1999) Eur. J. Biochem. 262(1), 191-201).

In another embodiment of this Example, PDC activity generated by expression of PDC as described in Examples 17 and 18 is measured by first immunoprecipitating PDC, using a specific antibody directed against PDC, or using an antibody directed against the Myc epitope tag, which is present in the overexpressed (but not endogenous) PDC as expressed in Examples RF20 and RF21. Methods to specifically immunoprecipitate proteins present in a complex mixture are well-known to those skilled in the art (e.g., Harlow and Lane, 1988, Antibodies: A Laboratory Manual, CSHL Press). The immunoprecipitated PDC complexes then serve as the source of material to be assayed using the aforementioned assays. This method thus allows the specific assay of heterologous, overexpressed PDC.

### Example 20

### Prophetic. Increased Butanol Productivity Resulting from PDC Overexpression in S. cerevisiae that also Contains a Functional Butanol Production Pathway

The purpose of this Example is to illustrate how PDC overexpression increases butanol productivity in a culture of Saccharomyces cerevisiae also expressing a butanol production pathway.

A strain of S. cerevisiae overexpressing a PDC gene has been described previously (van Hoek et al., (1998). Appl Environ Microbiol. 64(6):2133-40). These experiments revealed that (1) endogenous PDC levels in S. cerevisiae, while comprising up to 3.4. % of the total cellular protein, can be further increased by the presence of an overexpression construct; and (2) the fermentative capacity (the maximum specific rate of ethanol production) of PDC-overexpressing cultures at high growth rates was increased relative to that of control strains. These results suggest that overexpression of PDC, under certain growth conditions, will increase the flux through a heterologously supplied butanol production pathway.

To overexpress a PDC gene in the presence of a butanol pathway, the PDC1 gene is excised from pGV1389 by digestion with SpeI, the cut DNA overhang is filled in with Klenow DNA polymerase fragment, and the vector then digested with XhoI. The fragment is inserted into pGV1213 that is digested with BamHI, the cut ends filled in with Klenow enzyme, and then digested with XhoI, yielding plasmid pGV1605. Plasmid pGV1605 or pGV1057 (Mumberg, D., et al. (1995) Gene 156:119-122) is transformed into Gevo 1187 along with plasmids pGV1208, pGV1209, and pGV1213, essentially as described (Example 3) and selected for His, Leu, Trp, and Ura prototrophy. Fermentations are carried out to produce butanol, which is measured as described (Example 4). The inclusion of pGV1605 results in higher butanol productivity (amount of butanol produced per unit time) than does the inclusion of pGV1057 with plasmids pGV1208, pGV1209, and pGV1213 in the aforementioned fermentations. The expected n-butanol yield is greater than 5%.

### Example 21

### Prophetic. Increased Butanol Productivity Resulting from PDC Overexpression in an S. cerevisiae Cell that has Reduced Alcohol Dehydrogenase Activity and that also Contains a Functional Butanol Production Pathway

The purpose of this Example is to demonstrate how enhanced butanol productivity is obtained by overexpressing a PDC gene in the presence of a butanol production pathway, in a yeast strain deficient in alcohol dehydrogenase (ADH) activity.

Acetaldehyde generated from pyruvate by PDC has two main fates: it can be further metabolized to acetyl-CoA by the action of acetaldehyde dehydrogenase and acetyl-CoA synthase, where it may then be a useful substrate for a butanol synthetic pathway; or, it can be further metabolized by a reductive process to ethanol, by the action of an alcohol dehydrogenase (ADH) enzyme. Therefore, diminishing or removing ADHs, especially those ADH enzymes with a preference for acetaldehye, would reduce or eliminate this undesirable route of acetaldehyde dissimilation and increase available acetyl-CoA pools a butanol pathway.

Plasmids pGV1208, pGV1209, pGV1213, and pGV1605 are simultaneously co-transformed into strain Gevo 1187, which has the relevant genotype ADH1⁺, or into strain Gevo1266, which has the relevant genotype adh1Δ. Transformed colonies are selected for His, Leu, Trp, and Ura prototrophy, essentially as described in Example 3. Fermentations are carried out to produce butanol, which is measured as described in Example 4. The expected n-butanol yield is greater than 10%. Strain Gevo1266 (adh1Δ) exhibits an improved yield of butanol over a parallel fermentation carried out in strain Gevo 1187 (ADH1⁺).

### Example 22

### Prophetic. Increased Butanol Yield Resulting from PDC Overexpression in a K. lactis Cell with Reduced Alcohol Dehydrogenase Activity and Expressing a Functional Butanol Production Pathway

The purpose of this Example is to describe the production of butanol in a K. lactis strain with greatly reduced or absent ADH activity. It is predicted that expression of a butanol pathway in such a strain will yield significantly greater yields of butanol per input glucose than would the expression of a butanol pathway in a strain with ADH activity.

Generation of a Kluyveromyces lactis strain with reduced alcohol dehydrogenase activity.

Methods to transform cells of and disrupt genes in Kluyveromyces lactis—i.e., to replace a functional open reading frame with a selectable marker, followed by the subsequent removal of the marker—have been described previously (Kooistra R, Hooykaas P J, Steensma H Y. (2004) Yeast. 15; 21(9):781-92). Kluyveromyces lactis has four genes encoding ADH enzymes, two of which, KIADH1 and KIADH2, are localized to the cytoplasm. A mutant derivative of K. lactis in which all four genes were deleted (called K. lactis adh⁰) has been described in the literature (Saliola, M., et al., (1994) Yeast 10(9):1133-40), as well as the culture conditions required to ideally grow this strain. An alternative version of this approach employs using a marker conferring resistance to the drug G418/geneticin, for example as provided by the kan gene. Such an approach is useful in that it leaves the URA3 marker available for use as a selectable marker in subsequent transformations.

Expression of a Butanol Expression Pathway in an adh0 strain of K. lactis

Plasmids pGV1208, pGV1209, pGV1213, and pGV1605 are simultaneously co-transformed into strain Gevo 1287, which is ADH⁺, or into an adh⁰ strain. Transformed colonies are selected for His, Leu, Trp, and Ura prototroph. Fermentations are carried out to produce butanol, which is measured as described in Example 4. The expected n-butanol yield is greater than 10%. Strain Gevo1287 produces significantly more butanol than does the parallel fermentation carried out in the otherwise isogenic adh⁰ strain.

### Example 23

### (Prophetic). ALD6 Over-Expression in Saccharomyces cerevisiae

To clone the ALD6 gene of S. cerevisiae, a two step fusion PCR method was employed that eliminated an internal SalI restriction enzyme site to facilitate subsequent molecular biology manipulations. Two overlapping PCR products that spans the sequence of the S. cerevisiae ALD6 gene were generated using primers pairs Gevo-643 & Gevo-644 and Gevo-645 & Gevo-646 with S. cerevisiae genomic DNA as the template. The resulting PCR fragment was digested with SalI+BamHI and ligated into similarly restriction digested pGV1105 and pGV1101 to yield pGV1321 and pGV1326. Subsequently, ALD6 was subcloned by digestion of pGV1321 and pGV1326 with EcoICRI+XhoI and ligation into BamHI (and subsequently blunt ended by Klenow fill-in)+XhoI digested pGV1209 and pGV1208 to yield pGV1339 and pGV1399, respectively.

The resulting plasmids (pGV1339 and pGV1399) and vectors (pGV1105 and pGV1101) are utilized to transform yeast strain Gevo 1187 as described in Example 3 to yield ALD6 over-expressing (“Ald6+”) or control transformants, respectively. Both sets of transformants are chosen by selection for TRP and LEU prototrophy appropriate dropout medium.

The resulting trasformants are evaluated for Ald6 expression using crude yeast protein extracts and western blot analysis as described in Example 2.

Those yeast transfromants verified to express Ald6 proteins are assessed for enhanced acetaldehyde dehydrogenase activity in comparison to the vector only control transformants. For this, Ald6+ and control cells are grown in appriate dropout medium in shake flasks. The optical density (OD600) of the culture is determined and cells pelletted by centrifugation at 2800×g for 5 minutes. The cells are lysed using a bead beater and the lysates are utilized for protein determination and analysis for aldehyde dehydrogenase activity using established methods (for example, Van Urk et al, Biochim. Biophys. Acta, 191:769).

To evaluate the effect of overexpressing Ald6 on n-butanol production, pGV1339 is transformed into Gevo 1187 along with pGV1208, pGV1227 and pGV1213 and selected for His, Leu, Trp and Ura prototrophy. Gevo 1110 and 1111 are used as control isolates (Table 1). Production of butanol is performed as described in Example 4. The expected n-butanol yield is greater than 5%.

### Example 24

### (Prophetic). Ald6 Overexpression in a Saccharomyces cerevisiae with No Alcohol Dehydrogenase I Activity (adh1Δ)

Cloning of ALD6 gene is carried out as described in Example 23.

The resulting plasmids (pGV1339 and pGV1399) and vectors (pGV1100 and pGV1101) are utilized to transform yeast strain Gevo 1253 as indicated by example 3 to yield Ald6+ overexpressing and control transformants, respectively. Both sets of transformants are chosen on appropriate dropout medium.

The resulting trasformants will be evaluated for Ald6 expression using crude yeast protein extracts and western blot analysis as described in Example 2.

Those yeast transfromants verified to express Ald6 proteins will be assessed for enhanced acetaldehyde dehydrogenase activity as described in Example 23.

To evaluate the consequence of the overexpression of on n-butanol production, pGV1339 will be transformed into Gevo 1253 along with pGV1209, pGV1227 and pGV1213 and selected for His, Leu, Trp and Ura prototrophy. Gevo 1110 and 1111 are used as control isolates (Table 1). Production of butanol is performed as described in Example 4. The expected n-butanol yield is greater than 10%.

### Example 25

### (Prophetic). Overexpression of an acetyl-CoA Synthase Gene in Saccharomyces cerevisiae

The purpose of this Example is to describe the cloning of a gene encoding acefyl-CoA synthase activity, and the expression of such a gene in a host S. cerevisiae cell. Specifically, either or both of the S. cerevisiae genes ACS1 or ACS2 encode acetyl-CoA synthase activity.

For the cloning of ACS1 and ACS2 genes, S. cerevisiae genomic DNA was utilized as template with Primers Gevo-479 & Gevo-480 (ACS1) and Gevo-483 & Gevo-484 (ACS2), each set containing SalI and BamHI restriction sites in the forward and reverse primers, respectively. The resulting PCR fragment was digested with SalI+BamHI and ligated into similarly restriction digested pGV1101 and pGV1102 to yield pGV1262 and pGV1263. Subsequently, ACS1 and ACS2 were subcloned by digestion of pGV1262 and pGV1263 with EcoICRI+XhoI and ligation into BamHI (and subsequently blunt ended with Klenow fill-in)+XhoI digested pGV1213 to yield pGV1319 and pGV1320.

The resulting plasmids, pGV1262 and pGV1263, and vectors pGV1101 and pGV1102 are utilized to transform yeast strain Gevo 1187 as described in Example 3 to yield ACS1+, ACS2+ overexpressing and control transformants, respectively. Both sets of transformants are chosen by selection for LEU, URA prototrophy. The transformants are evaluated for Acs1 or Acs2 expression using crude yeast protein extracts and western blot analysis as described in Example 2.

Those yeast transfromants verified to express Acs1 or Acs2 proteins are assessed for enhanced Acetyl-CoA synthase activity in comparison to the vector only control transformants. For this, ACS1+ or ACS2+ and control cells are grown in SC-LEU, URA medium in shake flask format. The optical density (OD600) of the culture determined and cells pelletted by centrifugation at 2800×rcf for 5 minutes. The cells are lysed using a bead beater and the lysates are utilized for protein determination and analysis for Acetyl-CoA synthase activity using established methods (Van Urk et al, Biochim. Biophys. Acta, 191:769).

To evaluate of the effect of Acs1 or Acs2 overexpression on n-butanol production, pGV1319 and 1320 will be transformed into Gevo 1187 along with pGV1208, pGV1209 and pGV1227 and selected for His, Leu, Trp and Ura prototrophy. Gevo 1110 and 1111 are used as control isolates (Table 1). Production of butanol is performed as described in Example 4. The expected n-butanol yield is greater than 5%.

### Example 26

### (Prophetic). Overexpression of an acetyl-CoA Synthase in Saccharomyces cerevisiae Cell with No Alcohol Dehydrogenase I Activity (adh1A)

Cloning of ACS1 and ACS2 genes of S. cerevisiae are as described in Example 25.

The resulting plasmids, pGV1262 and pGV1263, and vectors pGV1101 and pGV1102 are utilized to transform yeast strain Gevo 1253 as indicated by example 3 to yield ACS1+, ACS2+ and overexpressing and control transformants, respectively. Both sets of transformants are chosen by selection for LEU, URA prototrophy. The trasformants are evaluated for Acs1 or Acs2 expression using crude yeast protein extracts and Western blot analysis as described in Example 25.

Those yeast transformants verified to express Acs1 or Acs2 proteins are assessed for enhanced Acetyl-CoA synthase activity as described in Example 26.

To evaluate of the effect of overexpressing Acs1 or Acs2 on butanol production, pGV1319 and 1320 will be transformed into Gevo 1253 along with pGV1208, pGV1209 and pGV1227 and selected for His, Leu, Trp and Ura prototrophy. Gevo 1110 and 1111 are used as control isolates (Table 1). Production of butanol is performed as described in Example 4. The expected n-butanol yield is greater than 5%.

### Example 27

### (Prophetic). ALD6, ACS1 and ACS2 Overexpression in Saccharomyces cerevisiae

ALD6, ACS1 and ACS2 genes are cloned as described above in Examples 23 and 25.

The resulting plasmids pGV1321 and pGV1262 or pGV1263 and vectors pGV1105 and pGV1102 are utilized to transform yeast strain Gevo 1187 as indicated by Example 3 to yield ALD6+ACS1+, ALD6+ACS2+ over-expressing and control transformants, respectively. Both sets of transformants are chosen by selection for LEU and URA prototrophy.

Transformants ALD6+ACS1+ and ALD6+ACS2+ are assessed for enhanced Acetyl-CoA synthase activity in comparison to the vector-only control transformants. For this, ALD6+ACS1+, ALD6+ACS2+ and control cells are grown in SC-LEU, URA medium in shake flask format and assessed as described in Example 25.

To evaluate the effect of overexpressing Ald6 plus Acs1 or Acs2 results in higher butanol production, Gevo 1187 is transformed with pGV1208, pGV1339, pGV1227 and pGV1319 or 1320 and selected for His, Leu, Trp and Ura prototrophy. Gevo 1110 and 1111 are used as control isolates (Table 1). Production of butanol is assessed as described in Example 4. The expected n-butanol yield is greater than 5%.

### Example 28

### (Prophetic). ALD6 Plus ACS1 or ACS2 Overexpression in Saccharomyces cerevisiae with No Alcohol Dehydrogenase I Activity (adh1Δ)

ALD6, ACS1 and ACS2 genes are cloned as described in Examples 23 and 25.

The resulting plasmids pGV1321 and pGV1262 or pGV1263 and vectors pGV1105 and pGV1102 are utilized to transform yeast strain Gevo 1253 (ΔADH1) as indicated by example 3 to yield ALD6+ACS1+ or ALD6+ACS2+ overexpressing strains or control transformants, respectively. Both sets of transformants are chosen by selection for LEU and URA prototrophy.

Transformants ALD6+ACS1+ or ALD6+ACS2+ are assessed for enhanced Acetyl-CoA synthase activity in comparison to the vector-only control transformants. For this, ALD6+ACS1+ or ALD6+ACS2+ and control cells are grown in SC-LEU, URA medium in shake flask format and assessed as described in Example 25.

To evaluate the effect of overexpressing SALD6 and ACS1 or ACS2 on butanol production, Gevo 1253 is transformed with pGV1208, pGV1339, pGV1227 and pGV1319 or 1320 and selected for HIS, LEU, TRP and URA prototrophy. Gevo 1110 and 1111 are used as control isolates (Table 1). Production of butanol is performed as described in Example 4. The expected n-butanol yield is greater than 10%.

### Example 29

### (Prophetic). Cloning of a Butanol Pathway into Vectors for Expression in a Yeast of the Genus Kluyveromyces

To clone the butanol pathway genes into vectors suitable for expression in the strain Kluyveromyces lactis, hbd, Crt, Thl+TER are released from pGV1208, pGV1209 and pGV1227 by digestion with SacI and NotI restriction digests and cloned into similarly digested pGV1428, 1429 and 1430 to yield pGV1208KI, pGV1209KI and pGV1227KI. To clone ADHE2 into Kluyveromyces lactis, pGV1213 is digested with MluI and SacI and ligated onto similarly digested pGV1431 to yield pGV1213KI. The resulting plasmids, pGV1208KI, pGV1209KI, pGV1227KI and pGV1213KI are transformed into K. lactis (strain Gevo 1287; relevant genotype: MATa, trp1, his3, leu2, ura3) and transformants are selected for TRP, HIS, LEU and URA prototrophy (Kooistra R, Hooykaas P J, Steensma H Y. (2004) Yeast. 15; 21(9):781-92). Production of butanol is performed as described in Example 4.

### Example 30

### (Prophetic). Pyruvate Formate Lyaseand Formate Dehydrogenase I Expression in Kluyveromyces lactis

Cloning of E. coli pflB (Inactive Pyruvate Formate Lyase) and pflA (Pyruvate Formate Lyase Activating Enzyme)

For the cloning of Escherichia coli pflB and pflA, genes are amplified using E. coli genomic DNA and pflB_forw, PflB_rev and PflA_forw, PflA_rev primers, respectively. For the cloning of the Candida boidinii FDH1 gene, genomic DNA of Canida boidinii is used as a template in a PCR reaction with fdh_forw and fdh_rev primers. Utilizing the restriction sites, SalI and EcoRI incorporated into the forward and reverse gene amplification primers, respectively, the amplified DNA is ligated onto Sal I and EcoRI digested pGV1428, pGV1429 and pGV1430 yielding pGV1428pflA, pGV1429pflB and pGV1430fdh1. The proteins expressed from the resulting plasmids are tagged with the myc tags for protein expression studies.

The resulting plasmids (pGV1428pflA, pGV1429pflB and pGV1430fdh1) and vectors (pGV1428, pGV1429 and pGV1430) are utilized to transform yeast strain K. lactis (Gevo 1287; relevant genotype: MatA, trp1, his3, leu2 and ura3) by known methods (Kooistra R, Hooykaas P J, Steensma H Y. (2004) Yeast. 15; 21(9):781-92) to yield PflA, PflB, Cb-Fdh1 expressing (PFL+) and control (PFL−) transformants. Both sets of transformants are chosen by selection for HIS, TRP and LEU prototrophy.

The resulting trasformants are evaluated for PflA, PflB and Fdh1 expression using crude yeast protein extracts and Western blot analysis as described in Example 2.

Those yeast transfromants verified to express all three proteins are assessed for cellular acetyl-CoA levels in comparison to the vector only control transformants. For this, PFL+ and PFL− cells are grown in SC-LEU, HIS, TRP medium in shake flask format. The optical density (OD600) of the culture determined and cells pelletted by centrifugation at 2800 xrcf for 5 minutes. The cells are lysed using a bead beater and the lysates are utilized for protein determination and analysis for acetyl-CoA determination with established methods (Zhang et al, Connection of Propionyl-CoA Metabolism to Polyketide Biosynthesis in Aspergillus nidulans. Genetics, 168:785-794). Acetyl-CoA amounts are assessed per mg of cellular total protein.

To evaluate the effect of the expression of PflA, PflB and Fdh1 on butanol production, the pflA, pflB and Cb-FDH1 are subcloned into butanol pathway gene containing pGV1208KI, pGV1209KI, pGV1227KI and pGV1213KI (Table 1). For this, pGV1428pflA, pGV1429pflB and pGV1002fdh1 are digested with EcoICRI+XhoI restriction enzymes and ligated into pGV1208KI, pGV1209KI and pGV1213KI digested with BamHI (and subsequently blunt ended with Klenow fill-in)+XhoI using standard molecular biology methods (Sambrook, J. Fritsch, E. F., Maniatis, T., 1989) to yield pGV1208KIPflA, pGV1209KIPflB and pGV1213KIFdh1. The resulting plasmids along with pGV1227KI are transformed into a strain of K. lactis (MATa, pdc1, trp1, his3, leu2 ura3)) and selected for His, Leu, Trp and Ura prototrophy. Kluyveromyces lactis transformants harboring pGV1428, pGV1429, pGV1430 and pGV1431 are used as control isolates Production of butanol is performed as described in Example 4. The expected n-butanol yield is greater than 10%.

### Example 31

### (Prophetic). Pyruvate Formate Lyase and Formate Dehydrogenase I Expression in Kluyveromyces lactis Lacking Pyruvate Decarboxylase Activity

Cloning of E. coli pflB (inactive Pyruvate formate lyase) and pflA (Pyruvate formate lyase activating enzyme) Cb-FDH1 are as described in Example 30.

The resulting plasmids (pGV1428pflA, pGV1429pflB and pGV1430fdh1) and vectors (pGV1428, pGV1429 and pGV1430) are utilized to transform yeast strain K. lactis (MatA, pdc1, trp1, his3, leu2 and ura3) by known methods (Kooistra R, Hooykaas P J, Steensma H Y. (2004) Yeast. 15; 21(9):781-92) to yield PflA, PflB, Cb-Fdh1 expressing (PFL+) and control (PFL−) transformants. Both sets of transformants are chosen by selection for HIS, TRP and LEU prototrophy.

The resulting trasformants are evaluated for PflA, PflB and Cb-Fdh1 expression using crude yeast protein extracts and western blot analysis as described in Example 2.

Those yeast transfromants verified to express all three proteins are assessed for cellular acetyl-CoA levels in comparison to the vector only control transformants. For this, PFL+ and PFL− cells are grown in SC-LEU, HIS, TRP medium in shake flask format and assessed as described in Example 30.

To evaluate how the expression of PflA, PflB and Fdh1 results in higher butanol production, pGV1208KIPflA, pGV1209KIPflB and pGV1213KIFdh1 along with pGV1227KI are transformed into K. lactis (MAT a, pdc1Δ, trp1, his3, leu2, ura3) and selected for His, Leu, Trp and Ura prototrophy. Kluyveromyces lactis transformants harboring pGV1428, pGV1429, pGV1430 and pGV1431 are used as control isolates. Production of butanol is performed as described in Example 4. The expected n-butanol yield is greater than 50%.

### Example 32

### (Prophetic). Pfl (Pyruvate Formate Lyase) and Fdh1 (Formate Dehydrogenase I) Expression in a Kluyveromyces lactis Devoid of Adh1 Activity

Cloning of E. coli pflB (inactive Pyruvate formate lyase), pflA (Pyruvate formate lyase activating enzyme) and Cb-FDH1 are described in Example 30.

The resulting plasmids (pGV1428pflA, pGV1429pflB and pGV1430fdh1) and vectors (pGV1428, pGV1429 and pGV1430) are utilized to transform yeast strain K. lactis (MAT a, trp1, his3, leu2, ura3) by known methods (Kooistra R, Hooykaas P J, Steensma H Y. (2004) Yeast. 15; 21(9):781-92) to yield PflA, PflB, Fdh1 expressing (EcPFL+) and control (EcPFL−) transformants. Both sets of transformants are chosen by selection for HIS, TRP and LEU prototrophy.

The resulting trasformants are evaluated for PflA, PflB and Fdh1 expression using crude yeast protein extracts and western blot analysis as described in Example 2.

Those yeast transfromants verified to express all three proteins are assessed for cellular acetyl-CoA levels in comparison to the vector only control transformants. For this, EcPFL+ and EcPFL− cells are grown in SC-LEU, HIS, TRP medium in shake flask format and assessed as described in Example 30.

To evaluate how the expression of PflA, PflB and Fdh1 results in higher butanol production, pGV1208KIPflA, pGV1209KIPflB and pGV1213KIFdh1 along with pGV1227KI are transformed into K. lactis (MAT a, adh1Δ, trp1, his3, leu2, ura3) and selected for His, Leu, Trp and Ura prototrophy. Kluyveromyces lactis transformants harboring pGV1428, pGV1429, pGV1430 and pGV1431 are used as control isolates. Production of butanol is performed as described in Example 4. The expected n-butanol yield is greater than 20%.

### Example 33

### (Prophetic). KIALD6 Overexpression in Kluyveromyces lactis

To clone KIALD6, genomic DNA of Kluyveromyces lactis is used as a template in a PCR reaction with primers KIALD6_left5 and KIALD6_right3 (see Table 1), which is otherwise assembled as described in Example 5. The aforementioned primers contain SalI and BamHI restriction sites, respectively, and the resulting PCR fragment is digested with SalI+BamHI and ligated into similarly restriction digested pGV1428 to yield pGV1428KIALD6. Subsequently, KIALD6 is subcloned by digestion of pGV1428ALD6 with EcoICRI+XhoI and ligation into BamHI (and subsequently blunt ended by Klenow fill-in)+XhoI-digested pGV1208KI to yield pGV1208KIALD6.

The resulting plasmid, pGV1428ALD6KI, and vector, pGV1428 are utilized to transform yeast strain K. lactis (MAT a, trp1, his3, leu2, ura3) by known methods (Kooistra R, Hooykaas P J, Steensma H Y. (2004) Yeast. 15; 21(9):781-92) to yield KIALD6+ and KIALD6− over-expressing and control transformants, respectively. Both sets of transformants are chosen by selection for HIS prototrophy.

The resulting trasformants, KIALD6+ and KIALD6− are evaluated for KIAld6 expression using crude yeast protein extracts and Western blot analysis as described in Example 2.

Those K. lactis transfromants verified to overexpress KIAld6 protein are assessed for enhanced acetaldehyde dehydrogenase activity in comparison to the vector-only control transformants. For this, KIALD6+ and KIALD6− cells are grown in SC-HIS medium in shake flask format and assessed as described in Example 23.

To evaluate how the overexpression of KIALD6 results in higher butanol production, pGV1208KIALD6 is transformed into K. lactis (MAT a, trp1, his3, leu2, ura3) along with pGV1209KI, pGV1227KI and pGV1213KI and selected for HIS, LEU, TRP and URA prototrophy Transformants arising from K. lactis transformed with pGV1428, pGV1429, pGV1430 and pGV1431 are used as control isolates. Production of butanol is performed as described in Example 4. The expected n-butanol yield is greater than 5%.

### Example 34

### (Prophetic). Overexpression of an Aldehyde Dehydrogenase in Kluyveromyces lactis Devoid of Adh1 Activity

Cloning of Kluyveromyces KIALD6 gene is described in Example 33.

The resulting plasmid, pGV1428ALD6, and vector, pGV1428 are utilized to transform yeast strain K. lactis (MATa, adh1Δ, trp1, his3, leu2, ura3) by known methods (Kooistra R, Hooykaas P J, Steensma H Y. (2004) Yeast. 15; 21(9):781-92) to yield KIALD6+ and KIALD6− over-expressing and control transformants, respectively. Both sets of transformants are chosen by selection for HIS prototrophy.

The resulting trasformants—are evaluated for KIAld6 expression using crude yeast protein extracts and Western blot analysis as described in Example 2.

Those K. lactis transfromants verified to express KIAld6 proteins are assessed for enhanced acetaldehyde dehydrogenase activity as described in Example 30.

To evaluate how overexpression of KIAld6 results in higher butanol production, pGV1208KIALD6 is transformed into K. lactis (MAT a, adh1Δ, trp1, his3, leu2, ura3) along with pGV1209KI, pGV1227KI and pGV1213KI and selected for HIS, LEU, TRP and URA prototrophy Transformants arising from K. lactis transformed with pGV1428, pGV1429, pGV1430 and pGV1431 are used as control isolates. Production of butanol is performed as described in Example 4. The expected n-butanol yield is greater than 10%.

### Example 35

### (Prophetic). Overexpression of an acetyl-CoA Synthase Gene in the Yeast Kluyveromyces lactis

Two paralagous genes, KIACS1 and KIACS2, encode acetyl-CoA activity in the genome of the yeast Kluyveromyces lactis. To clone KIACS1 and KIACS2, Kluyveromyces lactis genomic DNA is utilized as template with primers KIACS1_left5 & KIACS2_Right3 (ACS1) and KIACS2_Left5 & KIACS2_Right3 (ACS2) (see Table 1), containing NotI & SalI and SalI & BamHI restriction sites in the forward and reverse primers, respectively. The resulting PCR fragments are digested with appropriate enzymes and ligated into similarly restriction digested pGV1429 and pGV1431 to yield pGV1429ACS1 and pGV1431ACS2. Subsequently, KIACS1 and KIACS2 are subcloned by digestion of pGV1429ACS1 and pGV1431ACS2 with SacI & NotI and ligation into similarly digested pGV1209KI and pGV1213KI to yield pGV1209KIACS1 and pGVKIACS2.

The resulting plasmids, pGV1429ACS1 and pGV1431ACS2 and empty vectors pGV1429 and pGV1431 are utilized to transform K. lactis (MATa, trp1, his3, leu2, ura3) by known methods to yield KIACS1+, KIACS2+ and KIACS− protein over-expressing and control transformants, respectively. Both sets of transformants are chosen by selection for TRP, URA prototrophy. The trasformants are evaluated for KIAcs1 and KIAcs2 expression using crude yeast protein extracts and western blot analysis as described in Example 2.

Those yeast transfromants verified to express KIAcs1 and KIAcs2 proteins are assessed for enhanced acetyl-CoA synthase activity in comparison to the vector only control transformants. For this, KIACS1+, KIACS2+ and KIACS− cells are grown in SC-TRP, URA medium in shake flask format and assessed as described in Example 25.

To evaluate how the overexpression of KIACS1 and KIACS2 result in higher butanol production, pGV1209KIACS1 and pGV1209KIACS2 are transformed into strain Gevo 1287 along with pGV1208KI and pGV1227KI, and transformed cells are selected for His, Leu, Trp and Ura prototrophy. Transformants resulting from a K. lactis (MAT a, trp1, his3, leu2, ura3) transformed with pGV1428, pGV1429, pGV1430 and pGV1431 are used as control isolates. Production of butanol is performed as described in Example 4. The expected n-butanol yield is greater than 5%.

### Example 36

### (Prophetic). Overexpression of an acetyl-CoA Synthase Gene in a Yeast Kluyveromyces lactis Devoid of Adh1 Activity

Cloning of KIACS1 and KIACS2 genes of Kluyveromyces lactis is described in Example 35.

The resulting plasmids, pGV1429ACS1 and pGV1431ACS2 and empty vectors pGV1429 and pGV1431 are utilized to transform K. lactis (MATa, adh1Δ, trp1, h is 3, leu2, ura3) by known methods to yield KIACS1+ and KIACS2+ overexpressing and control transformants, respectively. Both sets of transformants are chosen by selection for TRP and URA prototrophy. The trasformants are evaluated for KIAcs1 and KIAcs2 expression using crude yeast protein extracts and Western blot analysis as described in Example 2.

Those yeast transfromants verified to express KIAcs1 and KIAcs2 proteins are assessed for enhanced acetyl-CoA synthase activity as described in Example 25.

To evaluate how the over-expression of KIACS1 and KIACS2 result in higher butanol production, pGV1209KIACS1 and pGV1209KIACS2 are transformed into K. lactis (MatA, adh1, trp1, his3, leu2 and ura3) along with pGV1208KI and pGV1227KI. Production of butanol is performed as described in Example 4. The expected n-butanol yield is greater than 10%.

### Example 37

### (Prophetic). KIALD6 and KIACS1 or KIACS2 Over-Expression in Kluyveromyces lactis

KIALD6, KIACS1 and KIACS2 genes are cloned as described above in Examples 33 and 35.

The resulting plasmids pGV1428ALD6 and pGV1429ACS1 or pGV1430ACS2 and vectors pGV1428 and pGV1429 or pGV1430 are utilized to transform K. lactis (MATa, trp1, his3, leu2, ura3) by known methods to yield KIALD6+KIACS1+, KIALD6+KIACS2+ and KIALD-KIACS−, over-expressing and control transformants, respectively. Both sets of transformants are chosen by selection for HIS, TRP and HIS, LEU prototrophy, respectively.

Transformants KIALD6+KIACS1+ and KIALD6+KIACS2+ are assessed for enhanced Acetyl-CoA synthase activity in comparison to the vector only control transformants (ALD-ACS−). For this, KIALD6+KIACS1+, KIALD6+KIACS2+ and KIALD-KIACS− cells are grown in SC-HIS, TRP and HIS, LEU media, respectively, in shake flask format and assessed as described in Example 25.

To evaluate how the overexpression of KIAld6 and KIAcs1 or KIAcs2 result in higher butanol production, K. lactis (MATa, trp1, his3, leu2 ura3) is transformed with pGV1208KIALD6, pGV1209KIACS1 or pGV1209KIACS2, pGV1227KI, pGV1213KI and selected for HIS, LEU, TRP and URA prototrophy. Transformants resulting from K. lactis (MATa, trp1, his3, leu2 ura3) transformed with pGV1428, pGV1429, pGV1430 and pGV1431 are used as control isolates. Production of butanol is performed as described in Example 4. The expected n-butanol yield is greater than 5%.

### Example 38

### (Prophetic). KIALD6, KIACS1 and KIACS2 Over-Expression in Kluyveromyces lactis Devoid of KIAdh1 Activity (KIadh1Δ)

KIALD6, KIACS1 and KIACS2 genes are cloned as described in Examples 33 and 35.

The resulting plasmids pGV1428ALD6 and pGV1429ACS1 or pGV1430ACS2 and vectors pGV1428 and pGV1429 or pGV1430 are utilized to transform K. lactis (MATa, KIadh1Δtrp1, his3, leu2 ura3) by known methods to yield KIALD6+KIACS1+, KIALD6+KIACS2+ and KIALD-KIACS−, over-expressing and control transformants, respectively. Both sets of transformants are chosen by selection for HIS, TRP and HIS, LEU prototrophy, respectively.

Transformants, KIALD6+KIACS1+ and KIALD6+KIACS2+ are assessed for cellular acetyl CoA levels as described in Example 14.

To evaluate whether the over-expression of KIAld6 and KIAcs1 or KIAcs2 result in higher butanol production, K. lactis (MATa, KIadh1Δtrp1, his3, leu2 ura3) is transformed with pGV1208KIALD6, pGV1209KIACS1 or pGV1209KIACS2, pGV1227KI, pGV1213KI. Production of butanol is performed as described in Example 4. The expected n-butanol yield is greater than 10%.

What is claimed is:

1. A metabolically-engineered yeast capable of metabolizing a carbon source to produce n-butanol, at least one pathway configured for producing an increased amount of cytosolic acetyl-CoA relative to another amount of cytosolic acetyl-CoA produced by a wild-type yeast, and at least one heterologous gene to encode and express at least one enzyme for a metabolic pathway capable of utilizing NADH to convert acetyl-CoA to the n-butanol.

2. The yeast of claim 1, wherein the at least one heterologous gene alone encodes and expresses the at least one enzyme for the metabolic pathway capable of utilizing NADH to convert acetyl-CoA to the n-butanol.

3. The yeast of claim 1, wherein the at least one heterologous gene in combination with at least one native yeast gene encodes and expresses the at least one enzyme for the metabolic pathway capable of utilizing NADH to convert acetyl-CoA to the n-butanol.

4. The yeast of claim 1, wherein the yeast overexpresses a pyruvate decarboxylase to increase the production of cytosolic acetyl-CoA.

5. The yeast of claim 4, wherein the pyruvate decarboxylase is encoded by S. cerevisiae gene PDC1.

6. The yeast of claim 4, wherein the pyruvate decarboxylase is encoded by at least one of S. cerevisiae gene PDC1, PDC5, and PDC6.

7. The yeast of claim 1, wherein the yeast overexpresses an aldehyde dehydrogenase to increase production of cytosolic acetyl-CoA.

8. The yeast of claim 7, wherein the aldehyde dehydrogenase is encoded by S. cerevisiae gene ALD6.

9. The yeast of claim 7, wherein the aldehyde dehydrogenase is encoded by K. lactis gene ALD6.

10. The yeast of claim 1, wherein the yeast overexpresses acetyl-CoA synthetase to increase production of cytosolic acetyl-CoA.

11. The yeast of claim 10, wherein the acetyl-CoA synthetase is encoded by at least one of S. cerevisiae gene ACS1 and S. cerevisiae gene ACS2.

12. The yeast of claim 10, wherein the acetyl-CoA synthetase is encoded by at least one of K. lactis gene ACS1 and K. lactis gene ACS2.

13. The yeast of claim 1, wherein the yeast overexpresses both aldehyde dehydrogenase and acetyl-CoA synthetase to increase production of cytosolic acetyl-CoA.

14. The yeast of claim 13, wherein the aldehyde dehydrogenase is encoded by S. cerevisiae gene ALD6, and the acetyl-CoA synthetase is encoded by at least one of S. cerevisiae gene ACS1 and S. cerevisiae gene ACS2.

15. The yeast of claim 13, wherein the aldehyde dehydrogenase is encoded by K. lactis gene ALD6, and the acetyl-CoA synthetase is encoded by at least one of K. lactis gene ACS1 and K. lactis gene ACS2.

16. The yeast of claim 13, wherein the yeast overexpresses a pyruvate decarboxylase to increase production of cytosolic acetyl-CoA.

17. The yeast of claim 16, wherein the pyruvate decarboxylase is encoded by at least one of PDC1, PDC5 and PDC6, aldehyde dehydrogenase is encoded by S. cerevisiae gene ALD6, and the acetyl-CoA synthetase is encoded by at least one of S. cerevisiae gene ACS1 and S. cerevisiae gene ACS2.

18. The yeast of claim 16, wherein the pyruvate decarboxylase is encoded by K. lactis PDC1, aldehyde dehydrogenase is encoded by K. lactis gene ALD6, and the acetyl-CoA synthetase is encoded by at least one of K. lactis gene ACS1 and K. lactis gene ACS2.

19. The yeast of claim 1, wherein the yeast overexpresses a pyruvate dehydrogenase to increase production of cytosolic acetyl-CoA.

20. The yeast of claim 19, wherein the yeast overexpresses a pyruvate dehydrogenase encoded by E. coli genes aceE, aceF, lpdA so as to increase production of cytosolic acetyl-CoA.

21. The yeast of claim 20, wherein PDC activity is one of reduced and eliminated.

22. The yeast of claim 19, wherein the yeast overexpresses a pyruvate dehydrogenase encoded by N-terminal mitochondrial targeting signal deleted S. cerevisiae genes PDA1, PDB1, PDX1, LAT1, LPD1 so as to increase production of cytosolic acetyl-CoA.

23. The yeast of claim 22, wherein PDC activity is one of reduced and eliminated.

24. The yeast of claim 23, wherein the yeast is S. cerevisiae of one of (1) genotype pdc2Δ, and (2) genotype pdc1Δ, genotype pdc5Δ, and genotype pdc6Δ.

25. The yeast of claim 23, wherein the yeast is K. lactis of genotype pdc1Δ.

26. The yeast of claim 1, wherein the yeast overexpresses both a pyruvate formate lyase and a formate dehydrogenase to increase the production of cytosolic acetyl-CoA.

27. The yeast of claim 26, wherein the yeast overexpresses a pyruvate formate lyase encoded by E. coli gene pflA and E. coli gene pflB, and in combination with C. boidini gene FDH1 so as to increase production of cytosolic acetyl-CoA.

28. The yeast of claim 27, wherein PDC activity is one of reduced and eliminated.

29. The yeast of claim 27, where the yeast is S. cerevisiae of one of (1) genotype pdc2Δ, and (2) genotype pdc1Δ, genotype pdc5Δ, and genotype pdc6Δ.

30. The yeast of claim 27, where the yeast is K. lactis of the genotype pdc1Δ.

31. The yeast of claim 1, wherein at least one of the at least one heterologous gene has been subjected to molecular evolution to enhance the enzymatic activity of the protein encoded thereby.

32. The yeast of claim 1, wherein at least one additional gene encoding alcohol dehydrogenase is inactivated so that alcohol dehydrogenase activity is reduced sufficiently to increase cytosolic acetyl-CoA production relative to wild-type production.

33. The yeast of claim 32, wherein the yeast is S. cerevisiae, and the alcohol dehydrogenase is encoded by ADH1.

34. The yeast of claim 32, wherein the yeast is K. lactis, and the alcohol dehydrogenase is encoded by ADH1.

35. The yeast of claim 32, wherein the yeast is S. cerevisiae, and the alcohol dehydrogenase is encoded by ADH1, ADH2, ADH3 and ADH4.

36. The yeast of claim 32, wherein the yeast is K. lactis, and the alcohol dehydrogenase is encoded by ADHI, ADHII, ADHIII and ADHIV.

37. The yeast of claim 1, wherein the yeast is a species from a genus of one of Saccharomyces, Dekkera, Pichia, Hansenula, Yarrowia, Aspergillus, Kluyveromyces, Pachysolen, Schizosaccharomyces, Candida, Trichosporon, Yamadazyma, Torulaspora, and Cryptococcus.

38. The yeast of claim 1, wherein the pathway provides for balanced NADH production and consumption when metabolizing the carbon source to produce n-butanol.

39. A method of producing n-butanol, the method comprising:
(a) providing metabolically-engineered yeast capable of metabolizing a carbon source to produce n-butanol, at least one pathway configured for producing an increased amount of cytosolic acetyl-CoA relative to another amount of cytosolic acetyl-CoA produced by a wild-type yeast, and at least one heterologous gene to encode and express at least one enzyme for a metabolic pathway capable of utilizing NADH to convert acetyl-CoA to the n-butanol; and
(b) culturing the metabolically-engineered yeast for a period of time and under conditions to produce the n-butanol.

40. A method of producing n-butanol, using yeast, the method comprising:
(a) metabolically engineering the yeast to increase cytosolic acetyl-CoA production;
(b) metabolically engineering the yeast to express a metabolic pathway that converts a carbon source to n-butanol, wherein the pathway requires at least one non-native enzyme of the yeast, wherein steps (a) and (b) can be performed in either order; and
(c) culturing the yeast for a period of time and under conditions to produce a recoverable amount of n-butanol.

41. A method of producing n-butanol, using yeast, the method comprising:
(a) culturing a metabolically-engineered yeast for a period of time and under conditions to produce a yeast-cell biomass without activating n-butanol production; and
(b) altering the culture conditions for another period of time and under conditions to produce a recoverable amount of n-butanol.

42. A metabolically-engineered yeast capable of metabolizing a carbon source and producing an increased amount of acetyl-CoA relative to the amount of cytosolic acetyl-CoA produced by a wild-type yeast.

43. The yeast of claim 42, wherein the yeast overexpresses a pyruvate decarboxylase, aldehyde dehydrogenase and acetyl-CoA synthetase to increase the production of cytosolic acetyl-CoA.

44. The yeast of claim 42, wherein the pyruvate decarboxylase is encoded by at least one of S. cerevisiae gene PDC1, PDC5 and PDC6 aldehyde dehydrogenase is encoded by S. cerevisiae ALD6 and acetyl-CoA synthetase is endcoded by at least one of S. cerevisiae genes ACS1 and ACS2.

45. The yeast of claim 44, wherein the alcohol dehydrogenase is inactivated by the deletion of S. cerevisiae gene ADH1.

46. The yeast of claim 42, wherein the yeast is of the genus Kluyveromyces, the pyruvate decarboxylase is encoded by K. lactis gene KIPDC1, aldehyde dehydrogenase is encoded by K. lactis gene KIALD6 and acetyl-CoA synthetase is encoded by at least one of K. lactis genes KIACS1 and KIACS2.

47. The yeast of claim 46, wherein the alcohol dehydrogenase is inactivated by the deletion of K. lactis gene ADH1.

48. The yeast of claim 42, wherein the yeast overexpresses a pyruvate dehydrogenase to increase production of cytosolic acetyl-CoA.

49. The yeast of claim 48, wherein the yeast overexpresses a pyruvate dehydrogenase encoded by E. coli gene aceE, E. coli gene aceF and E. coli gene lpdA so as to increase production of cytosolic acetyl-CoA.

50. The yeast of claim 49, wherein PDC activity is one of reduced and eliminated.

51. The yeast of claim 49, where the yeast is S. cerevisiae of one of (1) genotype pdc2Δ, and (2) genotype pdc1Δ, genotype pdc5Δ, and genotype pdc6Δ.

52. The yeast of claim 49, where the yeast is K. lactis of the genotype pdc1Δ.

53. The yeast of claim 48, wherein the yeast overexpresses a pyruvate dehydrogenase encoded by N-terminal mitochondrial targeting signal deleted S. cerevisiae genes PDA1, PDB1, PDX1, LAT1, and LPD1 so as to increase production of cytosolic acetyl-CoA.

54. The yeast of claim 53, wherein PDC activity is one of reduced and eliminated.

55. The yeast of claim 53, where the yeast is S. cerevisiae of one of (1) genotype pdc2Δ, and (2) genotype pdc1Δ, genotype pdc5Δ, and genotype pdc6Δ.

56. The yeast of claim 53, where the yeast is K. lactis of the genotype pdc1Δ.

57. The yeast of claim 42, wherein the yeast overexpresses both a pyruvate formate lyase and a formate dehydrogenase so as to increase the production of cytosolic acetyl-CoA.

58. The yeast of claim 57, wherein the yeast overexpresses a pyruvate formate lyase encoded by E. coli genes pflA, pflB, and in combination with C. boidini gene FDH1 so as to increase production of cytosolic acetyl-CoA.

59. The yeast of claim 58, wherein PDC activity is one of reduced and eliminated.

60. The yeast of claim 59, wherein the yeast is S. cerevisiae of one of (1) genotype pdc2Δ, and (2) genotype pdc1Δ, genotype pdc5Δ, and genotype pdc6Δ.

61. The yeast of claim 59, wherein the yeast is K. lactis of genotype pdc1.

62. The yeast of claim 42, wherein at least one of gene have been subjected to molecular evolution so as to enhance enzymatic activity of a protein encoded thereby.

63. A method of increasing metabolic activity of yeast, the method comprising producing an increased amount of cytosolic acetyl-CoA of the yeast relative to another amount of cytosolic acetyl-CoA produced by a wild-type yeast.

64. A metabolically-engineered yeast having at least one pathway configured for producing an increased amount of cytosolic acetyl-CoA relative to another amount of cytosolic acetyl-CoA produced by a wild-type yeast.


SEQUENCE LISTING (190 sequences)

SEQ ID NO: 1 - DNA, 1179 bp, Artificial Sequence
  Description of Artificial Sequence Synthetic Ca-thl-co polynucleotide
atgaaagaagttgtaatagctagcgcggtgcgtaccgccattggctcttatggtaaaagtctgaaggatgttccggcagtcgacttaggggctacggcgatcaaagaagccgtaaaaaaggcaggaattaaaccagaggatgtgaatgaagttatcctgggcaacgtcctgcaggctggtttagggcaaaatcctgcgcgccaggcctcatttaaagcaggactgccggtagagattccagctatgactatcaacaaggtgtgcggctccggtctgcggacagtttcgttagcggcccaaattatcaaagcaggcgacgctgatgtcattatcgcgggtgggatggaaaatatgagccgtgccccttacctggcaaacaatgcgcgctggggatatcgtatgggcaacgctaaattcgtggacgaaatgattaccgatggtctgtgggatgcctttaatgactaccatatgggcatcacggcagagaacattgcggaacgctggaatatctctcgggaggaacaggatgagttcgctttagccagtcagaagaaagcagaggaagcgattaaatcaggtcaatttaaggacgagatcgtaccggttgtgattaaagggcgtaaaggagaaactgtcgttgatacagacgaacacccgcgcttcggctccaccattgagggtctggctaagctgaaaccagcctttaaaaaggatgggacggtaaccgcaggcaacgcgtcgggtttaaatgattgtgccgcagtgctggtcatcatgagcgcggaaaaagctaaagagctgggagttaagcctctggccaaaattgtgtcttatggcagtgcgggtgtagacccggctatcatggggtacggcccgttctatgcaactaaagccgcgattgaaaaggctggttggacagtcgatgaattagacctgatcgagtcaaacgaagcatttgccgcgcagtccctggctgttgcaaaagatttaaaattcgatatgaataaggtgaacgtaaatggaggcgccattgcgctgggtcatccaatcggggcttcgggagcacgtattctggttacgttagtgcacgccatgcaaaaacgcgacgcgaaaaagggcctggctaccctgtgcatcggtgggggccagggtactgcaatattgctagaaaagtgctag

SEQ ID NO: 2 - DNA, 849 bp, Artificial Sequence
  Description of Artificial Sequence Synthetic Ca-hbd-co polynucleotide
atgaaaaaggtatgtgttataggcgcgggaaccatgggtagcggtattgcccaggcatttgctgcaaaaggtttcgaagtggttctgcgtgatatcaaggacgagtttgtcgatcgcggcttagacttcattaataaaaacctgtctaaactggtaaagaaagggaaaatcgaagaggcgacgaaggtggaaattttaactcggatcagtggaacagttgatctgaatatggccgctgactgcgatctggtcattgaagcggccgtagagcgtatggatatcaaaaaacaaatttttgcagacttagataacatctgtaagccggaaaccattctggcttcaaatacgtcctcgctgagcatcactgaggtggcgtctgccacaaaacgcccagacaaagttattggcatgcatttctttaaccctgcaccggtcatgaagttagtggaagtaatccgtgggattgctaccagtcaggaaacgttcgatgcggttaaagagacctcaatcgccattggaaaagacccagtggaagtcgcagaggcgcctggctttgttgtaaatcgcattctgatcccgatgattaacgaagctgtgggaatcctggccgaaggaattgcatccgtcgaggatatcgacaaggcgatgaaattaggcgctaatcacccgatgggtccactggaactgggcgacttcattggtctggatatctgcttagccattatggacgttctgtattcggagactggggatagcaaataccggcctcatacactgttaaagaaatatgtgcgtgcaggatggctgggccgcaaatctggtaagggtttctacgattattcaaaataa

SEQ ID NO: 3 - DNA, 786 bp, Artificial Sequence
  Description of Artificial Sequence Synthetic Ca-crt-co polynucleotide
atggaactaaacaatgtcatcctggaaaaagagggcaaggtggcggttgtcaccattaatcgtccgaaagccttaaacgcactgaatagcgatacgctgaaagaaatggactatgtaatcggtgagattgaaaacgattctgaagtgttagctgttatcctgactggggcgggagagaagagttttgtcgccggcgcagacatttcagaaatgaaagagatgaatacaatcgaaggtcgcaaattcgggattctgggaaacaaggtatttcggcgtttagaactgctggagaaaccagtgatcgctgcggttaatggcttcgccttaggtggcggttgcgaaattgcaatgtcctgtgatatccgcattgcttcgagcaacgcgcgttttgggcagcctgaggtcggactgggcatcacaccgggtttcggcggtacgcaacgcctgtctcggttagtggggatgggaatggccaaacagctgatttttactgcacaaaatatcaaggctgacgaagcgctgcgtattggcctggtaaacaaagttgtggaaccaagtgagttaatgaatacagccaaagaaatcgcaaacaagattgtctcaaatgcgcctgttgctgtaaaactgtccaaacaggccattaaccgcggtatgcagtgcgatatcgacaccgcactggcgttcgagtcggaagcttttggggaatgtttcagcacggaggaccaaaaggatgccatgaccgcatttattgaaaaacgtaaaattgaaggcttcaaaaatagatag

SEQ ID NO: 4 - DNA, 1140 bp, Artificial Sequence
  Description of Artificial Sequence Synthetic Ca-bcd-co polynucleotide
atggattttaatttaacaagagaacaggaactggtccgtcagatggtacgtgaatttgcagaaaacgaggttaaaccgattgctgcagagattgatgagactgaacgcttcccgatggaaaacgtcaaaaagatgggtcagtatggcatgatgggcattccgttctctaaagagtacggcggtgcgggtggcgacgttctgtcttatatcatcgctgtagaggaactgtccaaagtatgtggcaccacgggcgtgatcctgtccgcgcacacctctctgtgcgcaagcctgatcaacgaacacggcaccgaggaacagaagcaaaaatacctggtcccgctggccaaaggtgaaaagatcggtgcatacggtctgacggaaccgaacgcaggtacggacagcggcgcacaacagacggttgcggtactggaaggcgaccactacgttattaacggtagcaaaatcttcatcacgaacggtggcgtggctgacacctttgttatcttcgcgatgaccgaccgcactaaaggcactaaaggtatctctgcgttcatcatcgagaagggtttcaagggtttttctatcggcaaagtggaacagaagctgggtatccgtgcctcctctactaccgagctggttttcgaagacatgattgtgccggttgaaaatatgatcggcaaagaaggcaaaggcttcccgatcgctatgaaaaccctggatggcggccgtatcggcattgcagcacaggcactgggtatcgcagaaggcgctttcaacgaagcacgtgcgtacatgaaagaacgtaaacagtttggccgttctctggataaatttcaaggcctggcgtggatgatggcagacatggacgtagcgattgaatctgcgcgctacctggtctataaagcagcttacctgaaacaggcaggtctgccttacaccgttgacgcagcacgtgcgaaactgcacgcggccaacgttgccatggatgttaccaccaaagccgtgcaactgtttggcggttacggctatactaaggattatccggttgaacgtatgatgcgtgacgcgaaaatcaccgaaatctatgaaggtacttccgaagtgcagaaactggtcatttcaggaaaaatttttagttaa

SEQ ID NO: 5 - DNA, 1011 bp, Artificial Sequence
  Description of Artificial Sequence Synthetic Ca-eftA-co polynucleotide
atgaataaagcagattacaagggcgtttgggtctttgcggaacagcgtgatggtgaactgcagaaagtgtccctggaactgctgggcaaaggcaaggagatggcagaaaaactgggtgttgaactgaccgcagttctgctgggtcacaacactgaaaagatgtccaaagacctgctgtcccatggcgcagacaaggtgctggctgcggacaacgaactgctggctcactttagcaccgacggttatgcaaaagtaatctgcgacctggttaacgagcgcaagccggaaatcctgttcatcggcgccacttttattggtcgcgacctgggccctcgtattgctgcgcgtctgtccactggcctgactgcggattgcacctccctggacattgatgttgaaaaccgtgatctgctggcaactcgcccggcattcggcggcaacctgatcgccaccatcgtatgttccgaccaccgtccgcaaatggctactgtacgtccgggcgtatttgaaaagctgccggtgaacgacgcaaacgtttccgacgacaaaatcgaaaaagttgctatcaagctgaccgctagcgatatccgtaccaaagtttctaaagtagtgaaactggcgaaggacatcgcagatattggtgaagcaaaagttctggtggcaggcggtcgtggcgtcggttccaaagagaacttcgaaaaactggaggaactggcgtctctgctgggcggtactattgcagcgtcccgtgcagcaatcgaaaaagaatgggtggacaaggatctgcaggtgggccagactggtaaaaccgttcgtccgaccctgtacatcgcctgcggcatctccggtgctattcagcacctggccggcatgcaggacagcgactacatcatcgccatcaacaaagacgttgaagctccgatcatgaaagtggcggacctggcaatcgttggtgacgtgaacaaagttgttccggaactgatcgcgcaggttaaagctgctaataattaa

SEQ ID NO: 6 - DNA, 780 bp, Artificial Sequence
  Description of Artificial Sequence Synthetic Ca-eftB-co polynucleotide
atgaatatagttgtttgtttaaaacaggtcccggacaccgcagaagttcgtattgatccagtaaagggtacgctgattcgcgagggcgtgccgtctatcatcaacccagatgacaagaacgccctggaagaagcactggtcctgaaagataattacggcgctcacgtaactgttatctctatgggtccgccgcaagcgaaaaatgcgctggttgaagctctggcgatgggcgctgacgaggctgttctgctgactgatcgtgctttcggtggtgcggacaccctggccacttcccacactatcgcggcaggtatcaagaaactgaaatatgacattgtgtttgctggtcgtcaggctattgacggtgacacggcacaggtaggcccggaaatcgccgaacacctgggtattccgcaggtgacctacgtagaaaaagtagaagtagacggtgataccctgaaaatccgcaaagcatgggaagatggctacgaggtggttgaagtaaaaaccccggtactgctgaccgctatcaaagagctgaatgtaccgcgttacatgtctgttgagaaaatcttcggcgcgttcgacaaggaagtaaagatgtggaccgctgatgatattgacgttgacaaagcgaatctgggcctgaagggctccccaactaaagttaagaagtcctctactaaagaagtgaagggtcagggtgaggtgattgataaacctgttaaagaagctgctgcgtacgtggtttctaagctgaaagaagaacactatatttaa

SEQ ID NO: 7 - DNA, 2577 bp, Artificial Sequence
  Description of Artificial Sequence Synthetic Ca-adhE2-co polynucleotide
atgaaagttacaaatcaaaaagaactgaaacagaagttaaatgagctgcgtgaggcgcaaaaaaaatttgccacctatacgcaggaacaagtggataagattttcaaacagtgcgcaatcgctgcggccaaagaacgcattaacctggcaaagttagctgttgaagagactggcatcggtctggtcgaggacaaaattatcaaaaatcattttgcggccgagtacatttataacaagtacaaaaacgagaaaacctgtgggatcattgaccacgatgatagcctgggaatcacaaaggtagcagaaccgattggcatcgtggctgcgattgttccaacgactaatcctacatctaccgccatcttcaaaagtttaatttcactgaaaacgcggaatgcaatctttttctccccgcatccacgtgctaagaaatcgaccattgcggccgcaaaactgattttagacgcggctgtcaaggccggtgcacctaaaaacatcattgggtggatcgacgaaccgagcattgaactgtctcaggatctgatgagtgaggcggacatcattttagctactggaggcccgtcaatggtaaaagccgcatattcctcgggtaagccagcgatcggcgtgggtgctgggaatactcctgccattatcgacgaaagcgcagacattgatatggcggtttctagtatcattctgtcaaaaacgtacgacaacggagtcatctgcgcctccgaacagtcgattctggtgatgaatagcatctatgagaaagtaaaggaagagtttgttaaacgcggctcttacattctgaaccagaatgaaattgcaaaaatcaaggaaaccatgttcaaaaacggtgcgattaatgctgatatcgtgggcaaaagtgcctatattatcgcgaagatggctggtattgaggtcccgcaaactacaaaaatcttaattggggaagttcagtcagtagaaaaatccgagctgtttagccacgaaaagctgtcgccggtgttagcaatgtataaagtcaaagatttcgacgaggccctgaagaaagcgcagcgtctgatcgaattaggaggctctggtcataccagttcactgtacattgatagccaaaacaataaagacaaggttaaagaatttgggctggctatgaaaacgtcccgcacctttatcaacatgccatcgtctcagggcgcaagtggtgatttatataatttcgccattgcgcctagctttactctgggatgtggcacatggggtgggaactcagtgtcccaaaatgtagagccgaagcatctgctgaacatcaaatcggtcgctgaacggcgtgagaatatgttatggttcaaagttccacagaagatttactttaaatatggctgcctgcgcttcgcactgaaagaattaaaggatatgaacaaaaaacgtgcctttatcgtgacggacaaggatctgttcaaactgggttacgtaaataaaattaccaaggttttagacgaaattgatatcaaatattctatttttactgacatcaaaagcgatccgacaattgatagtgtgaagaaaggagcgaaagagatgctgaacttcgaacctgacacgatcatttcaatcggcggtgggtccccgatggatgctgcaaaggtcatgcatctgttatacgagtatccagaagccgaaattgagaatctggcgatcaactttatggacattcgcaaacggatctgtaattttccgaaactgggaaccaaggctattagcgttgcaatccctactacggccggcaccggttcggaagcgacaccgttcgctgtgattaccaacgatgagactgggatgaaatatccactgacatcttacgaattaacgccgaatatggcaatcattgataccgaactgatgctgaacatgcctcgtaaattaactgccgcgacgggcattgacgcactggtacacgccatcgaggcgtatgtcagtgttatggcaaccgattacacagacgaactggcgttacgcgctattaagatgatctttaaatatctgccacgtgcctacaaaaatggtactaacgatattgaagcgcgcgagaagatggctcatgcatcaaatatcgccggaatggcgttcgctaacgcatttctgggcgtgtgccacagcatggcccataaattaggtgcgatgcaccatgtaccgcatgggattgcttgtgcagtcctgatcgaagaggttattaaatataatgccacggactgccctaccaagcagacagcgttcccgcaatacaaatccccaaacgctaaacggaagtatgcagaaatcgccgaatatctgaatctgaaaggcacttcggatacggagaaagtgaccgcgttaattgaagctatctctaagctgaaaattgatctgagtatcccgcagaacatttcagcagccggtattaataaaaaggacttttacaacaccttagataaaatgagcgagctggcgttcgacgatcaatgtacaactgctaatcctcgttatccgctgatctccgaattaaaagatatctatataaaatcattttaa

SEQ ID NO: 8 - DNA, 1152 bp, Artificial Sequence
  Description of Artificial Sequence Synthetic Me-bcd-co polynucleotide
atggattttaacttaacagatattcagcaagacttcctgaagctggcacacgactttggtgaaaagaaactggcccctactgttaccgaacgcgaccacaaaggtatctacgataaagaactgattgacgaactgctgtctctgggtatcaccggcgcatacttcgaagaaaaatacggcggtagcggtgacgacggtggcgatgtactgtcttatatcctggccgtagaagaactggcgaaatacgacgctggtgttgctatcactctgtctgccaccgtaagcctgtgtgcgaatccgatttggcagtttggtactgaggctcagaaagaaaagtttctggttccactggtcgaaggtactaaactgggtgcgtttggtctgaccgaaccgaacgcgggcactgatgcgagcggccagcaaactattgctactaaaaacgatgacggcacgtacaccctgaacggtagcaaaatcttcatcaccaacggtggcgctgccgatatctacatcgtatttgcgatgaccgacaaaagcaagggtaaccatggcatcaccgcgttcatcctggaagatggcactccgggtttcacctacggcaaaaaggaagataaaatgggtatccacacctctcagactatggaactggttttccaggacgttaaggtcccggccgagaacatgctgggcgaagaaggcaaaggcttcaagattgcaatgatgaccctggacggcggtcgcattggcgttgcggcccaggcactgggcatcgcagaggcagcgctggccgacgctgttgaatacagcaaacagcgtgttcagtttggcaaacctctgtgcaaattccaatccattagctttaagctggccgatatgaaaatgcagatcgaagccgcacgcaacctggtatataaagctgcatgcaagaaacaagaaggtaaaccgttcaccgtagacgctgcgatcgcgaaacgtgtagccagcgatgtggcaatgcgcgtgactaccgaagcagttcagattttcggtggctatggttactctgaagaatacccggtggctcgccacatgcgcgacgcaaaaatcactcagatctacgagggtacgaacgaagtgcagctgatggtcaccggcggtgctctgttaagttaa

SEQ ID NO: 9 - DNA, 1017 bp, Artificial Sequence
  Description of Artificial Sequence Synthetic Me-eftA-co polynucleotide
atggatttagcagaatacaaaggcatctacgtgatcgcagagcagttcgaaggtaaactgcgtgacgtttctttcgaactgctgggtcaagcgcgcatcctggcggacacgatcggcgacgaagtaggcgcaatcctgattggcaaagatgtaaaaccactggcgcaggaactgatcgcgcatggtgctcataaagtgtacgtctatgacgacccgcagctggaacattacaacacgactgcctatgccaaagtgatttgcgacttctttcatgaagagaaaccaaacgttttcctggttggtgcaactaacatcggtcgtgacctgggtccacgtgtagcgaacagcctgaaaaccggtctgactgcggattgtacccagctgggtgttgatgatgataagaaaaccatcgtttggacccgtccggcactgggcggcaacatcatggcggaaattatctgtccagataaccgcccgcagatgggcactgtgcgtcctcatgtcttcaaaaagccggaagccgacccgagcgcaactggtgaagtcattgaaaagaaagcgaacctgtctgacgctgatttcatgactaagttcgtagaactgatcaaactgggtggtgaaggcgttaaaatcgaggatgccgatgttattgttgctggtggccgtggcatgaatagcgaagagccttttaaaaccggtatcctgaaagagtgcgcggacgtactgggcggtgctgtcggtgccagccgtgccgccgtggacgcgggctggatcgacgctctgcaccaggtcggccagactggcaaaaccgttggtccgaaaatctacattgcttgtgcgattagcggtgctatccagccgctggcaggcatgacgggctctgattgtattatcgcaattaacaaagatgaagacgcgcctattttcaaggtgtgcgactatggcattgtgggcgatgtgttcaaagtgctgccactgctgactgaggcgatcaagaaacagaaaggcattgcataa

SEQ ID NO: 10 - DNA, 813 bp, Artificial Sequence
  Description of Artificial Sequence Synthetic Me-eftB-co polynucleotide
atggaaatattggtatgtgtcaaacaagtgccggatactgcagaagtcaaaattgatccggttaaacacaccgtgattcgtgcgggtgtgccgaatatcttcaacccgttcgaccaaaacgcgctggaagcggcgctggcgctgaaggacgcggataaagacgttaagattactctgctgtctatgggcccggaccaggcaaaagatgttctgcgtgaaggcctggccatgggcgctgatgacgcgtacctgctgtccgatcgtaaactgggtggctccgacactctggccaccggttatgctctggcccaggctattaagaaactggctgcggacaagggtattgagcaattcgacatcatcctgtgtggtaagcaagcgattgacggtgataccgctcaggtaggtccacagatcgcttgtgagctgggcatcccgcagatcacttatgctcgtgacatcaaggttgagggcgataaggttactgtgcagcaggaaaacgaagagggttacatcgtgaccgaagcgcagttcccggttctgatcaccgcggttaaagacctgaacgaacctcgtttcccgaccatccgtggcaccatgaaggcgaagcgtcgtgaaatcccgaacctggacgcagctgcagttgccgcggacgacgcgcagatcggcctgtccggttctccgaccaaagtacgcaaaattttcaccccaccgcagcgttccggcggtctggtactgaaagtggaagacgacaacgaacaggccattgtcgaccaggttatggaaaaactggttgcccagaaaatcatttaa

SEQ ID NO: 11 - DNA, 5024 bp, Artificial Sequence
  Description of Artificial Sequence Synthetic pGV1090 polynucleotide
ctagtgcttggattctcaccaataaaaaacgcccggcggcaaccgagcgttctgaacaaatccagatggagttctgaggtcattactggatctatcaacaggagtccaagcgagctcgatatcaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaactccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccagatatcgacgtctaagaaaccattattatcatgacattaacctataaaaataggcgtatcacgaggccctttcgtcttcacctcgagaaatgtgagcggataacaattgacattgtgagcggataacaagatactgagcacatcagcaggacgcactgaccgaattcaaaattgaaggcttcaaaaatagataggaggtaagtttatatggattttaatttaacaagagaacaggaactggtccgtcagatggtacgtgaatttgcagaaaacgaggttaaaccgattgctgcagagattgatgagactgaacgcttcccgatggaaaacgtcaaaaagatgggtcagtatggcatgatgggcattccgttctctaaagagtacggcggtgcgggtggcgacgttctgtcttatatcatcgctgtagaggaactgtccaaagtatgtggcaccacgggcgtgatcctgtccgcgcacacctctctgtgcgcaagcctgatcaacgaacacggcaccgaggaacagaagcaaaaatacctggtcccgctggccaaaggtgaaaagatcggtgcatacggtctgacggaaccgaacgcaggtacggacagcggcgcacaacagacggttgcggtactggaaggcgaccactacgttattaacggtagcaaaatcttcatcacgaacggtggcgtggctgacacctttgttatcttcgcgatgaccgaccgcactaaaggcactaaaggtatctctgcgttcatcatcgagaagggtttcaagggtttttctatcggcaaagtggaacagaagctgggtatccgtgcctcctctactaccgagctggttttcgaagacatgattgtgccggttgaaaatatgatcggcaaagaaggcaaaggcttcccgatcgctatgaaaaccctggatggcggccgtatcggcattgcagcacaggcactgggtatcgcagaaggcgctttcaacgaagcacgtgcgtacatgaaagaacgtaaacagtttggccgttctctggataaatttcaaggcctggcgtggatgatggcagacatggacgtagcgattgaatctgcgcgctacctggtctataaagcagcttacctgaaacaggcaggtctgccttacaccgttgacgcagcacgtgcgaaactgcacgcggccaacgttgccatggatgttaccaccaaagccgtgcaactgtttggcggttacggctatactaaggattatccggttgaacgtatgatgcgtgacgcgaaaatcaccgaaatctatgaaggtacttccgaagtgcagaaactggtcatttcaggaaaaatttttagttaattaaaggaggttaagaggatgaatatagttgtttgtttaaaacaggtcccggacaccgcagaagttcgtattgatccagtaaagggtacgctgattcgcgagggcgtgccgtctatcatcaacccagatgacaagaacgccctggaagaagcactggtcctgaaagataattacggcgctcacgtaactgttatctctatgggtccgccgcaagcgaaaaatgcgctggttgaagctctggcgatgggcgctgacgaggctgttctgctgactgatcgtgctttcggtggtgcggacaccctggccacttcccacactatcgcggcaggtatcaagaaactgaaatatgacattgtgtttgctggtcgtcaggctattgacggtgacacggcacaggtaggcccggaaatcgccgaacacctgggtattccgcaggtgacctacgtagaaaaagtagaagtagacggtgataccctgaaaatccgcaaagcatgggaagatggctacgaggtggttgaagtaaaaaccccggtactgctgaccgctatcaaagagctgaatgtaccgcgttacatgtctgttgagaaaatcttcggcgcgttcgacaaggaagtaaagatgtggaccgctgatgatattgacgttgacaaagcgaatctgggcctgaagggctccccaactaaagttaagaagtcctctactaaagaagtgaagggtcagggtgaggtgattgataaacctgttaaagaagctgctgcgtacgtggtttctaagctgaaagaagaacactatatttaagttaggagggatttttcaatgaataaagcagattacaagggcgtttgggtctttgcggaacagcgtgatggtgaactgcagaaagtgtccctggaactgctgggcaaaggcaaggagatggcagaaaaactgggtgttgaactgaccgcagttctgctgggtcacaacactgaaaagatgtccaaagacctgctgtcccatggcgcagacaaggtgctggctgcggacaacgaactgctggctcactttagcaccgacggttatgcaaaagtaatctgcgacctggttaacgagcgcaagccggaaatcctgttcatcggcgccacttttattggtcgcgacctgggccctcgtattgctgcgcgtctgtccactggcctgactgcggattgcacctccctggacattgatgttgaaaaccgtgatctgctggcaactcgcccggcattcggcggcaacctgatcgccaccatcgtatgttccgaccaccgtccgcaaatggctactgtacgtccgggcgtatttgaaaagctgccggtgaacgacgcaaacgtttccgacgacaaaatcgaaaaagttgctatcaagctgaccgctagcgatatccgtaccaaagtttctaaagtagtgaaactggcgaaggacatcgcagatattggtgaagcaaaagttctggtggcaggcggtcgtggcgtcggttccaaagagaacttcgaaaaactggaggaactggcgtctctgctgggcggtactattgcagcgtcccgtgcagcaatcgaaaaagaatgggtggacaaggatctgcaggtgggccagactggtaaaaccgttcgtccgaccctgtacatcgcctgcggcatctccggtgctattcagcacctggccggcatgcaggacagcgactacatcatcgccatcaacaaagacgttgaagctccgatcatgaaagtggcggacctggcaatcgttggtgacgtgaacaaagttgttccggaactgatcgcgcaggttaaagctgctaataattaaggatcccatggtacgcgtgctagaggcatcaaataaaacgaaaggctcagtcgaaagactgggcctttcgttttatctgttgtttgtcggtgaacgctctcctgagtaggacaaatccgccgccctagacctaggcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatga

SEQ ID NO: 12 - DNA, 3206 bp, Artificial Sequence
  Description of Artificial Sequence Synthetic pGV1095 polynucleotide
ctagtgcttggattctcaccaataaaaaacgcccggcggcaaccgagcgttctgaacaaatccagatggagttctgaggtcattactggatctatcaacaggagtccaagcgagctcgatatcaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaactccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccagatatcgacgtctaagaaaccattattatcatgacattaacctataaaaataggcgtatcacgaggccctttcgtcttcacctcgagaaatgtgagcggataacaattgacattgtgagcggataacaagatactgagcacatcagcaggacgcactgaccgaattcaacaggaggggttaaagtggttgatttcgaatattcaataccaactagaatttttttcggtaaagataagataaatgtacttggaagagagcttaaaaaatatggttctaaagtgcttatagtttatggtggaggaagtataaagagaaatggaatatatgataaagctgtaagtatacttgaaaaaaacagtattaaattttatgaacttgcaggagtagagccaaatccaagagtaactacagttgaaaaaggagttaaaatatgtagagaaaatggagttgaagtagtactagctataggtggaggaagtgcaatagattgcgcaaaggttatagcagcagcatgtgaatatgatggaaatccatgggatattgtgttagatggctcaaaaataaaaagggtgcttcctatagctagtatattaaccattgctgcaacaggatcagaaatggatacgtgggcagtaataaataatatggatacaaacgaaaaactaattgcggcacatccagatatggctcctaagttttctatattagatccaacgtatacgtataccgtacctaccaatcaaacagcagcaggaacagctgatattatgagtcatatatttgaggtgtattttagtaatacaaaaacagcatatttgcaggatagaatggcagaagcgttattaagaacttgtattaaatatggaggaatagctcttgagaagccggatgattatgaggcaagagccaatctaatgtgggcttcaagtcttgcgataaatggacttttaacatatggtaaagacactaattggagtgtacacttaatggaacatgaattaagtgcttattacgacataacacacggcgtagggcttgcaattttaacacctaattggatggagtatattttaaataatgatacagtgtacaagtttgttgaatatggtgtaaatgtttggggaatagacaaagaaaaaaatcactatgacatagcacatcaagcaatacaaaaaacaagagattactttgtaaatgtactaggtttaccatctagactgagagatgttggaattgaagaagaaaaattggacataatggcaaaggaatcagtaaagcttacaggaggaaccataggaaacctaagaccagtaaacgcctccgaagtcctacaaatattcaaaaaatctgtgtaaggatcccatggtacgcgtgctagaggcatcaaataaaacgaaaggctcagtcgaaagactgggcctttcgttttatctgttgtttgtcggtgaacgctctcctgagtaggacaaatccgccgccctagacctaggcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatga

SEQ ID NO: 13 - DNA, 2836 bp, Artificial Sequence
  Description of Artificial Sequence Synthetic pGV1094 polynucleotide
ctagtgcttggattctcaccaataaaaaacgcccggcggcaaccgagcgttctgaacaaatccagatggagttctgaggtcattactggatctatcaacaggagtccaagcgagctcgatatcaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaactccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccagatatcgacgtctaagaaaccattattatcatgacattaacctataaaaataggcgtatcacgaggccctttcgtcttcacctcgagaaatgtgagcggataacaattgacattgtgagcggataacaagatactgagcacatcagcaggacgcactgaccgggaattcctatctatttttgaagccttcaatttttcttttctctatgaaagctgtcattgcatccttttgatcctctgttgaaaagcattctccaaatgcttctgattcaaatgctaaagcagtatcaatatcacactgcattcctctattaatagcctgtttgcttaacttaacagctactggagcattgctcacaattttgtttgcaatttcttttgctgtattcattaattcactaggttctactaccttatttacaagtccgattcttaatgcttcatctgcctttatattttgtgcagtaaatataagctgctttgccatgcccattccaactaatcttgaaagtctttgtgtaccaccaaaaccaggtgttattccgagacctacttctggttgaccaaatcttgcgttgcttgaagctattcttatatcacaagacatagctatttcgcatccgcctcctaaagcaaaaccattaacagctgctattacaggcttttcaagaagttctaatcttctaaacactttatttccaagtatcccgaattttctaccttcaatggtattcatttccttcatctcagaaatatctgctcctgctacaaatgatttttctcctgctccagttaaaattactgcaagtacttcgctatcattttcaatttcacctataacataatccatttcttttagtgtatcactatttaacgcatttaatgctttaggtctgttaatggtaactacagcaactttaccttccttttcaaggatgacattgtttagttccatgactaatcctcctaaaatattggatccgatccgatcccatggtacgcgtgctagaggcatcaaataaaacgaaaggctcagtcgaaagactgggcctttcgttttatctgttgtttgtcggtgaacgctctcctgagtaggacaaatccgccgccctagacctaggcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatga

SEQ ID NO: 14 - DNA, 2908 bp, Artificial Sequence
  Description of Artificial Sequence Synthetic pGV1037 polynucleotide
ctagtgcttggattctcaccaataaaaaacgcccggcggcaaccgagcgttctgaacaaatccagatggagttctgaggtcattactggatctatcaacaggagtccaagcgagctcgatatcaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaactccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccagatatcgacgtctaagaaaccattattatcatgacattaacctataaaaataggcgtatcacgaggccctttcgtcttcacctcgagaaatgtgagcggataacaattgacattgtgagcggataacaagatactgagcacatcagcaggacgcactgaccggaattcattgatagtttctttaaatttagggaggtctgtttaatgaaaaaggtatgtgttataggtgcaggtactatgggttcaggaattgctcaggcatttgcagctaaaggatttgaagtagtattaagagatattaaagatgaatttgttgatagaggattagattttatcaataaaaatctttctaaattagttaaaaaaggaaagatagaagaagctactaaagttgaaatcttaactagaatttccggaacagttgaccttaatatggcagctgattgcgatttagttatagaagcagctgttgaaagaatggatattaaaaagcagatttttgctgacttagacaatatatgcaagccagaaacaattcttgcatcaaatacatcatcactttcaataacagaagtggcatcagcaactaaaagacctgataaggttataggtatgcatttctttaatccagctcctgttatgaagcttgtagaggtaataagaggaatagctacatcacaagaaacttttgatgcagttaaagagacatctatagcaataggaaaagatcctgtagaagtagcagaagcaccaggatttgttgtaaatagaatattaataccaatgattaatgaagcagttggtatattagcagaaggaatagcttcagtagaagacatagataaagctatgaaacttggagctaatcacccaatgggaccattagaattaggtgattttataggtcttgatatatgtcttgctataatggatgttttatactcagaaactggagattctaagtatagaccacatacattacttaagaagtatgtaagagcaggatggcttggaagaaaatcaggaaaaggtttctacgattattcaaaataaggatccgatcccatggtacgcgtgctagaggcatcaaataaaacgaaaggctcagtcgaaagactgggcctttcgttttatctgttgtttgtcggtgaacgctctcctgagtaggacaaatccgccgccctagacctaggcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatga

SEQ ID NO: 15 - DNA, 6219 bp, Artificial Sequence
  Description of Artificial Sequence Synthetic pGV1031 polynucleotide
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtaccatatgcataagtttaatttttttgttaaaaaatattaaactttgtgttttttttaacaaaatatattgataaaaataataatagtgggtataattaagttgttagagaaaacgtataaattagggataaactatggaacttatgaaatagattgaaatggtttatctgttaccccgtatcaaaatttaggaggttagttagaatgaaagaagttgtaatagctagtgcagtaagaacagcgattggatcttatggaaagtctcttaaggatgtaccagcagtagatttaggagctacagctataaaggaagcagttaaaaaagcaggaataaaaccagaggatgttaatgaagtcattttaggaaatgttcttcaagcaggtttaggacagaatccagcaagacaggcatcttttaaagcaggattaccagttgaaattccagctatgactattaataaggtttgtggttcaggacttagaacagttagcttagcagcacaaattataaaagcaggagatgctgacgtaataatagcaggtggtatggaaaatatgtctagagctccttacttagcgaataacgctagatggggatatagaatgggaaacgctaaatttgttgatgaaatgatcactgacggattgtgggatgcatttaatgattaccacatgggaataacagcagaaaacatagctgagagatggaacatttcaagagaagaacaagatgagtttgctcttgcatcacaaaaaaaagctgaagaagctataaaatcaggtcaatttaaagatgaaatagttcctgtagtaattaaaggcagaaagggagaaactgtagttgatacagatgagcaccctagatttggatcaactatagaaggacttgcaaaattaaaacctgccttcaaaaaagatggaacagttacagctggtaatgcatcaggattaaatgactgtgcagcagtacttgtaatcatgagtgcagaaaaagctaaagagcttggagtaaaaccacttgctaagatagtttcttatggttcagcaggagttgacccagcaataatgggatatggacctttctatgcaacaaaagcagctattgaaaaagcaggttggacagttgatgaattagatttaatagaatcaaatgaagcttttgcagctcaaagtttagcagtagcaaaagatttaaaatttgatatgaataaagtaaatgtaaatggaggagctattgcccttggtcatccaattggagcatcaggtgcaagaatactcgttactcttgtacacgcaatgcaaaaaagagatgcaaaaaaaggcttagcaactttatgtataggtggcggacaaggaacagcaatattgctagaaaagtgctagaaaggatccagaatttaaaaggagggattaaaatgaactctaaaataattagatttgaaaatttaaggtcattctttaaagatgggatgacaattatgattggaggttttttaaactgtggcactccaaccaaattaattgattttttagttaatttaaatataaagaatttaacgattataagtaatgatacatgttatcctaatacaggtattggtaagttaatatcaaataatcaagtaaaaaagcttattgcttcatatataggcagcaacccagatactggcaaaaaactttttaataatgaacttgaagtagagctctctccccaaggaactctagtggaaagaatacgtgcaggcggatctggcttaggtggtgtactaactaaaacaggtttaggaactttgattgaaaaaggaaagaaaaaaatatctataaatggaacggaatatttgttagagctacctcttacagccgatgtagcattaattaaaggtagtattgtagatgaggccggaaacaccttctataaaggtactactaaaaactttaatccctatatggcaatggcagctaaaaccgtaatagttgaagctgaaaatttagttagctgtgaaaaactagaaaaggaaaaagcaatgacccccggagttcttataaattatatagtaaaggagcctgcataaaatgattaatgataaaaacctagcgaaagaaataatagccaaaagagttgcaagagaattaaaaaatggtcaacttgtaaacttaggtgtaggtcttcctaccatggttgcagattatataccaaaaaatttcaaaattactttccaatcagaaaacggaatagttggaatgggcgctagtcctaaaataaatgaggcagataaagatgtagtaaatgcaggaggagactatacaacagtacttcctgacggcacatttttcgatagctcagtttcgttttcactaatccgtggtggtcacgtagatgttactgttttaggggctctccaggtagatgaaaagggtaatatagccaattggattgttcctggaaaaatgctctctggtatgggtggagctatggatttagtaaatggagctaagaaagtaataattgcaatgagacatacaaataaaggtcaacctaaaattttaaaaaaatgtacacttcccctcacggcaaagtctcaagcaaatctaattgtaacagaacttggagtaattgaggttattaatgatggtttacttctcactgaaattaataaaaacacaaccattgatgaaataaggtctttaactgctgcagatttactcatatccaatgaacttagacccatggctgtttagaaagaattcttgatatcaggaaggtgacttttatgttaaaggatgaagtaattaaacaaattagcacgccattaacttcgcctgcatttcctagaggaccctataaatttcataatcgtgagtattttaacattgtatatcgtacagatatggatgctcttcgtaaagttgtgccagagcctttagaaattgatgagcccttagtcaggtttgaaattatggcaatgcatgatacgagtggacttggttgttatacagaaagcggacaggctattcccgtaagctgtaatggagttaagggagattatcttcatatgatgtatttagataatgagcctgcaattgcagtaggaagggaattaagtgcatatcctaaaaagctcgggtatccaaagctttttgtggattcagatactttagtaggaactttagactatggaaaacttagagttgcgacagctacaatggggtacaaacataaagccttagatgctaatgaagcaaaggatcaaatttgtcgccctaattatatgttgaaaataatacccaattatgatggaagccctaggatatgtgagcttataaatgcgaaaatcacagatgttaccgtacatgaagcttggacaggaccaactcgactgcagttatttgatcacgctatggcgccacttaatgatttgccagtaaaagagattgtttctagctctcacattcttgcagatataatattgcctagagctgaagttatatatgattatcttaagtaataaaaataagagttaccttaaatggtaactcttatttttttaatgtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 16 - DNA, 2855 bp, Artificial Sequence
  Description of Artificial Sequence Synthetic pGV1049 polynucleotide
ctagtgcttggattctcaccaataaaaaacgcccggcggcaaccgagcgttctgaacaaatccagatggagttctgaggtcattactggatctatcaacaggagtccaagcgagctcgatatcaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaactccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccagatatcgacgtctaagaaaccattattatcatgacattaacctataaaaataggcgtatcacgaggccctttcgtcttcacctcgagaaatgtgagcggataacaattgacattgtgagcggataacaagatactgagcacatcagcaggacgcactgaccgaattcattaaagaggagaaaggtaccaaaataagcaagtttgaaggaggtccttagaatggaattaaaaaatgttattcttgaaaaagaagggcatttagctattgttacaatcaatagaccaaaggcattaaatgcattgaattcagaaacactaaaagatttaaatgttgttttagatgatttagaagcagacaacaatgtgtatgcagttatagttacaggtgctggtgagaaatcttttgttgctggagcagatatttcagaaatgaaagatcttaatgaagaacaaggtaaagaatttggtattttaggaaacaatgtcttcagaagattagaaaaattggataagccagttatcgcagctatatcaggatttgctcttggtggtggatgtgaacttgctatgtcatgtgacataagaatagcttcagttaaagctaaatttggtcaaccagaagcaggacttggaataactccaggatttggtggaactcaaagattagctagaattgtagggccaggaaaagctaaagaattaatttatacttgtgaccttataaatgcagaagaagcttatagaataggtttagttaataaagtagttgaattagaaaaattgatggaagaagcaaaagcaatggctaacaagattgcagctaatgctccaaaagcagttgcatattgtaaagatgctatagacagaggaatgcaagttgatatagatgcagctatattaatagaagcagaagactttggaaagtgctttgcaacagaagatcaaacagaaggaatgactgcgttcttagaaagaagagcagaaaagaattttcaaaataaataaggatcccatggtacgcgtgctagaggcatcaaataaaacgaaaggctcagtcgaaagactgggcctttcgttttatctgttgtttgtcggtgaacgctctcctgagtaggacaaatccgccgccctagacctaggcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatga

SEQ ID NO: 17 - DNA, 2891 bp, Artificial Sequence
  Description of Artificial Sequence Synthetic pGV1050 polynucleotide
ctagtgcttggattctcaccaataaaaaacgcccggcggcaaccgagcgttctgaacaaatccagatggagttctgaggtcattactggatctatcaacaggagtccaagcgagctcgatatcaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaactccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccagatatcgacgtctaagaaaccattattatcatgacattaacctataaaaataggcgtatcacgaggccctttcgtcttcacctcgagaaatgtgagcggataacaattgacattgtgagcggataacaagatactgagcacatcagcaggacgcactgaccgaattcaaaagatttagaggaggaataattcatgaaaaagatttttgtacttggagcaggaacaatgggtgctggtatcgttcaagcattcgctcaaaaaggttgtgaagtaattgtaagagacataaaggaagaatttgttgacagaggaatagctggaatcactaaaggattagaaaagcaagttgctaaaggaaaaatgtctgaagaagataaagaagctatactttcaagaatttcaggaacaactgatatgaaattagctgctgactgtgatttagtagttgaagctgcaatcgaaaacatgaaaattaagaaggaaatcttcgctgaattagatggaatttgtaagccagaagcgattttagcttcaaacacttcatctttatcaattactgaagttgcttcagctacaaagagacctgataaagttatcggaatgcatttctttaatccagctccagtaatgaagcttgttgaaattattaaaggaatagctacttctcaagaaacttttgatgctgttaaggaattatcagttgctattggaaaagaaccagtagaagttgcagaagctccaggattcgttgtaaacagaatattaatcccaatgattaacgaagcttcatttatcctacaagaaggaatagcttcagttgaagatattgatacagctatgaaatatggtgctaaccatccaatgggacctttagctttaggagatcttattggattagacgtttgcttagctatcatggatgttttattcactgaaacaggtgataacaagtacagagctagcagcatattaagaaaatatgttagagctggatggcttggaagaaaatcaggaaaaggattctatgattattctaaataaggatcccatggtacgcgtgctagaggcatcaaataaaacgaaaggctcagtcgaaagactgggcctttcgttttatctgttgtttgtcggtgaacgctctcctgagtaggacaaatccgccgccctagacctaggcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatga

SEQ ID NO: 18 - DNA, 3205 bp, Artificial Sequence
  Description of Artificial Sequence Synthetic pGV1091 polynucleotide
ctagtgcttggattctcaccaataaaaaacgcccggcggcaaccgagcgttctgaacaaatccagatggagttctgaggtcattactggatctatcaacaggagtccaagcgagctcgatatcaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaactccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccagatatcgacgtctaagaaaccattattatcatgacattaacctataaaaataggcgtatcacgaggccctttcgtcttcacctcgagaaatgtgagcggataacaattgacattgtgagcggataacaagatactgagcacatcagcaggacgcactgaccgaattcattaaagaggagaaaggtaccatggcacgttttactttaccaagagacatttatcatggagaaggagcacttgaggcacttaaaactttaaaaggtaagaaagctttcttagtagttggtggcggatcaatgaaaagatttggatttcttaaacaagttgaagattatttaaaagaagcaggaatggaagtagaattatttgaaggtgttgaaccagatccatcagtggaaacagtaatgaaaggcgcagaagctatgagaaactttgagcctgattggatagttgcaatgggtggaggatcaccaattgatgctgcaaaggctatgtggatattctacgaatacccagattttacttttgaacaagcagttgttccatttggattaccagaccttagacaaaaagctaagtttgtagctattccatcaacaagcggtacagctacagaagttacagcattctcagttatcacaaattattcagaaaaaattaaatatcctttagctgattttaacataactccagatatagcaatagttgatccagcacttgctcaaactatgccaaaaactttaacagctcatactggaatggatgcattaactcacgctatagaagcatacactgcatcacttcaatcaaatttctcagatccattagcaattaaagctgtagaaatggttcaagaaaatttaatcaaatcatttgaaggagataaagaagctagaaatctaatgcatgaagctcaatgtttagctggaatggcattttctaatgcattacttggaatagttcactcaatggctcataaggttggtgctgtattccatattcctcatggatgtgcaaatgctatatttttaccatatgtaattgagtataacagaacaaaatgcgaaaatagatatggagatattgcgagagccttaaaattaaaaggaaacaatgatgccgagttaactgattcattaattgaattaattaatggattaaatgataagttagagattcctcactcaatgaaagagtatggagttactgaagaagattttaaagctaatctttcatttatcgctcataacgcagtattagatgcatgcacaggatcaaatcctagagaaatagatgatgctacaatggaaaaattatttgaatgcacatactatggaactaaagttaatttgtaaggatcccatggtacgcgtgctagaggcatcaaataaaacgaaaggctcagtcgaaagactgggcctttcgttttatctgttgtttgtcggtgaacgctctcctgagtaggacaaatccgccgccctagacctaggcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatga

SEQ ID NO: 19 - DNA, 3449 bp, Artificial Sequence
  Description of Artificial Sequence Synthetic pGV1096 polynucleotide
ctagtgcttggattctcaccaataaaaaacgcccggcggcaaccgagcgttctgaacaaatccagatggagttctgaggtcattactggatctatcaacaggagtccaagcgagctcgatatcaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaactccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccagatatcgacgtctaagaaaccattattatcatgacattaacctataaaaataggcgtatcacgaggccctttcgtcttcacctcgagaaatgtgagcggataacaattgacattgtgagcggataacaagatactgagcacatcagcaggacgcactgaccgggaattcggaggaatagttcatgaataaagacacactaatacctacaactaaagatttaaaagtaaaaacaaatggtgaaaacattaatttaaagaactacaaggataattcttcatgtttcggagtattcgaaaatgttgaaaatgctataagcagcgctgtacacgcacaaaagatattatcccttcattatacaaaagagcaaagagaaaaaatcataactgagataagaaaggccgcattacaaaataaagaggtcttggctacaatgattctagaagaaacacatatgggaagatatgaggataaaatattaaaacatgaattggtagctaaatatactcctggtacagaagatttaactactactgcttggtcaggtgataatggtcttacagttgtagaaatgtctccatatggtgttataggtgcaataactccttctacgaatccaactgaaactgtaatatgtaatagcataggcatgatagctgctggaaatgctgtagtatttaacggacacccatgcgctaaaaaatgtgttgcctttgctgttgaaatgataaataaggcaattatttcatgtggcggtcctgaaaatctagtaacaactataaaaaatccaactatggagtctctagatgcaattattaagcatccttcaataaaacttctttgcggaactgggggtccaggaatggtaaaaaccctcttaaattctggtaagaaagctataggtgctggtgctggaaatccaccagttattgtagatgatactgctgatatagaaaaggctggtaggagcatcattgaaggctgttcttttgataataatttaccttgtattgcagaaaaagaagtatttgtttttgagaatgttgcagatgatttaatatctaacatgctaaaaaataatgctgtaattataaatgaagatcaagtatcaaaattaatagatttagtattacaaaaaaataatgaaactcaagaatactttataaacaaaaaatgggtaggaaaagatgcaaaattattcttagatgaaatagatgttgagtctccttcaaatgttaaatgcataatctgcgaagtaaatgcaaatcatccatttgttatgacagaactcatgatgccaatattgccaattgtaagagttaaagatatagatgaagctattaaatatgcaaagatagcagaacaaaatagaaaacatagtgcctatatttattctaaaaatatagacaacctaaatagatttgaaagagaaatagatactactatttttgtaaagaatgctaaatcttttgctggtgttggttatgaagcagaaggatttacaactttcactattgctggatctactggtgagggaataacctctgcaaggaattttacaagacaaagaagatgtgtacttgccggctaaggatccgatccgatcccatggtacgcgtgctagaggcatcaaataaaacgaaaggctcagtcgaaagactgggcctttcgttttatctgttgtttgtcggtgaacgctctcctgagtaggacaaatccgccgccctagacctaggcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatga

SEQ ID NO: 20 - DNA, 1425 bp, Artificial Sequence
  Description of Artificial Sequence Synthetic lpdA polynucleotide
atgagtactgaaatcaaaactcaggtcgtggtacttggggcaggccccgcaggttactccgctgccttccgttgcgctgatttaggtctggaaaccgtaatcgtagaacgttacaacacccttggcggtgtttgcctgaacgtcggctgtatcccttctaaagcactgctgcacgtagcaaaagttatcgaagaagccaaagcgctggctgaacacggtatcgtcttcggcgaaccgaaaaccgatatcgacaagattcgtacctggaaagagaaagtgatcaatcagctgaccggtggtctggctggtatggcgaaaggccgcaaagtcaaagtggtcaacggtctgggtaaattcaccggggctaacaccctggaagttgaaggtgagaacggcaaaaccgtgatcaacttcgacaacgcgatcattgcagcgggttctcgcccgatccaactgccgtttattccgcatgaagatccgcgtatctgggactccactgacgcgctggaactgaaagaagtaccagaacgcctgctggtaatgggtggcggtatcatcggtctggaaatgggcaccgtttaccacgcgctgggttcacagattgacgtggttgaaatgttcgaccaggttatcccggcagctgacaaagacatcgttaaagtcttcaccaagcgtatcagcaagaaattcaacctgatgctggaaaccaaagttaccgccgttgaagcgaaagaagacggcatttatgtgacgatggaaggcaaaaaagcacccgctgaaccgcagcgttacgacgccgtgctggtagcgattggtcgtgtgccgaacggtaaaaacctcgacgcaggcaaagcaggcgtggaagttgacgaccgtggtttcatccgcgttgacaaacagctgcgtaccaacgtaccgcacatctttgctatcggcgatatcgtcggtcaaccgatgctggcacacaaaggtgttcacgaaggtcacgttgccgctgaagttatcgccggtaagaaacactacttcgatccgaaagttatcccgtccatcgcctataccgaaccagaagttgcatgggtgggtctgactgagaaagaagcgaaagagaaaggcatcagctatgaaaccgccaccttcccgtgggctgcttctggtcgtgctatcgcttccgactgcgcagacggtatgaccaagctgattttcgacaaagaatctcaccgtgtgatcggtggtgcgattgtcggtactaacggcggcgagctgctgggtgaaatcggcctggcaatcgaaatgggttgtgatgctgaagacatcgcactgaccatccacgcgcacccgactctgcacgagtctgtgggcctggcggcagaagtgttcgaaggtagcattaccgacctgccgaacccgaaagcgaagaagaagtaa

SEQ ID NO: 21 - DNA, 2664 bp, Artificial Sequence
  Description of Artificial Sequence Synthetic aceE polynucleotide
atgtcagaacgtttcccaaatgacgtggatccgatcgaaactcgcgactggctccaggcgatcgaatcggtcatccgtgaagaaggtgttgagcgtgctcagtatctgatcgaccaactgcttgctgaagcccgcaaaggcggtgtaaacgtagccgcaggcacaggtatcagcaactacatcaacaccatccccgttgaagaacaaccggagtatccgggtaatctggaactggaacgccgtattcgttcagctatccgctggaacgccatcatgacggtgctgcgtgcgtcgaaaaaagacctcgaactgggcggccatatggcgtccttccagtcttccgcaaccatttatgatgtgtgctttaaccacttcttccgtgcacgcaacgagcaggatggcggcgacctggtttacttccagggccacatctccccgggcgtgtacgctcgtgctttcctggaaggtcgtctgactcaggagcagctggataacttccgtcaggaagttcacggcaatggcctctcttcctatccgcacccgaaactgatgccggaattctggcagttcccgaccgtatctatgggtctgggtccgattggtgctatttaccaggctaaattcctgaaatatctggaacaccgtggcctgaaagatacctctaaacaaaccgtttacgcgttcctcggtgacggtgaaatggacgaaccggaatccaaaggtgcgatcaccatcgctacccgtgaaaaactggataacctggtcttcgttatcaactgtaacctgcagcgtcttgacggcccggtcaccggtaacggcaagatcatcaacgaactggaaggcatcttcgaaggtgctggctggaacgtgatcaaagtgatgtggggtagccgttgggatgaactgctgcgtaaggataccagcggtaaactgatccagctgatgaacgaaaccgttgacggcgactaccagaccttcaaatcgaaagatggtgcgtacgttcgtgaacacttcttcggtaaatatcctgaaaccgcagcactggttgcagactggactgacgagcagatctgggcactgaaccgtggtggtcacgatccgaagaaaatctacgctgcattcaagaaagcgcaggaaaccaaaggcaaagcgacagtaatccttgctcataccattaaaggttacggcatgggcgacgcggctgaaggtaaaaacatcgcgcaccaggttaagaaaatgaacatggacggtgtgcgtcatatccgcgaccgtttcaatgtgccggtgtctgatgcagatatcgaaaaactgccgtacatcaccttcccggaaggttctgaagagcatacctatctgcacgctcagcgtcagaaactgcacggttatctgccaagccgtcagccgaacttcaccgagaagcttgagctgccgagcctgcaagacttcggcgcgctgttggaagagcagagcaaagagatctctaccactatcgctttcgttcgtgctctgaacgtgatgctgaagaacaagtcgatcaaagatcgtctggtaccgatcatcgccgacgaagcgcgtactttcggtatggaaggtctgttccgtcagattggtatttacagcccgaacggtcagcagtacaccccgcaggaccgcgagcaggttgcttactataaagaagacgagaaaggtcagattctgcaggaagggatcaacgagctgggcgcaggttgttcctggctggcagcggcgacctcttacagcaccaacaatctgccgatgatcccgttctacatctattactcgatgttcggcttccagcgtattggcgatctgtgctgggcggctggcgaccagcaagcgcgtggcttcctgatcggcggtacttccggtcgtaccaccctgaacggcgaaggtctgcagcacgaagatggtcacagccacattcagtcgctgactatcccgaactgtatctcttacgacccggcttacgcttacgaagttgctgtcatcatgcatgacggtctggagcgtatgtacggtgaaaaacaagagaacgtttactactacatcactacgctgaacgaaaactaccacatgccggcaatgccggaaggtgctgaggaaggtatccgtaaaggtatctacaaactcgaaactattgaaggtagcaaaggtaaagttcagctgctcggctccggttctatcctgcgtcacgtccgtgaagcagctgagatcctggcgaaagattacggcgtaggttctgacgtttatagcgtgacctccttcaccgagctggcgcgtgatggtcaggattgtgaacgctggaacatgctgcacccgctggaaactccgcgcgttccgtatatcgctcaggtgatgaacgacgctccggcagtggcatctaccgactatatgaaactgttcgctgagcaggtccgtacttacgtaccggctgacgactaccgcgtactgggtactgatggcttcggtcgttccgacagccgtgagaacctgcgtcaccacttcgaagttgatgcttcttatgtcgtggttgcggcgctgggcgaactggctaaacgtggcgaaatcgataagaaagtggttgctgacgcaatcgccaaattcaacatcgatgcagataaagttaacccgcgtctggcgtaa

SEQ ID NO: 22 - DNA, 1893 bp, Artificial Sequence
  Description of Artificial Sequence Synthetic aceF polynucleotide
atggctatcgaaatcaaagtaccggacatcggggctgatgaagttgaaatcaccgagatcctggtcaaagtgggcgacaaagttgaagccgaacagtcgctgatcaccgtagaaggcgacaaagcctctatggaagttccgtctccgcaggcgggtatcgttaaagagatcaaagtctctgttggcgataaaacccagaccggcgcactgattatgattttcgattccgccgacggtgcagcagacgctgcacctgctcaggcagaagagaagaaagaagcagctccggcagcagcaccagcggctgcggcggcaaaagacgttaacgttccggatatcggcagcgacgaagttgaagtgaccgaaatcctggtgaaagttggcgataaagttgaagctgaacagtcgctgatcaccgtagaaggcgacaaggcttctatggaagttccggctccgtttgctggcaccgtgaaagagatcaaagtgaacgtgggtgacaaagtgtctaccggctcgctgattatggtcttcgaagtcgcgggtgaagcaggcgcggcagctccggccgctaaacaggaagcagctccggcagcggcccctgcaccagcggctggcgtgaaagaagttaacgttccggatatcggcggtgacgaagttgaagtgactgaagtgatggtgaaagtgggcgacaaagttgccgctgaacagtcactgatcaccgtagaaggcgacaaagcttctatggaagttccggcgccgtttgcaggcgtcgtgaaggaactgaaagtcaacgttggcgataaagtgaaaactggctcgctgattatgatcttcgaagttgaaggcgcagcgcctgcggcagctcctgcgaaacaggaagcggcagcgccggcaccggcagcaaaagctgaagccccggcagcagcaccagctgcgaaagcggaaggcaaatctgaatttgctgaaaacgacgcttatgttcacgcgactccgctgatccgccgtctggcacgcgagtttggtgttaaccttgcgaaagtgaagggcactggccgtaaaggtcgtatcctgcgcgaagacgttcaggcttacgtgaaagaagctatcaaacgtgcagaagcagctccggcagcgactggcggtggtatccctggcatgctgccgtggccgaaggtggacttcagcaagtttggtgaaatcgaagaagtggaactgggccgcatccagaaaatctctggtgcgaacctgagccgtaactgggtaatgatcccgcatgttactcacttcgacaaaaccgatatcaccgagttggaagcgttccgtaaacagcagaacgaagaagcggcgaaacgtaagctggatgtgaagatcaccccggttgtcttcatcatgaaagccgttgctgcagctcttgagcagatgcctcgcttcaatagttcgctgtcggaagacggtcagcgtctgaccctgaagaaatacatcaacatcggtgtggcggtggataccccgaacggtctggttgttccggtattcaaagacgtcaacaagaaaggcatcatcgagctgtctcgcgagctgatgactatttctaagaaagcgcgtgacggtaagctgactgcgggcgaaatgcagggcggttgcttcaccatctccagcatcggcggcctgggtactacccacttcgcgccgattgtgaacgcgccggaagtggctatcctcggcgtttccaagtccgcgatggagccggtgtggaatggtaaagagttcgtgccgcgtctgatgctgccgatttctctctccttcgaccaccgcgtgatcgacggtgctgatggtgcccgtttcattaccatcattaacaacacgctgtctgacattcgccgtctggtgatgtaa

SEQ ID NO: 23 - DNA, 1263 bp, Artificial Sequence
  Description of Artificial Sequence Synthetic PDA1 polynucleotide
atgcttgctgcttcattcaaacgccaaccatcacaattggtccgcgggttaggagctgttcttcgcactcccaccaggataggtcatgttcgtaccatggcaactttaaaaacaactgataagaaggcccctgaggacatcgagggctcggacacagtgcaaattgagttgcctgaatcttccttcgagtcgtatatgctagagcctccagacttgtcttatgagacttcgaaagccaccttgttacagatgtataaagatatggtcatcatcagaagaatggagatggcttgtgacgccttgtacaaggccaagaaaatcagaggtttttgccatctatctgttggtcaggaggccattgctgtcggtatcgagaatgccatcacaaaattggattccatcatcacatcttacagatgtcacggtttcacttttatgagaggtgcctcagtgaaagccgttctggctgaattgatgggtagaagagccggtgtctcttatggtaagggtggttccatgcacctttacgctccaggcttctatggtggtaatggtatcgtgggtgcccaggttcctttaggtgcaggtttagcttttgctcaccaatacaagaacgaggacgcctgctctttcactttgtatggtgatggtgcctctaatcaaggtcaagtttttgaatctttcaacatggccaaattatggaatttgcccgtcgtgttttgctgtgagaacaacaagtacggtatgggtaccgccgcttcaagatcctccgcgatgactgaatatttcaagcgtggtcaatatattccaggtttaaaagttaacggtatggatattctagctgtctaccaagcatccaagtttgctaaggactggtgtctatccggcaaaggtcctctcgttctagaatatgaaacctataggtacggtggccattctatgtctgatcccggtactacctacagaactagagacgagattcagcatatgagatccaagaacgatccaattgctggtcttaagatgcatttgattgatctaggtattgccactgaagctgaagtcaaagcttacgacaagtccgctagaaaatacgttgacgaacaagttgaattagctgatgctgctcctcctccagaagccaaattatccatcttgtttgaagacgtctacgtgaaaggtacagaaactccaaccctaagaggtaggatccctgaagatacttgggacttcaaaaagcaaggttttgcctctagggattaa

SEQ ID NO: 24 - DNA, 1101 bp, Artificial Sequence
  Description of Artificial Sequence Synthetic PDB1 polynucleotide
atgttttccagactgccaacatcattggccagaaatgttgcacgtcgtgccccaacttcttttgtaagaccctctgcagcagcagcagcattgagattctcatcaacaaagacgatgaccgtcagagaggccttgaatagtgccatggcggaagaattggaccgtgatgatgatgtcttccttattggtgaagaagttgcacaatataacggggcttataaggtgtcaaagggtttattggacaggttcggtgaacgtcgtgtggttgacacacctattaccgaatacgggttcacaggtttggccgttggtgccgctttgaagggtttgaagccaattgtagagtttatgtcgttcaatttctctatgcaagctatcgatcatgttgtcaattccgctgcaaagactcactacatgtctggtggtactcaaaaatgtcaaatggtcttcagaggtcctaatggtgctgcagtgggtgttggtgctcaacattcacaggacttttctccttggtacggttccattccagggttaaaggtccttgtcccttattctgctgaagatgctaggggtttgttaaaggccgccatcagagatccaaaccctgttgtatttttagagaacgaattgttgtacggtgaatcttttgaaatctcagaagaagctttatcccctgagttcaccttgccatacaaggctaagatcgaaagagaaggtaccgatatttccattgttacgtacacaagaaacgttcagttttctttggaagccgctgaaattctacaaaagaaatatggtgtctctgcagaagttatcaacttgcgttctattagacctttagatactgaagctatcatcaaaactgtcaagaagacaaaccacttgattactgttgaatccactttcccatcatttggtgttggtgctgaaattgtcgcccaagttatggagtctgaagcctttgattacttggatgctccaatccaaagagttactggtgccgatgttccaacaccttacgctaaagaattagaagatttcgctttccctgatactccaaccatcgttaaagctgtcaaagaagtcttgtcaattgaataa

SEQ ID NO: 25 - DNA, 1233 bp, Artificial Sequence
  Description of Artificial Sequence Synthetic PDX1 polynucleotide
atgctaagtgcaatttccaaagtctccactttaaaatcatgtacaagatatttaaccaaatgcaactatcatgcatcagctaaattacttgctgtaaagacattttcaatgcctgcaatgtctcctactatggagaaaggggggattgtgtcttggaaatataaagttggcgaaccattcagcgcgggcgatgtgatattagaagtggaaacagataaatctcaaattgatgtggaagcactggacgatggtaaactagctaagatcctgaaagatgaaggctctaaagatgttgatgttggtgaacctattgcttatattgctgatgttgatgatgatttagctactataaagttaccccaagaggccaacaccgcaaatgcgaaatctattgaaattaagaagccatccgcagatagtactgaagcaacacaacaacatttaaaaaaagccacagttacaccaataaaaaccgttgacggcagccaagccaatcttgaacagacgctattaccatccgtgtcattactactggctgagaacaatatatccaaacaaaaggctttgaaggaaattgcgccatctggttccaacggtagactattaaagggtgatgtgctagcatacctagggaaaataccacaagattcggttaacaaggtaacagaatttatcaagaagaacgaacgtctcgatttatcgaacattaaacctatacagctcaaaccaaaaatagccgagcaagctcaaacaaaagctgccgacaagccaaagattactcctgtagaatttgaagagcaattagtgttccatgctcccgcctctattccgtttgacaaactgagtgaatcattgaactctttcatgaaagaagcttaccagttctcacacggaacaccactaatggacacaaattcgaaatactttgaccctattttcgaggaccttgtcaccttgagcccaagagagccaagatttaaattttcctatgacttgatgcaaattcccaaagctaataacatgcaagacacgtacggtcaagaagacatatttgacctcttaacaggttcagacgcgactgcctcatcagtaagacccgttgaaaagaacttacctgaaaaaaacgaatatatactagcgttgaatgttagcgtcaacaacaagaagtttaatgacgcggaggccaaggcaaaaagattccttgattacgtaagggagttagaatcattttga

SEQ ID NO: 26 - DNA, 1449 bp, Artificial Sequence
  Description of Artificial Sequence Synthetic LAT1 polynucleotide
atgtctgcctttgtcagggtggttccaagaatatccagaagttcagtactcaccagatcattgagactgcaattgagatgctacgcatcgtacccagagcacaccattattggtatgccggcactgtctcctacgatgacgcaaggtaatcttgctgcttggactaagaaggaaggtgaccaattgtctcccggtgaagttattgccgaaatagaaacagacaaggctcaaatggactttgagttccaagaagatggttacttagccaagattctagttcctgaaggtacaaaggacattcctgtcaacaagcctattgccgtctatgtggaggacaaagctgatgtgccagcttttaaggactttaagctggaggattcaggttctgattcaaagaccagtacgaaggctcagcctgccgaaccacaggcagaaaagaaacaagaagcgccagctgaagagaccaagacttctgcacctgaagctaagaaatctgacgttgctgctcctcaaggtaggatttttgcctctccacttgccaagactatcgccttggaaaagggtatttctttgaaggatgttcacggcactggaccccgcggtagaattaccaaggctgacattgagtcatatctagaaaagtcgtctaagcagtcttctcaaaccagtggtgctgccgccgccactcctgccgccgctacctcaagcactactgctggctctgctccatcgccttcttctacagcatcatatgaggatgttccaatttcaaccatgagaagcatcattggagaacgtttattgcaatctactcaaggcattccatcatacatcgtttcctccaagatatccatctccaaacttttgaaattgagacagtccttgaacgctacagcaaacgacaagtacaaactgtccattaatgacctattagtaaaagccatcactgttgcggctaagagggtgccagatgccaatgcctactggttacctaatgagaacgttatccgtaaattcaagaatgtcgatgtctcagtcgctgttgccacaccaacaggattattgacaccaattgtcaagaattgtgaggccaagggcttgtcgcaaatctctaacgaaatcaaggaactagtcaagcgtgccagaataaacaaattggcaccagaggaattccaaggtgggaccatttgcatatccaatatgggcatgaataatgctgttaacatgtttacttcgattatcaacccaccacagtctacaatcttggccatcgctactgttgaaagggtcgctgtggaagacgccgctgctgagaacggattctcctttgataaccaggttaccataacagggacctttgatcatagaaccattgatggcgccaaaggtgcagaattcatgaaggaattgaaaactgttattgaaaatcctttggaaatgctattgtga

SEQ ID NO: 27 - DNA, 1500 bp, Artificial Sequence
  Description of Artificial Sequence Synthetic LPD1 polynucleotide
atgttaagaatcagatcactcctaaataataagcgtgccttttcgtccacagtcaggacattgaccattaacaagtcacatgatgtagtcatcatcggtggtggccctgctggttacgtggctgctatcaaagctgctcaattgggatttaacactgcatgtgtagaaaaaagaggcaaattaggcggtacctgtcttaacgttggatgtatcccctccaaagcacttctaaataattctcatttattccaccaaatgcatacggaagcgcaaaagagaggtattgacgtcaacggtgatatcaaaattaacgtagcaaacttccaaaaggctaaggatgacgctgttaagcaattaactggaggtattgagcttctgttcaagaaaaataaggtcacctattataaaggtaatggttcattcgaagacgaaacgaagatcagagtaactcccgttgatgggttggaaggcactgtcaaggaagaccacatactagatgttaagaacatcatagtcgccacgggctctgaagttacacccttccccggtattgaaatagatgaggaaaaaattgtctcttcaacaggtgctctttcgttaaaggaaattcccaaaagattaaccatcattggtggaggaatcatcggattggaaatgggttcagtttactctagattaggctccaaggttactgtagtagaatttcaacctcaaattggtgcatctatggacggcgaggttgccaaagccacccaaaagttcttgaaaaagcaaggtttggacttcaaattaagcaccaaagttatttctgcaaagagaaacgacgacaagaacgtcgtcgaaattgttgtagaagatactaaaacgaataagcaagaaaatttggaagctgaagttttgctggttgctgttggtagaagaccttacattgctggcttaggggctgaaaagattggattagaagtagacaaaaggggacgcctagtcattgatgaccaatttaattccaagttcccacacattaaagtggtaggagatgttacatttggtccaatgctggctcacaaagccgaagaggaaggtattgcagctgtcgaaatgttgaaaactggtcacggtcatgtcaactataacaacattccttcggtcatgtattctcacccagaagtagcatgggttggtaaaaccgaagagcaattgaaagaagccggcattgactataaaattggtaagttcccctttgcggccaattcaagagccaagaccaaccaagacactgaaggtttcgtgaagattttgatcgattccaagaccgagcgtattttgggggctcacattatcggtccaaatgccggtgaaatgattgctgaagctggcttagccttagaatatggcgcttccgcagaagatgttgctagggtctgccatgctcatcctactttgtccgaagcatttaaggaagctaacatggctgcctatgataaagctattcattgttga

SEQ ID NO: 28 - DNA, 1692 bp, Artificial Sequence
  Description of Artificial Sequence Synthetic PDC1 polynucleotide
atgtctgaaattactttgggtaaatatttgttcgaaagattaaagcaagtcaacgttaacaccgttttcggtttgccaggtgacttcaacttgtccttgttggacaagatctacgaagttgaaggtatgagatgggctggtaacgccaacgaattgaacgctgcttacgccgctgatggttacgctcgtatcaagggtatgtcttgtatcatcaccaccttcggtgtcggtgaattgtctgctttgaacggtattgccggttcttacgctgaacacgtcggtgttttgcacgttgttggtgtcccatccatctctgctcaagctaagcaattgttgttgcaccacaccttgggtaacggtgacttcactgttttccacagaatgtctgccaacatttctgaaaccactgctatgatcactgacattgctaccgccccagctgaaattgacagatgtatcagaaccacttacgtcacccaaagaccagtctacttaggtttgccagctaacttggtcgacttgaacgtcccagctaagttgttgcaaactccaattgacatgtctttgaagccaaacgatgctgaatccgaaaaggaagtcattgacaccatcttggctttggtcaaggatgctaagaacccagttatcttggctgatgcttgttgttccagacacgacgtcaaggctgaaactaagaagttgattgacttgactcaattcccagctttcgtcaccccaatgggtaagggttccattgacgaacaacacccaagatacggtggtgtttacgtcggtaccttgtccaagccagaagttaaggaagccgttgaatctgctgacttgattttgtctgtcggtgctttgttgtctgatttcaacaccggttctttctcttactcttacaagaccaagaacattgtcgaattccactccgaccacatgaagatcagaaacgccactttcccaggtgtccaaatgaaattcgttttgcaaaagttgttgaccactattgctgacgccgctaagggttacaagccagttgctgtcccagctagaactccagctaacgctgctgtcccagcttctaccccattgaagcaagaatggatgtggaaccaattgggtaacttcttgcaagaaggtgatgttgtcattgctgaaaccggtacctccgctttcggtatcaaccaaaccactttcccaaacaacacctacggtatctctcaagtcttatggggttccattggtttcaccactggtgctaccttgggtgctgctttcgctgctgaagaaattgatccaaagaagagagttatcttattcattggtgacggttctttgcaattgactgttcaagaaatctccaccatgatcagatggggcttgaagccatacttgttcgtcttgaacaacgatggttacaccattgaaaagttgattcacggtccaaaggctcaatacaacgaaattcaaggttgggaccacctatccttgttgccaactttcggtgctaaggactatgaaacccacagagtcgctaccaccggtgaatgggacaagttgacccaagacaagtctttcaacgacaactctaagatcagaatgattgaaatcatgttgccagtcttcgatgctccacaaaacttggttgaacaagctaagttgactgctgctaccaacgctaagcaataa

SEQ ID NO: 29 - DNA, 1503 bp, Artificial Sequence
  Description of Artificial Sequence Synthetic ALD6 polynucleotide
atgactaagctacactttgacactgctgaaccagtcaagatcacacttccaaatggtttgacatacgagcaaccaaccggtctattcattaacaacaagtttatgaaagctcaagacggtaagacctatcccgtcgaagatccttccactgaaaacaccgtttgtgaggtctcttctgccaccactgaagatgttgaatatgctatcgaatgtgccgaccgtgctttccacgacactgaatgggctacccaagacccaagagaaagaggccgtctactaagtaagttggctgacgaattggaaagccaaattgacttggtttcttccattgaagctttggacaatggtaaaactttggccttagcccgtggggatgttaccattgcaatcaactgtctaagagatgctgctgcctatgccgacaaagtcaacggtagaacaatcaacaccggtgacggctacatgaacttcaccaccttagagccaatcggtgtctgtggtcaaattattccatggaactttccaataatgatgttggcttggaagatcgccccagcattggccatgggtaacgtctgtatcttgaaacccgctgctgtcacacctttaaatgccctatactttgcttctttatgtaagaaggttggtattccagctggtgtcgtcaacatcgttccaggtcctggtagaactgttggtgctgctttgaccaacgacccaagaatcagaaagctggcttttaccggttctacagaagtcggtaagagtgttgctgtcgactcttctgaatctaacttgaagaaaatcactttggaactaggtggtaagtccgcccatttggtctttgacgatgctaacattaagaagactttaccaaatctagtaaacggtattttcaagaacgctggtcaaatttgttcctctggttctagaatttacgttcaagaaggtatttacgacgaactattggctgctttcaaggcttacttggaaaccgaaatcaaagttggtaatccatttgacaaggctaacttccaaggtgctatcactaaccgtcaacaattcgacacaattatgaactacatcgatatcggtaagaaagaaggcgccaagatcttaactggtggcgaaaaagttggtgacaagggttacttcatcagaccaaccgttttctacgatgttaatgaagacatgagaattgttaaggaagaaatttttggaccagttgtcactgtcgcaaagttcaagactttagaagaaggtgtcgaaatggctaacagctctgaattcggtctaggttctggtatcgaaacagaatctttgagcacaggtttgaaggtggccaagatgttgaaggccggtaccgtctggatcaacacatacaacgattttgactccagagttccattcggtggtgttaagcaatctggttacggtagagaaatgggtgaagaagtctaccatgcatacactgaagtaaaagctgtcagaattaagttgtaa

SEQ ID NO: 30 - DNA, 2142 bp, Artificial Sequence
  Description of Artificial Sequence Synthetic ACS1 polynucleotide
atgtcgccctctgccgtacaatcatcaaaactagaagaacagtcaagtgaaattgacaagttgaaagcaaaaatgtcccagtctgccgccactgcgcagcagaagaaggaacatgagtatgaacatttgacttcggtcaagatcgtgccacaacggcccatctcagatagactgcagcccgcaattgctacccactattctccacacttggacgggttgcaggactatcagcgcttgcacaaggagtctattgaagaccctgctaagttcttcggttctaaagctacccaatttttaaactggtctaagccattcgataaggtgttcatcccagaccctaaaacgggcaggccctccttccagaacaatgcatggttcctcaacggccaattaaacgcctgttacaactgtgttgacagacatgccttgaagactcctaacaagaaagccattattttcgaaggtgacgagcctggccaaggctattccattacctacaaggaactacttgaagaagtttgtcaagtggcacaagtgctgacttactctatgggcgttcgcaagggcgatactgttgccgtgtacatgcctatggtcccagaagcaatcataaccttgttggccatttcccgtatcggtgccattcactccgtagtctttgccgggttttcttccaactccttgagagatcgtatcaacgatggggactctaaagttgtcatcactacagatgaatccaacagaggtggtaaagtcattgagactaaaagaattgttgatgacgcgctaagagagaccccaggcgtgagacacgtcttggtttatagaaagaccaacaatccatctgttgctttccatgcccccagagatttggattgggcaacagaaaagaagaaatacaagacctactatccatgcacacccgttgattctgaggatccattattcttgttgtatacgtctggttctactggtgcccccaagggtgttcaacattctaccgcaggttacttgctgggagctttgttgaccatgcgctacacttttgacactcaccaagaagacgttttcttcacagctggagacattggctggattacaggccacacttatgtggtttatggtcccttactatatggttgtgccactttggtctttgaagggactcctgcgtacccaaattactcccgttattgggatattattgatgaacacaaagtcacccaattttatgttgcgccaactgctttgcgtttgttgaaaagagctggtgattcctacatcgaaaatcattccttaaaatctttgcgttgcttgggttcggtcggtgagccaattgctgctgaagtttgggagtggtactctgaaaaaataggtaaaaatgaaatccccattgtagacacctactggcaaacagaatctggttcgcatctggtcaccccgctggctggtggtgttacaccaatgaaaccgggttctgcctcattccccttcttcggtattgatgcagttgttcttgaccctaacactggtgaagaacttaacaccagccacgcagagggtgtccttgccgtcaaagctgcatggccatcatttgcaagaactatttggaaaaatcatgataggtatctagacacttatttgaacccttaccctggctactatttcactggtgatggtgctgcaaaggataaggatggttatatctggattttgggtcgtgtagacgatgtggtgaacgtctctggtcaccgtctgtctaccgctgaaattgaggctgctattatcgaagatccaattgtggccgagtgtgctgttgtcggattcaacgatgacttgactggtcaagcagttgctgcatttgtggtgttgaaaaacaaatctagttggtccaccgcaacagatgatgaattacaagatatcaagaagcatttggtctttactgttagaaaagacatcgggccatttgccgcaccaaaattgatcattttagtggatgacttgcccaagacaagatccggcaaaattatgagacgtattttaagaaaaatcctagcaggagaaagtgaccaactaggcgacgtttctacattgtcaaaccctggcattgttagacatctaattgattcggtcaagttgtaa

SEQ ID NO: 31 - DNA, 2052 bp, Artificial Sequence
  Description of Artificial Sequence Synthetic ACS2 polynucleotide
atgacaatcaaggaacataaagtagtttatgaagctcacaacgtaaaggctcttaaggctcctcaacatttttacaacagccaacccggcaagggttacgttactgatatgcaacattatcaagaaatgtatcaacaatctatcaatgagccagaaaaattctttgataagatggctaaggaatacttgcattgggatgctccatacaccaaagttcaatctggttcattgaacaatggtgatgttgcatggtttttgaacggtaaattgaatgcatcatacaattgtgttgacagacatgcctttgctaatcccgacaagccagctttgatctatgaagctgatgacgaatccgacaacaaaatcatcacatttggtgaattactcagaaaagtttcccaaatcgctggtgtcttaaaaagctggggcgttaagaaaggtgacacagtggctatctatttgccaatgattccagaagcggtcattgctatgttggctgtggctcgtattggtgctattcactctgttgtctttgctgggttctccgctggttcgttgaaagatcgtgtcgttgacgctaattctaaagtggtcatcacttgtgatgaaggtaaaagaggtggtaagaccatcaacactaaaaaaattgttgacgaaggtttgaacggagtcgatttggtttcccgtatcttggttttccaaagaactggtactgaaggtattccaatgaaggccggtagagattactggtggcatgaggaggccgctaagcagagaacttacctacctcctgtttcatgtgacgctgaagatcctctatttttattatacacttccggttccactggttctccaaagggtgtcgttcacactacaggtggttatttattaggtgccgctttaacaactagatacgtttttgatattcacccagaagatgttctcttcactgccggtgacgtcggctggatcacgggtcacacctatgctctatatggtccattaaccttgggtaccgcctcaataattttcgaatccactcctgcctacccagattatggtagatattggagaattatccaacgtcacaaggctacccatttctatgtggctccaactgctttaagattaatcaaacgtgtaggtgaagccgaaattgccaaatatgacacttcctcattacgtgtcttgggttccgtcggtgaaccaatctctccagacttatgggaatggtatcatgaaaaagtgggtaacaaaaactgtgtcatttgtgacactatgtggcaaacagagtctggttctcatttaattgctcctttggcaggtgctgtcccaacaaaacctggttctgctaccgtgccattctttggtattaacgcttgtatcattgaccctgttacaggtgtggaattagaaggtaatgatgtcgaaggtgtccttgccgttaaatcaccatggccatcaatggctagatctgtttggaaccaccacgaccgttacatggatacttacttgaaaccttatcctggtcactatttcacaggtgatggtgctggtagagatcatgatggttactactggatcaggggtagagttgacgacgttgtaaatgtttccggtcatagattatccacatcagaaattgaagcatctatctcaaatcacgaaaacgtctcggaagctgctgttgtcggtattccagatgaattgaccggtcaaaccgtcgttgcatatgtttccctaaaagatggttatctacaaaacaacgctactgaaggtgatgcagaacacatcacaccagataatttacgtagagaattgatcttacaagttaggggtgagattggtcctttcgcctcaccaaaaaccattattctagttagagatctaccaagaacaaggtcaggaaagattatgagaagagttctaagaaaggttgcttctaacgaagccgaacagctaggtgacctaactactttggccaacccagaagttgtacctgccatcatttctgctgtagagaaccaatttttctctcaaaaaaagaaataa

SEQ ID NO: 32 - DNA, 5206 bp, Artificial Sequence
  Description of Artificial Sequence Synthetic pGV1428 polynucleotide
ccataacacagtcctttcccgcaattttctttttctattactcttggcctcctctagtacactctatatttttttatgcctcggtaatgattttcatttttttttttcccctagcggatgactctttttttttcttagcgattggcattatcacataatgaattatacattatataaagtaatgtgatttcttcgaagaatatactaaaaaatgagcaggcaagataaacgaaggcaaagatgacagagcagaaagccctagtaaagcgtattacaaatgaaaccaagattcagattgcgatctctttaaagggtggtcccctagcgatagagcactcgatcttcccagaaaaagaggcagaagcagtagcagaacaggccacacaatcgcaagtgattaacgtccacacaggtatagggtttctggaccatatgatacatgctctggccaagcattccggctggtcgctaatcgttgagtgcattggtgacttacacatagacgaccatcacaccactgaagactgcgggattgctctcggtcaagcttttaaagaggccctaggggccgtgcgtggagtaaaaaggtttggatcaggatttgcgcctttggatgaggcactttccagagcggtggtagatctttcgaacaggccgtacgcagttgtcgaacttggtttgcaaagggagaaagtaggagatctctcttgcgagatgatcccgcattttcttgaaagctttgcagaggctagcagaattaccctccacgttgattgtctgcgaggcaagaatgatcatcaccgtagtgagagtgcgttcaaggctcttgcggttgccataagagaagccacctcgcccaatggtaccaacgatgttccctccaccaaaggtgttcttatgtagtgacaccgattatttaaagctgcagcatacgatatatatacatgtgtatatatgtatacctatgaatgtcagtaagtatgtatacgaacagtatgatactgaagatgacaaggtaatgcatcattctatacgtgtcattctgaacgaggcgacgtcgccggcgatcacagcggacggtggtggcatgatggggcttgcgatgctatgtttgtttgttttgtgatgatgtatattattattgaaaaacgatatcagacatttgtctgataatgcttcattatcagacaaatgtctgatatcgtttggagaaaaagaaaaggaaaacaaactaaatatctactatataccactgtattttatactaatgactttctacgcctagtgtcaccctctcgtgtacccattgaccctgtatcggcgcgttgcctcgcgttcctgtaccatatatttttgtttatttaggtattaaaatttactttcctcatacaaatattaaattcaccaaacttctcaaaaactaattattcgtagttacaaactctattttacaatcacgtttattcaaccattctacatccaataaccaaaatgcccatgtacctctcagcgaagtccaacggtactgtccaatattctcattaaatagtctttcatctatatatcagaaggtaattataattagagatttcgaatcattaccgtgccgattcgcacgctgcaacggcatgcatcactaatgaaaagcatacgacgcctgcgtctgacatgcactcattctgaagaagattctgggcgcgtttcgttctcgttttcctctgtatattgtactctggtggacaatttgaacataacgtctttcacctcgccattctcaataatgggttccaattctatccaggtagcggttaattgacggtgcttaagccgtatgctcactctaacgctaccgttgtccaaacaacggacccctttgtgacgggtgtaagacccatcatgaagtaaaacatctctaacggtatggaaaagagtggtacggtcaagtttcctggcacgagtcaattttccctcttcgtgtagatcggtaccggccgcaaattaaagccttcgagcgtcccaaaaccttctcaagcaaggttttcagtataatgttacatgcgtacacgcgtctgtacagaaaaaaaagaaaaatttgaaatataaataacgttcttaatactaacataactataaaaaaataaatagggacctagacttcaggttgtctaactccttccttttcggttagagcggatgtggggggagggcgtgaatgtaagcgtgacataactaattacatgactcgagcggccgcggatcccgggaattcgtcgacaccatcttcttctgagatgagtttttgttccatgctagttctagaatccgtcgaaactaagttctggtgttttaaaactaaaaaaaagactaactataaaagtagaatttaagaagtttaagaaatagatttacagaattacaatcaatacctaccgtctttatatacttattagtcaagtaggggaataatttcagggaactggtttcaaccttttttttcagctttttccaaatcagagagagcagaaggtaatagaaggtgtaagaaaatgagatagatacatgcgtgggtcaattgccttgtgtcatcatttactccaggcaggttgcatcactccattgaggttgtgcccgttttttgcctgtttgtgcccctgttctctgtagttgcgctaagagaatggacctatgaactgatggttggtgaagaaaacaatattttggtgctgggattctttttttttctggatgccagcttaaaaagcgggctccattatatttagtggatgccaggaataaactgttcacccagacacctacgatgttatatattctgtgtaacccgccccctattttgggcatgtacgggttacagcagaattaaaaggctaattttttgactaaataaagttaggaaaatcactactattaattatttacgtattctttgaaatggcgagtattgataatgataaactgagctagatctgggcccgagctccagcttttgttccctttagtgagggttaattgcgcgcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgt

SEQ ID NO: 33 - DNA, 5157 bp, Artificial Sequence
  Description of Artificial Sequence Synthetic pGV1429 polynucleotide
caggcaagtgcacaaacaatacttaaataaatactactcagtaataacctatttcttagcatttttgacgaaatttgctattttgttagagtcttttacaccatttgtctccacacctccgcttacatcaacaccaataacgccatttaatctaagcgcatcaccaacattttctggcgtcagtccaccagctaacataaaatgtaagctttcggggctctcttgccttccaacccagtcagaaatcgagttccaatccaaaagttcacctgtcccacctgcttctgaatcaaacaagggaataaacgaatgaggtttctgtgaagctgcactgagtagtatgttgcagtcttttggaaatacgagtcttttaataactggcaaaccgaggaactcttggtattcttgccacgactcatctccatgcagttggacgatatcaatgccgtaatcattgaccagagccaaaacatcctccttaggttgattacgaaacacgccaaccaagtatttcggagtgcctgaactatttttatatgcttttacaagacttgaaattttccttgcaataaccgggtcaattgttctctttctattgggcacacatataatacccagcaagtcagcatcggaatctagagcacattctgcggcctctgtgctctgcaagccgcaaactttcaccaatggaccagaactacctgtgaaattaataacagacatactccaagctgcctttgtgtgcttaatcacgtatactcacgtgctcaatagtcaccaatgccctccctcttggccctctccttttcttttttcgaccgaattaattcttaatcggcaaaaaaagaaaagctccggatcaagattgtacgtaaggtgacaagctatttttcaataaagaatatcttccactactgccatctggcgtcataactgcaaagtacacatatattacgatgctgtctattaaatgcttcctatattatatatatagtaatgtcgttgacgtcgccggcgatcacagcggacggtggtggcatgatggggcttgcgatgctatgtttgtttgttttgtgatgatgtatattattattgaaaaacgatatcagacatttgtctgataatgcttcattatcagacaaatgtctgatatcgtttggagaaaaagaaaaggaaaacaaactaaatatctactatataccactgtattttatactaatgactttctacgcctagtgtcaccctctcgtgtacccattgaccctgtatcggcgcgttgcctcgcgttcctgtaccatatatttttgtttatttaggtattaaaatttactttcctcatacaaatattaaattcaccaaacttctcaaaaactaattattcgtagttacaaactctattttacaatcacgtttattcaaccattctacatccaataaccaaaatgcccatgtacctctcagcgaagtccaacggtactgtccaatattctcattaaatagtctttcatctatatatcagaaggtaattataattagagatttcgaatcattaccgtgccgattcgcacgctgcaacggcatgcatcactaatgaaaagcatacgacgcctgcgtctgacatgcactcattctgaagaagattctgggcgcgtttcgttctcgttttcctctgtatattgtactctggtggacaatttgaacataacgtctttcacctcgccattctcaataatgggttccaattctatccaggtagcggttaattgacggtgcttaagccgtatgctcactctaacgctaccgttgtccaaacaacggacccctttgtgacgggtgtaagacccatcatgaagtaaaacatctctaacggtatggaaaagagtggtacggtcaagtttcctggcacgagtcaattttccctcttcgtgtagatcggtaccggccgcaaattaaagccttcgagcgtcccaaaaccttctcaagcaaggttttcagtataatgttacatgcgtacacgcgtctgtacagaaaaaaaagaaaaatttgaaatataaataacgttcttaatactaacataactataaaaaaataaatagggacctagacttcaggttgtctaactccttccttttcggttagagcggatgtggggggagggcgtgaatgtaagcgtgacataactaattacatgactcgagcggccgcggatcccgggaattcgtcgacaccatcttcttctgagatgagtttttgttccatgctagttctagaatccgtcgaaactaagttctggtgttttaaaactaaaaaaaagactaactataaaagtagaatttaagaagtttaagaaatagatttacagaattacaatcaatacctaccgtctttatatacttattagtcaagtaggggaataatttcagggaactggtttcaaccttttttttcagctttttccaaatcagagagagcagaaggtaatagaaggtgtaagaaaatgagatagatacatgcgtgggtcaattgccttgtgtcatcatttactccaggcaggttgcatcactccattgaggttgtgcccgttttttgcctgtttgtgcccctgttctctgtagttgcgctaagagaatggacctatgaactgatggttggtgaagaaaacaatattttggtgctgggattctttttttttctggatgccagcttaaaaagcgggctccattatatttagtggatgccaggaataaactgttcacccagacacctacgatgttatatattctgtgtaacccgccccctattttgggcatgtacgggttacagcagaattaaaaggctaattttttgactaaataaagttaggaaaatcactactattaattatttacgtattctttgaaatggcgagtattgataatgataaactgagctagatctgggcccgagctccagcttttgttccctttagtgagggttaattgcgcgcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgt

SEQ ID NO: 34 - DNA, 6041 bp, Artificial Sequence
  Description of Artificial Sequence Synthetic pGV1430 polynucleotide
ccagttaactgtgggaatactcaggtatcgtaagatgcaagagttcgaatctcttagcaaccattatttttttcctcaacataacgagaacacacaggggcgctatcgcacagaatcaaattcgatgactggaaattttttgttaatttcagaggtcgcctgacgcatatacctttttcaactgaaaaattgggagaaaaaggaaaggtgagagcgccggaaccggcttttcatatagaatagagaagcgttcatgactaaatgcttgcatcacaatacttgaagttgacaatattatttaaggacctattgttttttccaataggtggttagcaatcgtcttactttctaacttttcttaccttttacatttcagcaatatatatatatatatttcaaggatataccattctaatgtctgcccctaagaagatcgtcgttttgccaggtgaccacgttggtcaagaaatcacagccgaagccattaaggttcttaaagctatttctgatgttcgttccaatgtcaagttcgatttcgaaaatcatttaattggtggtgctgctatcgatgctacaggtgttccacttccagatgaggcgctggaagcctccaagaaggctgatgccgttttgttaggtgctgtgggtggtcctaaatggggtaccggtagtgttagacctgaacaaggtttactaaaaatccgtaaagaacttcaattgtacgccaacttaagaccatgtaactttgcatccgactctcttttagacttatctccaatcaagccacaatttgctaaaggtactgacttcgttgttgtcagagaattagtgggaggtatttactttggtaagagaaaggaagacgatggtgatggtgtcgcttgggatagtgaacaatacaccgttccagaagtgcaaagaatcacaagaatggccgctttcatggccctacaacatgagccaccattgcctatttggtccttggataaagctaatgttttggcctcttcaagattatggagaaaaactgtggaggaaaccatcaagaacgaattccctacattgaaggttcaacatcaattgattgattctgccgccatgatcctagttaagaacccaacccacctaaatggtattataatcaccagcaacatgtttggtgatatcatctccgatgaagcctccgttatcccaggttccttgggtttgttgccatctgcgtccttggcctctttgccagacaagaacaccgcatttggtttgtacgaaccatgccacggttctgctccagatttgccaaagaataaggtcaaccctatcgccactatcttgtctgctgcaatgatgttgaaattgtcattgaacttgcctgaagaaggtaaggccattgaagatgcagttaaaaaggttttggatgcaggtatcagaactggtgatttaggtggttccaacagtaccaccgaagtcggtgatgctgtcgccgaagaagttaagaaaatccttgcttaaaaagattctctttttttatgatatttgtacataaactttataaatgaaattcataatagaaacgacacgaaattacaaaatggaatatgttcatagggtagacgaaactatatacgcaatctacatacatttatcaagaaggagaaaaaggaggatgtaaaggaatacaggtaagcaaattgatactaatggctcaacgtgataaggaaaaagaattgcactttaacattaatattgacaaggaggagggcaccacacaaaaagttaggtgtaacagaaaatcatgaaactatgattcctaatttatatattggaggattttctctaaaaaaaaaaaaatacaacaaataaaaaacactcaatgacctgaccatttgatggagttgccggcgatcacagcggacggtggtggcatgatggggcttgcgatgctatgtttgtttgttttgtgatgatgtatattattattgaaaaacgatatcagacatttgtctgataatgcttcattatcagacaaatgtctgatatcgtttggagaaaaagaaaaggaaaacaaactaaatatctactatataccactgtattttatactaatgactttctacgcctagtgtcaccctctcgtgtacccattgaccctgtatcggcgcgttgcctcgcgttcctgtaccatatatttttgtttatttaggtattaaaatttactttcctcatacaaatattaaattcaccaaacttctcaaaaactaattattcgtagttacaaactctattttacaatcacgtttattcaaccattctacatccaataaccaaaatgcccatgtacctctcagcgaagtccaacggtactgtccaatattctcattaaatagtctttcatctatatatcagaaggtaattataattagagatttcgaatcattaccgtgccgattcgcacgctgcaacggcatgcatcactaatgaaaagcatacgacgcctgcgtctgacatgcactcattctgaagaagattctgggcgcgtttcgttctcgttttcctctgtatattgtactctggtggacaatttgaacataacgtctttcacctcgccattctcaataatgggttccaattctatccaggtagcggttaattgacggtgcttaagccgtatgctcactctaacgctaccgttgtccaaacaacggacccctttgtgacgggtgtaagacccatcatgaagtaaaacatctctaacggtatggaaaagagtggtacggtcaagtttcctggcacgagtcaattttccctcttcgtgtagatcggtaccggccgcaaattaaagccttcgagcgtcccaaaaccttctcaagcaaggttttcagtataatgttacatgcgtacacgcgtctgtacagaaaaaaaagaaaaatttgaaatataaataacgttcttaatactaacataactataaaaaaataaatagggacctagacttcaggttgtctaactccttccttttcggttagagcggatgtggggggagggcgtgaatgtaagcgtgacataactaattacatgactcgagcggccgcggatcccgggaattcgtcgacaccatcttcttctgagatgagtttttgttccatgctagttctagaatccgtcgaaactaagttctggtgttttaaaactaaaaaaaagactaactataaaagtagaatttaagaagtttaagaaatagatttacagaattacaatcaatacctaccgtctttatatacttattagtcaagtaggggaataatttcagggaactggtttcaaccttttttttcagctttttccaaatcagagagagcagaaggtaatagaaggtgtaagaaaatgagatagatacatgcgtgggtcaattgccttgtgtcatcatttactccaggcaggttgcatcactccattgaggttgtgcccgttttttgcctgtttgtgcccctgttctctgtagttgcgctaagagaatggacctatgaactgatggttggtgaagaaaacaatattttggtgctgggattctttttttttctggatgccagcttaaaaagcgggctccattatatttagtggatgccaggaataaactgttcacccagacacctacgatgttatatattctgtgtaacccgccccctattttgggcatgtacgggttacagcagaattaaaaggctaattttttgactaaataaagttaggaaaatcactactattaattatttacgtattctttgaaatggcgagtattgataatgataaactgagctagatctgggcccgagctccagcttttgttccctttagtgagggttaattgcgcgcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgt

SEQ ID NO: 35 - DNA, 5639 bp, Artificial Sequence
  Description of Artificial Sequence Synthetic pGV1431 polynucleotide
ctgattggaaagaccattctgctttacttttagagcatcttggtcttctgagctcattatacctcaatcaaaactgaaattaggtgcctgtcacggctctttttttactgtacctgtgacttcctttcttatttccaaggatgctcatcacaatacgcttctagatctattatgcattataattaatagttgtagctacaaaaggtaaaagaaagtccggggcaggcaacaatagaaatcggcaaaaaaaactacagaaatactaagagcttcttccccattcagtcatcgcatttcgaaacaagaggggaatggctctggctagggaactaaccaccatcgcctgactctatgcactaaccacgtgactacatatatgtgatcgtttttaacatttttcaaaggctgtgtgtctggctgtttccattaattttcactgattaagcagtcatattgaatctgagctcatcaccaacaagaaatactaccgtaaaagtgtaaaagttcgtttaaatcatttgtaaactggaacagcaagaggaagtatcatcagctagccccataaactaatcaaaggaggatgtctactaagagttactcggaaagagcagctgctcatagaagtccagttgctgccaagcttttaaacttgatggaagagaagaagtcaaacttatgtgcttctcttgatgttcgtaaaacagcagagttgttaagattagttgaggttttgggtccatatatctgtctattgaagacacatgtagatatcttggaggatttcagctttgagaataccattgtgccgttgaagcaattagcagagaaacacaagtttttgatatttgaagacaggaagtttgccgacattgggaacactgttaaattacaatacacgtctggtgtataccgtatcgccgaatggtctgatatcaccaatgcacacggtgtgactggtgcgggcattgttgctggtttgaagcaaggtgccgaggaagttacgaaagaacctagagggttgttaatgcttgccgagttatcgtccaaggggtctctagcgcacggtgaatacactcgtgggaccgtggaaattgccaagagtgataaggactttgttattggatttattgctcaaaacgatatgggtggaagagaagagggctacgattggttgatcatgacgccaggtgttggtcttgatgacaaaggtgatgctttgggacaacaatacagaactgtggatgaagttgttgccggtggatcagacatcattattgttggtagaggtcttttcgcaaagggaagagatcctgtagtggaaggtgagagatacagaaaggcgggatgggacgcttacttgaagagagtaggcagatccgcttaagagttctccgagaacaagcagaggttcgagtgtactcggatcagaagttacaagttgatcgtttatatataaactatacagagatgttagagtgtaatggcattgcgtgccggcgatcacagcggacggtggtggcatgatggggcttgcgatgctatgtttgtttgttttgtgatgatgtatattattattgaaaaacgatatcagacatttgtctgataatgcttcattatcagacaaatgtctgatatcgtttggagaaaaagaaaaggaaaacaaactaaatatctactatataccactgtattttatactaatgactttctacgcctagtgtcaccctctcgtgtacccattgaccctgtatcggcgcgttgcctcgcgttcctgtaccatatatttttgtttatttaggtattaaaatttactttcctcatacaaatattaaattcaccaaacttctcaaaaactaattattcgtagttacaaactctattttacaatcacgtttattcaaccattctacatccaataaccaaaatgcccatgtacctctcagcgaagtccaacggtactgtccaatattctcattaaatagtctttcatctatatatcagaaggtaattataattagagatttcgaatcattaccgtgccgattcgcacgctgcaacggcatgcatcactaatgaaaagcatacgacgcctgcgtctgacatgcactcattctgaagaagattctgggcgcgtttcgttctcgttttcctctgtatattgtactctggtggacaatttgaacataacgtctttcacctcgccattctcaataatgggttccaattctatccaggtagcggttaattgacggtgcttaagccgtatgctcactctaacgctaccgttgtccaaacaacggacccctttgtgacgggtgtaagacccatcatgaagtaaaacatctctaacggtatggaaaagagtggtacggtcaagtttcctggcacgagtcaattttccctcttcgtgtagatcggtaccggccgcaaattaaagccttcgagcgtcccaaaaccttctcaagcaaggttttcagtataatgttacatgcgtacacgcgtctgtacagaaaaaaaagaaaaatttgaaatataaataacgttcttaatactaacataactataaaaaaataaatagggacctagacttcaggttgtctaactccttccttttcggttagagcggatgtggggggagggcgtgaatgtaagcgtgacataactaattacatgactcgagcggccgcggatcccgggaattcgtcgacaccatcttcttctgagatgagtttttgttccatgctagttctagaatccgtcgaaactaagttctggtgttttaaaactaaaaaaaagactaactataaaagtagaatttaagaagtttaagaaatagatttacagaattacaatcaatacctaccgtctttatatacttattagtcaagtaggggaataatttcagggaactggtttcaaccttttttttcagctttttccaaatcagagagagcagaaggtaatagaaggtgtaagaaaatgagatagatacatgcgtgggtcaattgccttgtgtcatcatttactccaggcaggttgcatcactccattgaggttgtgcccgttttttgcctgtttgtgcccctgttctctgtagttgcgctaagagaatggacctatgaactgatggttggtgaagaaaacaatattttggtgctgggattctttttttttctggatgccagcttaaaaagcgggctccattatatttagtggatgccaggaataaactgttcacccagacacctacgatgttatatattctgtgtaacccgccccctattttgggcatgtacgggttacagcagaattaaaaggctaattttttgactaaataaagttaggaaaatcactactattaattatttacgtattctttgaaatggcgagtattgataatgataaactgagctagatctgggcccgagctccagcttttgttccctttagtgagggttaattgcgcgcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgt

SEQ ID NO: 36 - DNA, 741 bp, Artificial Sequence
  Description of Artificial Sequence Synthetic pflA polynucleotide
atgtcagttattggtcgcattcactcctttgaatcctgtggaaccgtagacggcccaggtattcgctttatcacctttttccagggctgcctgatgcgctgcctgtattgtcataaccgcgacacctgggacacgcatggcggtaaagaagttaccgttgaagatttgatgaaggaagtggtgacctatcgccactttatgaacgcttccggcggcggcgttaccgcatccggcggtgaagcaatcctgcaagctgagtttgttcgtgactggttccgcgcctgcaaaaaagaaggcattcatacctgtctggacaccaacggttttgttcgtcgttacgatccggtgattgatgaactgctggaagtaaccgacctggtaatgctcgatctcaaacagatgaacgacgagatccaccaaaatctggttggagtttccaaccaccgcacgctggagttcgctaaatatctggcgaacaaaaatgtgaaggtgtggatccgctacgttgttgtcccaggctggtctgacgatgacgattcagcgcatcgcctcggtgaatttacccgtgatatgggcaacgttgagaaaatcgagcttctcccctaccacgagctgggcaaacacaaatgggtggcaatgggtgaagagtacaaactcgacggtgttaaaccaccgaagaaagagaccatggaacgcgtgaaaggcattcttgagcagtacggtcataaggtaatgttctaa

SEQ ID NO: 37 - DNA, 2283 bp, Artificial Sequence
  Description of Artificial Sequence Synthetic pflB polynucleotide
atgtccgagcttaatgaaaagttagccacagcctgggaaggttttaccaaaggtgactggcagaatgaagtaaacgtccgtgacttcattcagaaaaactacactccgtacgagggtgacgagtccttcctggctggcgctactgaagcgaccaccaccctgtgggacaaagtaatggaaggcgttaaactggaaaaccgcactcacgcgccagttgactttgacaccgctgttgcttccaccatcacctctcacgacgctggctacatcaacaagcagcttgagaaaatcgttggtctgcagactgaagctccgctgaaacgtgctcttatcccgttcggtggtatcaaaatgatcgaaggttcctgcaaagcgtacaaccgcgaactggatccgatgatcaaaaaaatcttcactgaataccgtaaaactcacaaccagggcgtgttcgacgtttacactccggacatcctgcgttgccgtaaatctggtgttctgaccggtctgccagatgcatatggccgtggccgtatcatcggtgactaccgtcgcgttgcgctgtacggtatcgactacctgatgaaagacaaactggcacagttcacttctctgcaggctgatctggaaaacggcgtaaacctggaacagactatccgtctgcgcgaagaaatcgctgaacagcaccgcgctctgggtcagatgaaagaaatggctgcgaaatacggctacgacatctctggtccggctaccaacgctcaggaagctatccagtggacttacttcggctacctggctgctgttaagtctcagaacggtgctgcaatgtccttcggtcgtacctccaccttcctggatgtgtacatcgaacgtgacctgaaagctggcaagatcaccgaacaagaagcgcaggaaatggttgaccacctggtcatgaaactgcgtatggttcgcttcctgcgtactccggaatacgatgaactgttctctggcgacccgatctgggcaaccgaatctatcggtggtatgggcctcgacggtcgtaccctggttaccaaaaacagcttccgtttcctgaacaccctgtacaccatgggtccgtctccggaaccgaacatgaccattctgtggtctgaaaaactgccgctgaacttcaagaaattcgccgctaaagtgtccatcgacacctcttctctgcagtatgagaacgatgacctgatgcgtccggacttcaacaacgatgactacgctattgcttgctgcgtaagcccgatgatcgttggtaaacaaatgcagttcttcggtgcgcgtgcaaacctggcgaaaaccatgctgtacgcaatcaacggcggcgttgacgaaaaactgaaaatgcaggttggtccgaagtctgaaccgatcaaaggcgatgtcctgaactatgatgaagtgatggagcgcatggatcacttcatggactggctggctaaacagtacatcactgcactgaacatcatccactacatgcacgacaagtacagctacgaagcctctctgatggcgctgcacgaccgtgacgttatccgcaccatggcgtgtggtatcgctggtctgtccgttgctgctgactccctgtctgcaatcaaatatgcgaaagttaaaccgattcgtgacgaagacggtctggctatcgacttcgaaatcgaaggcgaatacccgcagtttggtaacaatgatccgcgtgtagatgacctggctgttgacctggtagaacgtttcatgaagaaaattcagaaactgcacacctaccgtgacgctatcccgactcagtctgttctgaccatcacttctaacgttgtgtatggtaagaaaacgggtaacaccccagacggtcgtcgtgctggcgcgccgttcggaccgggtgctaacccgatgcacggtcgtgaccagaaaggtgcagtagcctctctgacttccgttgctaaactgccgtttgcttacgctaaagatggtatctcctacaccttctctatcgttccgaacgcactgggtaaagacgacgaagttcgtaagaccaacctggctggtctgatggatggttacttccaccacgaagcatccatcgaaggtggtcagcacctgaacgttaacgtgatgaaccgtgaaatgctgctcgacgcgatggaaaacccggaaaaatatccgcagctgaccatccgtgtatctggctacgcagtacgtttcaactcgctgactaaagaacagcagcaggacgttattactcgtaccttcactcaatctatgtaa

SEQ ID NO: 38 - DNA, 1095 bp, Artificial Sequence
  Description of Artificial Sequence Synthetic Cb-FDH1 polynucleotide
atgaagatcgttttagtcttatatggtgctggtaaacacgctgccgatgaagaaaaattatacggttgtactgaaaacaaattaggtattgccaattggttgaaagatcaaggacatgaactaatcaccacgtctgataaagaaggcggaaacagtgtgttggatcaacatataccagatgccgatattatcattacaactcctttccatcctgcttatatcactaaggaaagaatcgacaaggctaaaaaattgaaattagttgttgtcgctggtgtcggttctgatcatattgatttggattatatcaaccaaacaggtaggaaaatctccgtcttggaagttaccggttctaatgttgtctctgttgcagaacacgttgtcatgaccatgcttgtcttggttagaaattttgttccagctcacgaacaaaacattaaccacgattgggaggttgctgctatcgctaaggatgcttacgatatcgaaggtaaaactatcgccaccattggtgccggtagaattggttacagagtcttggaaagattagtcccattcaatcctaaagaattattatactacgattatcaagctttaccaaaagatgctgaagaaaaagttggtgctagaagggttgaaaatattgaagaattggttgcccaagctgatatagttacagttaatgctccattacacgctggtacaaaaggtttaattaacaaggaattattgtctaaattcaagaaaggtgcttggttagtcaatactgcaagaggtgccatttgtgttgccgaagatgttgctgcagctttagaatctggtcaattaagaggttatggtggtgatgtttggttcccacaaccagctccaaaagatcacccatggagagatatgagaaacaaatatggtgctggtaacgccacgactcctcattactctggtactactttagatgctcaaactagatacgctcaaggtactaaaaatatcttggagtcattctttactggtaagtttgattacagaccacaagatatcatcttattaaacggtgaatacgttaccaaagcttacggtaaacacgataagaaataa

SEQ ID NO: 39 - DNA, 1524 bp, Artificial Sequence
  Description of Artificial Sequence Synthetic KlALD6 polynucleotide
atgtcctctacaattgctgagaaattgaacctcaagatcgtcgaacaagacgctgttagcatcactttgccaaacggtttgacttaccaacaaccaactggtttgttcatcaacaatcagttcatcaagtctcaagacggtaagactttgaaggttgaaaacccatctactgaggaaatcattgtcgaagtccaatctgctacttctcaagacgtcgagtacgccgttgaagctgccgatgctgctttcaactccgaatggtctactatggacccaaaaaagcgtggttctttgttgtttaagttggctgacttgattgaagctcaaaaggaattgattgcttctatcgaatctgctgacaacggtaagactttggccctagccagaggtgatgttggtttggtcattgactacatcagatctgctgctggttatgctgacaagttgggtggtagaactatcaacactggtgatggttacgctaacttcacttacaaggaacctctaggtgtctgtggtcaaatcatcccatggaacttcccattgatgatgctttcttggaagatcgcccctgctttggttgctggtaacaccgttatcttgaagccagcttccccaaccccattgaacgctttgttctttgcttctttgtgtaaggaagcaggtatcccagctggtgtcgttaacatcgttccaggtccaggtagatccgttggtgacaccatcaccaaccatccaaaaattagaaagattgccttcactggttccactgacattggtagagacgttgctatcaaggctgcccaatctaacttgaagaaggtcaccttggaattgggtggtaaatccgctcatttggtctttgaagatgccaacattaagaagactattccaaacttggtcaacggtattttcaagaatgctggtcaaatttgttcctctggttccagaatctatgtccaagacaccatctacgatcaactattgtctgaattcaagacttacctggaaactgaaattaaggtcggttccccattcgatgaatctaacttccaagctgctatcaacaacaaggctcaattcgaaactatcttgaactacatcgacatcggtaagaaggaaggtgcttctatcttgactggtggtgaaagagtaggcaacaagggttacttcattaaaccaactgtattctacaacgttaaggaagatatgagaatcgtcaaggaagaaatctttggtcctgtcgtcaccatctccaagttctctactgtcgacgaagctgtcgctttggctaacgactccgaattcggtttgggtgctggtatcgaaactgaaaacatctccgttgccttgaaggtcgcaaagagactaaaagctggtaccgtctggatcaacacttacaacgatttcgacgctgccgttccattcggtggttacaagcaatctggttacggtagagaaatgggtgaagaagctttcgaatcttacactcaaatcaaggccgtcaggatcaagttggattaa

SEQ ID NO: 40 - DNA, 2124 bp, Artificial Sequence
  Description of Artificial Sequence Synthetic KlACS1 polynucleotide
atgtctcctgctgttgataccgcttccaccgccaaagatccaatctcagtcatgaaatctaacgcttcagctgccgctgcagaccaaattaagacccatgaatacgaacatttaacttctgtgcctatagtgcagcctctaccaattactgataggttgagcagcgaagcagctcaaaaatataaacctaatttgccaggtgggttcgaagagtacaagtctttgcacaaggaatcacttgaaaatccagccaagttttaccatgaacgtgctcagctgttgaattggttcaaaccatacgatcaagttttcatcccagataccgaaggtaaaccaacttttgagaacaacgcttggtttaccaacggtcaattgaacgcttgttacaatttggtagacagacatgccttcactcaaccaaacaaggttgccattctttatgaagctgatgaaccaggtcaaggttatagtctcacttatgcggaattgttagaacaagtctgtaaagttgctcaaatcttgcaatactcgatgaacgtcaagaaaggtgacacggtcgcagtttatatgccaatgatcccacaggctttgattaccttgttggcaattactcgtatcggtgccattcattccgttgtttttgctgggttctcttcgaattcattgcgtgatcgtattaacgatgcttactcaaagacagtcatcaccaccgatgaatctaagagaggtggtaagaccatcgaaaccaagcgtatcgtcgatgaagccttgaaggatacccctcaagtaacaaacgttttggtcttcaaacgtactcataacgaaaatatcaagtacattccaggtagggatttggactgggatgaggaagtcaagaagtacaaatcttacaccccatgcgaacctgttgactctgaacatcctttgttcttattgtatacttcgggttccaccggtgctccaaagggtgttcaacattctacagcaggttacttgctccaagcattattaagtatgaaatacacctttgacatccaaaacgatgacatcttcttcaccgcaggtgacattggttggatcactggtcacacatactgtgtttacggtccattgttacaaggttgtactactttggtgttcgaaggtacacctgcctatccaaacttttctcgttattgggaaattgttgacaagtaccaagtgactcaattctatgtagccccaactgcactacgtctattgaagagagctggtgattcctttactgaaggattctctctcaagtcattgcgctccttgggttccgttggtgaacctatcgctgctgaagtttgggaatggtactctgaaaagattggtaagaatgagctaccaatcgtagacacatactggcaaactgaatctggctcccacttggtcactccattggctggtggtgctactccaatgaaaccaggtgcagcggcattcccattctttggtattgatttggcagtgttggatccaaccacaggtatcgagcaaactggtgaacatgcagaaggtgttcttgccattaaaagaccttggccatctttcgcaagaaccatttggaagaataacgataggttcttagacacgtacttgaaaccatacccgggctattacttcactggtgatggtgttgcccgtgataaagatggattcttctggatcttgggtcgtgttgatgatgttgttaacgtctcaggtcacaggttgtctactgctgaaattgaagctgctatcattgaagatgatatggttgccgaatgtgcagttgttgggtttaacgacgaattgactggtcaagccgttgctgcctttgtagtattgaagaacaagtctagtttaactgctgcaagcgagtccgagttacaagacatcaaaaagcatttgatcatcaccgttagaaaggatattggtccattcgctgctcctaagttgatcgtcctagttgatgatctaccaaagactagatctggcaagattatgagacgtattttgagaaagatcctagccggtgaatctgatcaattgggcgacgtctccacattatccaaccctggtatcgttaagcacttgatcgattccgtgaaattataa

SEQ ID NO: 41 - DNA, 2055 bp, Artificial Sequence
  Description of Artificial Sequence Synthetic KlACS2 polynucleotide
atgtcgtcggataaattgcataaggttgtgcatgaagctcacgatgttgaagctcgtcatgctccagaacatttctacaattctcaaccgggtaaatcgtactgtactgatgaagaacattaccgtgagatgtacactcagtccattgaggacccagcagggtttttcggtccattggccaaggaatatctagattgggatcgtccattcacccaagtccaaagcggttctttggaacacggtgacattgcctggttcttaaatggtgaactgaatgcttcttataactgtgttgacagacacgcttttgccaacccagacaagccagctttaatctacgaagccgacgatgaatctgaaaacaaggtgatcacttttggcgaattgttgagacaggtctctgaagtggctggtgtcttgcaatcttggggtgtcaagaaaggagacaccgtcgccgtttacttgccaatgattcctgctgcagttgttgctatgttggccgttgcaagattaggtgccattcattcggttatctttgccggtttctctgccggttccttgaaggaaagagttgtcgatgcaggctgtaaagtggtcatcacttgcgatgaaggtaagagaggtggtaagaccgttcataccaagaagattgtcgacgaaggtttggccggtgtcgattccgtttccaagatcttggttttccaaagaactggtactcaaggtatcccaatgaagccagctagagatttctggtggcacgaagagtgtgtcaagcaaagaggttacttgccacctgtcccagtcaactccgaagatccattgttcttgttgtacacctctggttccaccggttctccaaaaggtgtcgtgcactctactgctggttacttgttaggttctgctttgaccaccagattcgttttcgatattcatccagaagatgttttgttcactgctggtgacgtcggttggattaccggccacacttacgccttgtacggtccattgaccttaggtaccgctaccattattttcgaatctactccagcttacccagattatggtagatactggagaatcattgaacgtcatagagctacccacttctacgtcgccccaactgccctaagattgatcaaacgtgtcggtgaagaagaaattgccaagtatgatacctcctccttaagagtcttgggttctgtcggtgaaccaatctctccagatctatgggaatggtaccacgaaaaggttggtaagaataactgtgttatctgtgacaccatgtggcaaaccgaatccggttcacacttgattgccccattggctggtgctgtcccaaccaaaccaggttccgctaccgtcccattcttcggtattaacgcctgtatcatcgacccagtttctggtgaagaattgaagggtaacgatgttgaaggtgtcttggcagtgaagtccccatggccttctatggccagatctgtctggaacaaccatgctcgttacttcgaaacttatttgaagccatacccaggatactacttcacaggtgatggtgctggtagagatcacgacggttactactggatcagaggtagagttgacgatgtcgttaacgtttccggtcacagactttctactgctgaaatcgaagctgctctagctgaacacgaaggtgtttctgaagctgccgttgttggtatcactgatgaactaacaggtcaagctgtcattgcattcgtttccttgaaggacggctatctgtctgaaaatgcggtagagggtgacagtacccacatctctccagacaacttacgtcgtgagttgattctacaagtcagaggtgaaattggtccattcgctgcaccaaagaccgttgttgttgtcaacgatttgccaaaaactagatccggtaaaattatgagaagagtcttgagaaaggttgcatccaaggaagctgatcaattgggtgatctaagtaccttagccaatgcagacgttgtaccatctatcatttctgcagtagaaaatcaatttttcagtcagcagaagaaataa

SEQ ID NO: 42 - DNA, 37 bp, Artificial Sequence
  Description of Artificial Sequence Synthetic Gevo-311 primer
gaggttgtcgacatgaaaaagatttttgtacttggag

SEQ ID NO: 43 - DNA, 35 bp, Artificial Sequence
  Description of Artificial Sequence Synthetic Gevo-175 primer
aattggatccttatttagaataatcatagaatcct

SEQ ID NO: 44 - DNA, 37 bp, Artificial Sequence
  Description of Artificial Sequence Synthetic Gevo-312 primer
gttcttgtcgacatggaattaaaaaatgttattcttg

SEQ ID NO: 45 - DNA, 37 bp, Artificial Sequence
  Description of Artificial Sequence Synthetic Gevo-171 primer
aattggatccttatttattttgaaaattcttttctgc

SEQ ID NO: 46 - DNA, 37 bp, Artificial Sequence
  Description of Artificial Sequence Synthetic Gevo-313 primer
caagaggtcgacatgaatttccaattaactagagaac

SEQ ID NO: 47 - DNA, 34 bp, Artificial Sequence
  Description of Artificial Sequence Synthetic Gevo-314 primer
gcgtccggatccctatcttaaaatgcttcctgcg

SEQ ID NO: 48 - DNA, 36 bp, Artificial Sequence
  Description of Artificial Sequence Synthetic Gevo-315 primer
cggaaagtcgacatgaatatagcagattacaaaggc

SEQ ID NO: 49 - DNA, 35 bp, Artificial Sequence
  Description of Artificial Sequence Synthetic Gevo-173 primer
aattggatccttattcagcgctctttatttcttta

SEQ ID NO: 50 - DNA, 37 bp, Artificial Sequence
  Description of Artificial Sequence Synthetic Gevo-316 primer
caaaatgtcgacatgaatatagtagtttgtgtaaaac

SEQ ID NO: 51 - DNA, 37 bp, Artificial Sequence
  Description of Artificial Sequence Synthetic Gevo-317 primer
taatttggatccttagatgtagtgtttttcttttaat

SEQ ID NO: 52 - DNA, 35 bp, Artificial Sequence
  Description of Artificial Sequence Synthetic Gevo-319 primer
gaaccagtcgacatggcacgttttactttaccaag

SEQ ID NO: 53 - DNA, 35 bp, Artificial Sequence
  Description of Artificial Sequence Synthetic Gevo-177 primer
aattggatccttacaaattaactttagttccatag

SEQ ID NO: 54 - DNA, 36 bp, Artificial Sequence
  Description of Artificial Sequence Synthetic Gevo-318 primer
tccatagtcgacatgaataaagacacactaatacct

SEQ ID NO: 55 - DNA, 40 bp, Artificial Sequence
  Description of Artificial Sequence Synthetic Gevo-249 primer
aattggatccttagccggcaagtacacatcttctttgtct

SEQ ID NO: 56 - DNA, 35 bp, Artificial Sequence
  Description of Artificial Sequence Synthetic Gevo-308 primer
gatcgagtcgacatgaaagaagttgtaatagctag

SEQ ID NO: 57 - DNA, 35 bp, Artificial Sequence
  Description of Artificial Sequence Synthetic Gevo-309 primer
gttataggatccctagcacttttctagcaatattg

SEQ ID NO: 58 - DNA, 37 bp, Artificial Sequence
  Description of Artificial Sequence Synthetic Gevo-281 primer
gtggatgtcgacatgaaaaaggtatgtgttataggtg

SEQ ID NO: 59 - DNA, 35 bp, Artificial Sequence
  Description of Artificial Sequence Synthetic Gevo-161 primer
aattggatccttattttgaataatcgtagaaacct

SEQ ID NO: 60 - DNA, 35 bp, Artificial Sequence
  Description of Artificial Sequence Synthetic Gevo-282 primer
tcctacgtcgacatggaactaaacaatgtcatcct

SEQ ID NO: 61 - DNA, 36 bp, Artificial Sequence
  Description of Artificial Sequence Synthetic Gevo-283 primer
taacttggatccctatctatttttgaagccttcaat

SEQ ID NO: 62 - DNA, 37 bp, Artificial Sequence
  Description of Artificial Sequence Synthetic Gevo-284 primer
caagaggtcgacatggattttaatttaacaagagaac

SEQ ID NO: 63 - DNA, 39 bp, Artificial Sequence
  Description of Artificial Sequence Synthetic Gevo-285 primer
caataaggatccttatctaaaaatttttcctgaaataac

SEQ ID NO: 64 - DNA, 36 bp, Artificial Sequence
  Description of Artificial Sequence Synthetic Gevo-286 primer
cgggaagtcgacatgaataaagcagattacaagggc

SEQ ID NO: 65 - DNA, 36 bp, Artificial Sequence
  Description of Artificial Sequence Synthetic Gevo-287 primer
gttcaaggatccttaattattagcagctttaacttg

SEQ ID NO: 66 - DNA, 38 bp, Artificial Sequence
  Description of Artificial Sequence Synthetic Gevo-288 primer
caaaattgtcgacatgaatatagttgtttgtttaaaac

SEQ ID NO: 67 - DNA, 41 bp, Artificial Sequence
  Description of Artificial Sequence Synthetic Gevo-289 primer
gttttaggatccttaaatatagtgttcttcttttaattttg

SEQ ID NO: 68 - DNA, 37 bp, Artificial Sequence
  Description of Artificial Sequence Synthetic Gevo-292 primer
caagaagtcgacatgaaagttacaaatcaaaaagaac

SEQ ID NO: 69 - DNA, 40 bp, Artificial Sequence
  Description of Artificial Sequence Synthetic Gevo-293 primer
tcctatgcggccgcttaaaatgattttatatagatatcct

SEQ ID NO: 70 - DNA, 35 bp, Artificial Sequence
  Description of Artificial Sequence Synthetic Gevo-290 primer
aggaaagtcgacatgaaagtcacaacagtaaagga

SEQ ID NO: 71 - DNA, 40 bp, Artificial Sequence
  Description of Artificial Sequence Synthetic Gevo-291 primer
atttaagcggccgcttaaggttgttttttaaaacaattta

SEQ ID NO: 72 - DNA, 37 bp, Artificial Sequence
  Description of Artificial Sequence Synthetic Gevo-294 primer
cataacgtcgacatgctaagttttgattattcaatac

SEQ ID NO: 73 - DNA, 36 bp, Artificial Sequence
  Description of Artificial Sequence Synthetic Gevo-247 primer
aattggatccttaataagattttttaaatatctcaa

SEQ ID NO: 74 - DNA, 37 bp, Artificial Sequence
  Description of Artificial Sequence Synthetic Gevo-295 primer
cataacgtcgacatggttgatttcgaatattcaatac

SEQ ID NO: 75 - DNA, 36 bp, Artificial Sequence
  Description of Artificial Sequence Synthetic Gevo-159 primer
aattggatccttacacagattttttgaatatttgta

SEQ ID NO: 76 - DNA, 35 bp, Artificial Sequence
  Description of Artificial Sequence Synthetic Gevo-310 primer
gatcgagaattcatgaaagaagttgtaatagctag

SEQ ID NO: 77 - DNA, 35 bp, Artificial Sequence; identical to SEQ ID NO: 57
  Description of Artificial Sequence Synthetic Gevo-309 primer

SEQ ID NO: 78 - DNA, 36 bp, Artificial Sequence
  Description of Artificial Sequence Synthetic Gevo-296 primer
cggatagtcgacatgaaaaaggtatgtgttataggc

SEQ ID NO: 79 - DNA, 38 bp, Artificial Sequence
  Description of Artificial Sequence Synthetic Gevo-297 primer
tcccaaggatccttattttgaataatcgtagaaaccct

SEQ ID NO: 80 - DNA, 35 bp, Artificial Sequence; identical to SEQ ID NO: 60
  Description of Artificial Sequence Synthetic Gevo-282 primer

SEQ ID NO: 81 - DNA, 36 bp, Artificial Sequence; identical to SEQ ID NO: 61
  Description of Artificial Sequence Synthetic Gevo-283 primer

SEQ ID NO: 82 - DNA, 37 bp, Artificial Sequence; identical to SEQ ID NO: 62
  Description of Artificial Sequence Synthetic Gevo-284 primer

SEQ ID NO: 83 - DNA, 37 bp, Artificial Sequence
  Description of Artificial Sequence Synthetic Gevo-298 primer
gtaaagggatccttaactaaaaatttttcctgaaatg

SEQ ID NO: 84 - DNA, 36 bp, Artificial Sequence; identical to SEQ ID NO: 64
  Description of Artificial Sequence Synthetic Gevo-286 primer

SEQ ID NO: 85 - DNA, 36 bp, Artificial Sequence
  Description of Artificial Sequence Synthetic Gevo-299 primer
gttcaaggatccttaattattagcagctttaacctg

SEQ ID NO: 86 - DNA, 38 bp, Artificial Sequence; identical to SEQ ID NO: 66
  Description of Artificial Sequence Synthetic Gevo-288 primer

SEQ ID NO: 87 - DNA, 36 bp, Artificial Sequence
  Description of Artificial Sequence Synthetic Gevo-300 primer
gactttggatccttaaatatagtgttcttctttcag

SEQ ID NO: 88 - DNA, 37 bp, Artificial Sequence; identical to SEQ ID NO: 68
  Description of Artificial Sequence Synthetic Gevo-292 primer

SEQ ID NO: 89 - DNA, 41 bp, Artificial Sequence
  Description of Artificial Sequence Synthetic Gevo-301 primer
attttcggatccttaaaatgattttatatagatatctttta

SEQ ID NO: 90 - DNA, 37 bp, Artificial Sequence
  Description of Artificial Sequence Synthetic Gevo-302 primer
cttatagtcgacatggattttaacttaacagatattc

SEQ ID NO: 91 - DNA, 36 bp, Artificial Sequence
  Description of Artificial Sequence Synthetic Gevo-303 primer
ccgccaggatccttaacgtaacagagcaccgccggt

SEQ ID NO: 92 - DNA, 36 bp, Artificial Sequence
  Description of Artificial Sequence Synthetic Gevo-304 primer
cggaaagtcgacatggatttagcagaatacaaaggc

SEQ ID NO: 93 - DNA, 34 bp, Artificial Sequence
  Description of Artificial Sequence Synthetic Gevo-305 primer
ctttgtggatccttatgcaatgcctttctgtttc

SEQ ID NO: 94 - DNA, 37 bp, Artificial Sequence
  Description of Artificial Sequence Synthetic Gevo-306 primer
caaactgaattcatggaaatattggtatgtgtcaaac

SEQ ID NO: 95 - DNA, 35 bp, Artificial Sequence
  Description of Artificial Sequence Synthetic Gevo-307 primer
accaacggatccttaaatgattttctgggcaacca

SEQ ID NO: 96 - DNA, 34 bp, Artificial Sequence
  Description of Artificial Sequence Synthetic Gevo-273 primer
gttacagtcgacatgtctcagaacgtttacattg

SEQ ID NO: 97 - DNA, 35 bp, Artificial Sequence
  Description of Artificial Sequence Synthetic Gevo-274 primer
gataacggatcctcatatcttttcaatgacaatag

SEQ ID NO: 98 - DNA, 34 bp, Artificial Sequence
  Description of Artificial Sequence Synthetic PflA_forw primer
cattgaattcatgtcagttattggtcgcattcac

SEQ ID NO: 99 - DNA, 37 bp, Artificial Sequence
  Description of Artificial Sequence Synthetic PflA_Rev primer
cattgtcgacttagaacattaccttatgaccgtactg

SEQ ID NO: 100 - DNA, 37 bp, Artificial Sequence
  Description of Artificial Sequence Synthetic PflB_forw primer
cattgaattcatgtccgagcttaatgaaaagttagcc

SEQ ID NO: 101 - DNA, 36 bp, Artificial Sequence
  Description of Artificial Sequence Synthetic PflB_Rev primer
cattgtcgacttacatagattgagtgaaggtacgag

SEQ ID NO: 102 - DNA, 39 bp, Artificial Sequence
  Description of Artificial Sequence Synthetic fdh1_forw primer
cattgaattcatgaagatcgttttagtcttatatggtgc

SEQ ID NO: 103 - DNA, 37 bp, Artificial Sequence
  Description of Artificial Sequence Synthetic fdh1_rev primer
cattgtcgacttatttcttatcgtgtttaccgtaagc

SEQ ID NO: 104 - DNA, 38 bp, Artificial Sequence
  Description of Artificial Sequence Synthetic KlALD6_right3 primer
gttaggatccttaatccaacttgatcctgacggccttg

SEQ ID NO: 105 - DNA, 43 bp, Artificial Sequence
  Description of Artificial Sequence Synthetic KlALD6_Left5 primer
ccaagtcgacatgtcctctacaattgctgagaaattgaacctc

SEQ ID NO: 106 - DNA, 40 bp, Artificial Sequence
  Description of Artificial Sequence Synthetic KlACS1_Right3 primer
gttagcggccgcttataatttcacggaatcgatcaagtgc

SEQ ID NO: 107 - DNA, 37 bp, Artificial Sequence
  Description of Artificial Sequence Synthetic KlACS1_Left5 primer
ccaagctagcatgtctcctgctgttgataccgcttcc

SEQ ID NO: 108 - DNA, 49 bp, Artificial Sequence
  Description of Artificial Sequence Synthetic KlACS2_right3 primer
ggttggatccttatttcttctgctgactgaaaaattgattttctactgc

SEQ ID NO: 109 - DNA, 35 bp, Artificial Sequence
  Description of Artificial Sequence Synthetic KlACS2_Left5 primer
ccaagaattcatgtcgtcggataaattgcataagg

SEQ ID NO: 110 - DNA, 26 bp, Artificial Sequence
  Description of Artificial Sequence Synthetic Gevo-350 oligonucleotide
cttaaattctacttttatagttagtc

SEQ ID NO: 111 - DNA, 20 bp, Artificial Sequence
  Description of Artificial Sequence Synthetic Gevo-352 oligonucleotide
ccttccttttcggttagagc

SEQ ID NO: 112 - DNA, 31 bp, Artificial Sequence
  Description of Artificial Sequence Synthetic Gevo-479 primer
catgccgtcgacatgtcgccctctgccgtac

SEQ ID NO: 113 - DNA, 38 bp, Artificial Sequence
  Description of Artificial Sequence Synthetic Gevo-480 primer
gattaagcggccgcttacaacttgaccgaatcaattag

SEQ ID NO: 114 - DNA, 37 bp, Artificial Sequence
  Description of Artificial Sequence Synthetic Gevo-483 primer
gatgaagtcgacatgacaatcaaggaacataaagtag

SEQ ID NO: 115 - DNA, 39 bp, Artificial Sequence
  Description of Artificial Sequence Synthetic Gevo-484 primer
gttaaaggatccttatttctttttttgagagaaaaattg

SEQ ID NO: 116 - DNA, 44 bp, Artificial Sequence
  Description of Artificial Sequence Synthetic Gevo-606 primer
ttttgtcgacactagtatgtcagaacgtttcccaaatgacgtgg

SEQ ID NO: 117 - DNA, 39 bp, Artificial Sequence
  Description of Artificial Sequence Synthetic Gevo-607 primer
ttttctcgagttacgccagacgcgggttaactttatctg

SEQ ID NO: 118 - DNA, 40 bp, Artificial Sequence
  Description of Artificial Sequence Synthetic Gevo-609 primer
ttttctcgagttacatcaccagacggcgaatgtcagacag

SEQ ID NO: 119 - DNA, 46 bp, Artificial Sequence
  Description of Artificial Sequence Synthetic Gevo-610 primer
ttttgtcgacactagtatgagtactgaaatcaaaactcaggtcgtg

SEQ ID NO: 120 - DNA, 37 bp, Artificial Sequence
  Description of Artificial Sequence Synthetic Gevo-611 primer
ttttctcgagttacttcttcttcgctttcgggttcgg

SEQ ID NO: 121 - DNA, 21 bp, Artificial Sequence
  Description of Artificial Sequence Synthetic Gevo-616 oligonucleotide
ctatttaccaggctaaattcc

SEQ ID NO: 122 - DNA, 22 bp, Artificial Sequence
  Description of Artificial Sequence Synthetic Gevo-617 oligonucleotide
tgaaggtaaaaacatcgcgcac

SEQ ID NO: 123 - DNA, 23 bp, Artificial Sequence
  Description of Artificial Sequence Synthetic Gevo-618 oligonucleotide
cgtggcttcctgatcggcggtac

SEQ ID NO: 124 - DNA, 24 bp, Artificial Sequence
  Description of Artificial Sequence Synthetic Gevo-619 oligonucleotide
caccagcggctggcgtgaaagaag

SEQ ID NO: 125 - DNA, 24 bp, Artificial Sequence
  Description of Artificial Sequence Synthetic Gevo-620 oligonucleotide
gaagtggaactgggccgcatccag

SEQ ID NO: 126 - DNA, 22 bp, Artificial Sequence
  Description of Artificial Sequence Synthetic Gevo-621 oligonucleotide
gacgtggttgaaatgttcgacc

SEQ ID NO: 127 - DNA, 35 bp, Artificial Sequence
  Description of Artificial Sequence Synthetic Gevo-637 primer
ttttgagctcgccgatcccattaccgacatttggg

SEQ ID NO: 128 - DNA, 96 bp, Artificial Sequence
  Description of Artificial Sequence Synthetic Gevo-638 primer
aaagtcgacaccgatatacctgtatgtgtcaccaccaatgtatctataagtatccatgctagccctaggtttatgtgatgattgattgattgattg

SEQ ID NO: 129 - DNA, 36 bp, Artificial Sequence
  Description of Artificial Sequence Synthetic Gevo-639 primer
ttttctcgagactagtatgtctgaaattactttggg

SEQ ID NO: 130 - DNA, 36 bp, Artificial Sequence
  Description of Artificial Sequence Synthetic Gevo-640 primer
ttttggatccttattgcttagcgttggtagcagcag

SEQ ID NO: 131 - DNA, 27 bp, Artificial Sequence
  Description of Artificial Sequence Synthetic Gevo-641 oligonucleotide
gttatcttggctgatgcttgttgttcc

SEQ ID NO: 132 - DNA, 41 bp, Artificial Sequence
  Description of Artificial Sequence Synthetic Gevo-642 oligonucleotide
ttttgtcgacactagtatgagtactgaaatcaaaactcagg

SEQ ID NO: 133 - DNA, 33 bp, Artificial Sequence
  Description of Artificial Sequence Synthetic Gevo-643 primer
ccaagtcgacatgactaagctacactttgacac

SEQ ID NO: 134 - DNA, 27 bp, Artificial Sequence
  Description of Artificial Sequence Synthetic Gevo-644 primer
gtcggtaagagtgttgctgtggactcg

SEQ ID NO: 135 - DNA, 42 bp, Artificial Sequence
  Description of Artificial Sequence Synthetic Gevo-646 oligonucleotide
ccaaggatccttacaacttaattctgacagcttttacttcag

SEQ ID NO: 136 - DNA, 49 bp, Artificial Sequence
  Description of Artificial Sequence Synthetic Gevo-653 primer
ttttgtcgacactagtatggctatcgaaatcaaagtaccggacatcggg

SEQ ID NO: 137 - DNA, 25 bp, Artificial Sequence
  Description of Artificial Sequence Synthetic Gevo-654 oligonucleotide
cttccatagaagctttgtcgccttc

SEQ ID NO: 138 - DNA, 25 bp, Artificial Sequence
  Description of Artificial Sequence Synthetic Gevo-655 oligonucleotide
gtgcaatatcatatagaagtcatcg

SEQ ID NO: 139 - DNA, 48 bp, Artificial Sequence
  Description of Artificial Sequence Synthetic Gevo-656 primer
ttttctcgaggctagcatggcatcgtacccagagcacaccattattgg

SEQ ID NO: 140 - DNA, 40 bp, Artificial Sequence
  Description of Artificial Sequence Synthetic Gevo-657 primer
ttttggatcctcacaatagcatttccaaaggattttcaat

SEQ ID NO: 141 - DNA, 45 bp, Artificial Sequence
  Description of Artificial Sequence Synthetic Gevo-658 primer
ttttctcgagactagtatggtcatcatcggtggtggccctgctgg

SEQ ID NO: 142 - DNA, 36 bp, Artificial Sequence
  Description of Artificial Sequence Synthetic Gevo-659 primer
ttttggatcctcaacaatgaatagctttatcatagg

SEQ ID NO: 143 - DNA, 47 bp, Artificial Sequence
  Description of Artificial Sequence Synthetic Gevo-660 primer
ttttctcgagactagtatggcaactttaaaaacaactgataagaagg

SEQ ID NO: 144 - DNA, 35 bp, Artificial Sequence
  Description of Artificial Sequence Synthetic Gevo-661 primer
ttttagatctttaatccctagaggcaaaaccttgc

SEQ ID NO: 145 - DNA, 44 bp, Artificial Sequence
  Description of Artificial Sequence Synthetic Gevo-662 primer
ttttctcgagactagtatggcggaagaattggaccgtgatgatg

SEQ ID NO: 146 - DNA, 38 bp, Artificial Sequence
  Description of Artificial Sequence Synthetic Gevo-663 primer
tttggatccttattcaattgacaagacttctttgacag

SEQ ID NO: 147 - DNA, 48 bp, Artificial Sequence
  Description of Artificial Sequence Synthetic Gevo-664 primer
ttttctcgagactagtatgttacttgctgtaaagacattttcaatgcc

SEQ ID NO: 148 - DNA, 40 bp, Artificial Sequence
  Description of Artificial Sequence Synthetic Gevo-665 primer
ttttggatcctcaaaatgattctaactcccttacgtaatc

SEQ ID NO: 149 - DNA, 27 bp, Artificial Sequence
  Description of Artificial Sequence Synthetic Gevo-666 oligonucleotide
ggtagaattaccaaggctgacattgag

SEQ ID NO: 150 - DNA, 21 bp, Artificial Sequence
  Description of Artificial Sequence Synthetic Gevo-667 oligonucleotide
cattggtggaggaatcatcgg

SEQ ID NO: 151 - DNA, 24 bp, Artificial Sequence
  Description of Artificial Sequence Synthetic Gevo-668 oligonucleotide
caccaatacaagaacgaggacgcc

SEQ ID NO: 152 - DNA, 28 bp, Artificial Sequence
  Description of Artificial Sequence Synthetic Gevo-669 oligonucleotide
tggtacggttccattccagggttaaagg

SEQ ID NO: 153 - DNA, 25 bp, Artificial Sequence
  Description of Artificial Sequence Synthetic Gevo-670 oligonucleotide
gggtgatgtgctagcatacctaggg

SEQ ID NO: 154 - DNA, 1197 bp, Artificial Sequence
  Description of Artificial Sequence Synthetic ERG10 polynucleotide
atgtctcagaacgtttacattgtatcgactgccagaaccccaattggttcattccagggttctctatcctccaagacagcagtggaattgggtgctgttgctttaaaaggcgccttggctaaggttccagaattggatgcatccaaggattttgacgaaattatttttggtaacgttctttctgccaatttgggccaagctccggccagacaagttgctttggctgccggtttgagtaatcatatcgttgcaagcacagttaacaaggtctgtgcatccgctatgaaggcaatcattttgggtgctcaatccatcaaatgtggtaatgctgatgttgtcgtagctggtggttgtgaatctatgactaacgcaccatactacatgccagcagcccgtgcgggtgccaaatttggccaaactgttcttgttgatggtgtcgaaagagatgggttgaacgatgcgtacgatggtctagccatgggtgtacacgcagaaaagtgtgcccgtgattgggatattactagagaacaacaagacaattttgccatcgaatcctaccaaaaatctcaaaaatctcaaaaggaaggtaaattcgacaatgaaattgtacctgttaccattaagggatttagaggtaagcctgatactcaagtcacgaaggacgaggaacctgctagattacacgttgaaaaattgagatctgcaaggactgttttccaaaaagaaaacggtactgttactgccgctaacgcttctccaatcaacgatggtgctgcagccgtcatcttggtttccgaaaaagttttgaaggaaaagaatttgaagcctttggctattatcaaaggttggggtgaggccgctcatcaaccagctgattttacatgggctccatctcttgcagttccaaaggctttgaaacatgctggcatcgaagacatcaattctgttgattactttgaattcaatgaagccttttcggttgtcggtttggtgaacactaagattttgaagctagacccatctaaggttaatgtatatggtggtgctgttgctctaggtcacccattgggttgttctggtgctagagtggttgttacactgctatccatcttacagcaagaaggaggtaagatcggtgttgccgccatttgtaatggtggtggtggtgcttcctctattgtcattgaaaagatatga

SEQ ID NO: 155 - DNA, 849 bp, Artificial Sequence
  Description of Artificial Sequence Synthetic Cb-hbd polynucleotide
atgaaaaagatttttgtacttggagcaggaacaatgggtgctggtatcgttcaagcattcgctcaaaaaggttgtgaagtaattgtaagagacataaaggaagaatttgttgacagaggaatagctggaatcactaaaggattagaaaagcaagttgctaaaggaaaaatgtctgaagaagataaagaagctatactttcaagaatttcaggaacaactgatatgaaattagctgctgactgtgatttagtagttgaagctgcaatcgaaaacatgaaaattaagaaggaaatcttcgctgaattagatggaatttgtaagccagaagcgattttagcttcaaacacttcatctttatcaattactgaagttgcttcagctacaaagagacctgataaagttatcggaatgcatttctttaatccagctccagtaatgaagcttgttgaaattattaaaggaatagctacttctcaagaaacttttgatgctgttaaggaattatcagttgctattggaaaagaaccagtagaagttgcagaagctccaggattcgttgtaaacagaatattaatcccaatgattaacgaagcttcatttatcctacaagaaggaatagcttcagttgaagatattgatacagctatgaaatatggtgctaaccatccaatgggacctttagctttaggagatcttattggattagacgtttgcttagctatcatggatgttttattcactgaaacaggtgataacaagtacagagctagcagcatattaagaaaatatgttagagctggatggcttggaagaaaatcaggaaaaggattctatgattattctaaataa

SEQ ID NO: 156 - DNA, 786 bp, Artificial Sequence
  Description of Artificial Sequence Synthetic Cb-crt polynucleotide
atggaattaaaaaatgttattcttgaaaaagaagggcatttagctattgttacaatcaatagaccaaaggcattaaatgcattgaattcagaaacactaaaagatttaaatgttgttttagatgatttagaagcagacaacaatgtgtatgcagttatagttacaggtgctggtgagaaatcttttgttgctggagcagatatttcagaaatgaaagatcttaatgaagaacaaggtaaagaatttggtattttaggaaacaatgtcttcagaagattagaaaaattggataagccagttatcgcagctatatcaggatttgctcttggtggtggatgtgaacttgctatgtcatgtgacataagaatagcttcagttaaagctaaatttggtcaaccagaagcaggacttggaataactccaggatttggtggaactcaaagattagctagaattgtagggccaggaaaagctaaagaattaatttatacttgtgaccttataaatgcagaagaagcttatagaataggtttagttaataaagtagttgaattagaaaaattgatggaagaagcaaaagcaatggctaacaagattgcagctaatgctccaaaagcagttgcatattgtaaagatgctatagacagaggaatgcaagttgatatagatgcagctatattaatagaagcagaagactttggaaagtgctttgcaacagaagatcaaacagaaggaatgactgcgttcttagaaagaagagcagaaaagaattttcaaaataaataa

SEQ ID NO: 157 - DNA, 1140 bp, Artificial Sequence
  Description of Artificial Sequence Synthetic Cb-bcd polynucleotide
atgaatttccaattaactagagaacaacaattagtacaacaaatggttagagaattcgcagtaaatgaagttaagccaatagctgctgaaatcgacgaatcagaaagattccctatggaaaacgttgaaaaaatggctaagcttaaaatgatgggtatcccattttctaaagaatttggtggagcaggcggagatgttctttcatatataatatctgtggaagaattatcaaaagtttgtggtactacaggagttattctttcagcgcatacatcattatgtgcatcagtaattaatgaaaatggaactaacgaacaaagagcaaaatatttgccagatctttgtagtggtaagaaaatcggtgctttcggattaacagaaccaggcgctggtacagatgctgcaggacaacaaacaactgctgtattagaaggagaccattatgtattaaatggttcaaaaatcttcataacaaatggtggagttgctgaaactttcataatatttgctatgacagataagagtcaaggaacaaaaggaatttctgcattcatagtagaaaagtcattcccaggattctcaataggaaaattagaaaacaagatggggatcagagcatcttcaactactgagttagttatggaaaactgtatagtaccaaaagaaaacctacttagcaaagaaggtaagggatttggtatagcaatgaaaactcttgatggaggaagaattggtatagctgctcaagctttaggtattgcagaaggagcttttgaagaagctgttaactatatgaaagaaagaaaacaatttggtaaaccattatcagcattccaaggattacaatggtatatagctgaaatggatgttaaaatccaagctgctaaatacttagtatacctagctgcaacaaagaagcaagctggtgagccttactcagtggatgctgcaagagctaaattatttgcggcagatgttgcaatggaagttacaactaaagcagttcaaatctttggtggatatggttacactaaggaatacccagtagaaagaatgatgagagatgctaaaatatgcgaaatctacgaaggaacttcagaagttcaaaagatggttatcgcaggaagcattttaagatag

SEQ ID NO: 158 - DNA, 1140 bp, Artificial Sequence; identical to SEQ ID NO: 157
  Description of Artificial Sequence Synthetic Cb-etfA polynucleotide

SEQ ID NO: 159 - DNA, 780 bp, Artificial Sequence
  Description of Artificial Sequence Synthetic Cb-etfB polynucleotide
atgaatatagtagtttgtgtaaaacaagttccagatactacagcagtaaaaattgatcctaaaactggtacattaataagagatggtgttccatcaataatgaatccagaggataaacacgctttagaaggtgcattacaattaaaagaaaaagttggaggaaaagttactgtaataagtatgggacttccaatggctaaagcagtattaagagaagcattatgtatgggagctgatgaagctgtcctattaacagatagagcacttggaggagcagatactttagcaacttcaaaggcacttgcaggagtaatagctaagttagattatgatttggtatttgctggaagacaagcaattgatggagatactgcacaagtaggaccagaaatagcagaacatttaaacattccgcaagtaacttacgttcaagacgttaaagttgaaggaaatacattaatagtaaatagagcactagaagatggacatcaagtagtagaagttaaaactccatgtctattaactgcaatcgaagaattaaatgaaactagatatatgaatgttgtagatatattcgaaacttcagatgatgaaatcaaagttatgagcgcagctgatatagatgtagatgtagctgaattagggcttaaaggctcacctacaaaggttaagaagtcaatgactaaggaagttaaaggtgcaggagaaatcgtaagagaagcacctaaaaatgcagcatactatgttgtaggaaaattaaaagaaaaacactacatctaa

SEQ ID NO: 160 - DNA, 1167 bp, Artificial Sequence
  Description of Artificial Sequence Synthetic Cb-adhA polynucleotide
atggcacgttttactttaccaagagacatttatcatggagaaggagcacttgaggcacttaaaactttaaaaggtaagaaagctttcttagtagttggtggcggatcaatgaaaagatttggatttcttaaacaagttgaagattatttaaaagaagcaggaatggaagtagaattatttgaaggtgttgaaccagatccatcagtggaaacagtaatgaaaggcgcagaagctatgagaaactttgagcctgattggatagttgcaatgggtggaggatcaccaattgatgctgcaaaggctatgtggatattctacgaatacccagattttacttttgaacaagcagttgttccatttggattaccagaccttagacaaaaagctaagtttgtagctattccatcaacaagcggtacagctacagaagttacagcattctcagttatcacaaattattcagaaaaaattaaatatcctttagctgattttaacataactccagatatagcaatagttgatccagcacttgctcaaactatgccaaaaactttaacagctcatactggaatggatgcattaactcacgctatagaagcatacactgcatcacttcaatcaaatttctcagatccattagcaattaaagctgtagaaatggttcaagaaaatttaatcaaatcatttgaaggagataaagaagctagaaatctaatgcatgaagctcaatgtttagctggaatggcattttctaatgcattacttggaatagttcactcaatggctcataaggttggtgctgtattccatattcctcatggatgtgcaaatgctatatttttaccatatgtaattgagtataacagaacaaaatgcgaaaatagatatggagatattgcgagagccttaaaattaaaaggaaacaatgatgccgagttaactgattcattaattgaattaattaatggattaaatgataagttagagattcctcactcaatgaaagagtatggagttactgaagaagattttaaagctaatctttcatttatcgctcataacgcagtattagatgcatgcacaggatcaaatcctagagaaatagatgatgctacaatggaaaaattatttgaatgcacatactatggaactaaagttaatttgtaa

SEQ ID NO: 161 - DNA, 1407 bp, Artificial Sequence
  Description of Artificial Sequence Synthetic Cb-aldh polynucleotide
atgaataaagacacactaatacctacaactaaagatttaaaagtaaaaacaaatggtgaaaacattaatttaaagaactacaaggataattcttcatgtttcggagtattcgaaaatgttgaaaatgctataagcagcgctgtacacgcacaaaagatattatcccttcattatacaaaagagcaaagagaaaaaatcataactgagataagaaaggccgcattacaaaataaagaggtcttggctacaatgattctagaagaaacacatatgggaagatatgaggataaaatattaaaacatgaattggtagctaaatatactcctggtacagaagatttaactactactgcttggtcaggtgataatggtcttacagttgtagaaatgtctccatatggtgttataggtgcaataactccttctacgaatccaactgaaactgtaatatgtaatagcataggcatgatagctgctggaaatgctgtagtatttaacggacacccatgcgctaaaaaatgtgttgcctttgctgttgaaatgataaataaggcaattatttcatgtggcggtcctgaaaatctagtaacaactataaaaaatccaactatggagtctctagatgcaattattaagcatccttcaataaaacttctttgcggaactgggggtccaggaatggtaaaaaccctcttaaattctggtaagaaagctataggtgctggtgctggaaatccaccagttattgtagatgatactgctgatatagaaaaggctggtaggagcatcattgaaggctgttcttttgataataatttaccttgtattgcagaaaaagaagtatttgtttttgagaatgttgcagatgatttaatatctaacatgctaaaaaataatgctgtaattataaatgaagatcaagtatcaaaattaatagatttagtattacaaaaaaataatgaaactcaagaatactttataaacaaaaaatgggtaggaaaagatgcaaaattattcttagatgaaatagatgttgagtctccttcaaatgttaaatgcataatctgcgaagtaaatgcaaatcatccatttgttatgacagaactcatgatgccaatattgccaattgtaagagttaaagatatagatgaagctattaaatatgcaaagatagcagaacaaaatagaaaacatagtgcctatatttattctaaaaatatagacaacctaaatagatttgaaagagaaatagatactactatttttgtaaagaatgctaaatcttttgctggtgttggttatgaagcagaaggatttacaactttcactattgctggatctactggtgagggaataacctctgcaaggaattttacaagacaaagaagatgtgtacttgccggctaa

SEQ ID NO: 162 - DNA, 1179 bp, Artificial Sequence
  Description of Artificial Sequence Synthetic Ca-thl polynucleotide
atgaaagaagttgtaatagctagtgcagtaagaacagcgattggatcttatggaaagtctcttaaggatgtaccagcagtagatttaggagctacagctataaaggaagcagttaaaaaagcaggaataaaaccagaggatgttaatgaagtcattttaggaaatgttcttcaagcaggtttaggacagaatccagcaagacaggcatcttttaaagcaggattaccagttgaaattccagctatgactattaataaggtttgtggttcaggacttagaacagttagcttagcagcacaaattataaaagcaggagatgctgacgtaataatagcaggtggtatggaaaatatgtctagagctccttacttagcgaataacgctagatggggatatagaatgggaaacgctaaatttgttgatgaaatgatcactgacggattgtgggatgcatttaatgattaccacatgggaataacagcagaaaacatagctgagagatggaacatttcaagagaagaacaagatgagtttgctcttgcatcacaaaaaaaagctgaagaagctataaaatcaggtcaatttaaagatgaaatagttcctgtagtaattaaaggcagaaagggagaaactgtagttgatacagatgagcaccctagatttggatcaactatagaaggacttgcaaaattaaaacctgccttcaaaaaagatggaacagttacagctggtaatgcatcaggattaaatgactgtgcagcagtacttgtaatcatgagtgcagaaaaagctaaagagcttggagtaaaaccacttgctaagatagtttcttatggttcagcaggagttgacccagcaataatgggatatggacctttctatgcaacaaaagcagctattgaaaaagcaggttggacagttgatgaattagatttaatagaatcaaatgaagcttttgcagctcaaagtttagcagtagcaaaagatttaaaatttgatatgaataaagtaaatgtaaatggaggagctattgcccttggtcatccaattggagcatcaggtgcaagaatactcgttactcttgtacacgcaatgcaaaaaagagatgcaaaaaaaggcttagcaactttatgtataggtggcggacaaggaacagcaatattgctagaaaagtgctag

SEQ ID NO: 163 - DNA, 849 bp, Artificial Sequence
  Description of Artificial Sequence Synthetic Ca-hbd polynucleotide
atgaaaaaggtatgtgttataggtgcaggtactatgggttcaggaattgctcaggcatttgcagctaaaggatttgaagtagtattaagagatattaaagatgaatttgttgatagaggattagattttatcaataaaaatctttctaaattagttaaaaaaggaaagatagaagaagctactaaagttgaaatcttaactagaatttccggaacagttgaccttaatatggcagctgattgcgatttagttatagaagcagctgttgaaagaatggatattaaaaagcagatttttgctgacttagacaatatatgcaagccagaaacaattcttgcatcaaatacatcatcactttcaataacagaagtggcatcagcaactaaaagacctgataaggttataggtatgcatttctttaatccagctcctgttatgaagcttgtagaggtaataagaggaatagctacatcacaagaaacttttgatgcagttaaagagacatctatagcaataggaaaagatcctgtagaagtagcagaagcaccaggatttgttgtaaatagaatattaataccaatgattaatgaagcagttggtatattagcagaaggaatagcttcagtagaagacatagataaagctatgaaacttggagctaatcacccaatgggaccattagaattaggtgattttataggtcttgatatatgtcttgctataatggatgttttatactcagaaactggagattctaagtatagaccacatacattacttaagaagtatgtaagagcaggatggcttggaagaaaatcaggaaaaggtttctacgattattcaaaataa

SEQ ID NO: 164 - DNA, 786 bp, Artificial Sequence
  Description of Artificial Sequence Synthetic Ca-crt polynucleotide
atggaactaaacaatgtcatccttgaaaaggaaggtaaagttgctgtagttaccattaacagacctaaagcattaaatgcgttaaatagtgatacactaaaagaaatggattatgttataggtgaaattgaaaatgatagcgaagtacttgcagtaattttaactggagcaggagaaaaatcatttgtagcaggagcagatatttctgagatgaaggaaatgaataccattgaaggtagaaaattcgggatacttggaaataaagtgtttagaagattagaacttcttgaaaagcctgtaatagcagctgttaatggttttgctttaggaggcggatgcgaaatagctatgtcttgtgatataagaatagcttcaagcaacgcaagatttggtcaaccagaagtaggtctcggaataacacctggttttggtggtacacaaagactttcaagattagttggaatgggcatggcaaagcagcttatatttactgcacaaaatataaaggcagatgaagcattaagaatcggacttgtaaataaggtagtagaacctagtgaattaatgaatacagcaaaagaaattgcaaacaaaattgtgagcaatgctccagtagctgttaagttaagcaaacaggctattaatagaggaatgcagtgtgatattgatactgctttagcatttgaatcagaagcatttggagaatgcttttcaacagaggatcaaaaggatgcaatgacagctttcatagagaaaagaaaaattgaaggcttcaaaaatagatag

SEQ ID NO: 165 - DNA, 1140 bp, Artificial Sequence
  Description of Artificial Sequence Synthetic Ca-bcd polynucleotide
atggattttaatttaacaagagaacaagaattagtaagacagatggttagagaatttgctgaaaatgaagttaaacctatagcagcagaaattgatgaaacagaaagatttccaatggaaaatgtaaagaaaatgggtcagtatggtatgatgggaattccattttcaaaagagtatggtggcgcaggtggagatgtattatcttatataatcgccgttgaggaattatcaaaggtttgcggtactacaggagttattctttcagcacatacatcactttgtgcttcattaataaatgaacatggtacagaagaacaaaaacaaaaatatttagtacctttagctaaaggtgaaaaaataggtgcttatggattgactgagccaaatgcaggaacagattctggagcacaacaaacagtagctgtacttgaaggagatcattatgtaattaatggttcaaaaatattcataactaatggaggagttgcagatacttttgttatatttgcaatgactgacagaactaaaggaacaaaaggtatatcagcatttataatagaaaaaggcttcaaaggtttctctattggtaaagttgaacaaaagcttggaataagagcttcatcaacaactgaacttgtatttgaagatatgatagtaccagtagaaaacatgattggtaaagaaggaaaaggcttccctatagcaatgaaaactcttgatggaggaagaattggtatagcagctcaagctttaggtatagctgaaggtgctttcaacgaagcaagagcttacatgaaggagagaaaacaatttggaagaagccttgacaaattccaaggtcttgcatggatgatggcagatatggatgtagctatagaatcagctagatatttagtatataaagcagcatatcttaaacaagcaggacttccatacacagttgatgctgcaagagctaagcttcatgctgcaaatgtagcaatggatgtaacaactaaggcagtacaattatttggtggatacggatatacaaaagattatccagttgaaagaatgatgagagatgctaagataactgaaatatatgaaggaacttcagaagttcagaaattagttatttcaggaaaaatttttagataa

SEQ ID NO: 166 - DNA, 1011 bp, Artificial Sequence
  Description of Artificial Sequence Synthetic Ca-etfA polynucleotide
atgaataaagcagattacaagggcgtatgggtgtttgctgaacaaagagacggagaattacaaaaggtatcattggaattattaggtaaaggtaaggaaatggctgagaaattaggcgttgaattaacagctgttttacttggacataatactgaaaaaatgtcaaaggatttattatctcatggagcagataaggttttagcagcagataatgaacttttagcacatttttcaacagatggatatgctaaagttatatgtgatttagttaatgaaagaaagccagaaatattattcataggagctactttcataggaagagatttaggaccaagaatagcagcaagactttctactggtttaactgctgattgtacatcacttgacatagatgtagaaaatagagatttattggctacaagaccagcgtttggtggaaatttgatagctacaatagtttgttcagaccacagaccacaaatggctacagtaagacctggtgtgtttgaaaaattacctgttaatgatgcaaatgtttctgatgataaaatagaaaaagttgcaattaaattaacagcatcagacataagaacaaaagtttcaaaagttgttaagcttgctaaagatattgcagatatcggagaagctaaggtattagttgctggtggtagaggagttggaagcaaagaaaactttgaaaaacttgaagagttagcaagtttacttggtggaacaatagccgcttcaagagcagcaatagaaaaagaatgggttgataaggaccttcaagtaggtcaaactggtaaaactgtaagaccaactctttatattgcatgtggtatatcaggagctatccagcatttagcaggtatgcaagattcagattacataattgctataaataaagatgtagaagccccaataatgaaggtagcagatttggctatagttggtgatgtaaataaagttgtaccagaattaatagctcaagttaaagctgctaataattaa

SEQ ID NO: 167 - DNA, 780 bp, Artificial Sequence
  Description of Artificial Sequence Synthetic Ca-etfB polynucleotide
atgaatatagttgtttgtttaaaacaagttccagatacagcggaagttagaatagatccagttaagggaacacttataagagaaggagttccatcaataataaatccagatgataaaaacgcacttgaggaagctttagtattaaaagataattatggtgcacatgtaacagttataagtatgggacctccacaagctaaaaatgctttagtagaagctttggctatgggtgctgatgaagctgtacttttaacagatagagcatttggaggagcagatacacttgcgacttcacatacaattgcagcaggaattaagaagctaaaatatgatatagtttttgctggaaggcaggctatagatggagatacagctcaggttggaccagaaatagctgagcatcttggaatacctcaagtaacttatgttgagaaagttgaagttgatggagatactttaaagattagaaaagcttgggaagatggatatgaagttgttgaagttaagacaccagttcttttaacagcaattaaagaattaaatgttccaagatatatgagtgtagaaaaaatattcggagcatttgataaagaagtaaaaatgtggactgccgatgatatagatgtagataaggctaatttaggtcttaaaggttcaccaactaaagttaagaagtcatcaactaaagaagttaaaggacagggagaagttattgataagcctgttaaggaagcagctgcatatgttgtctcaaaattaaaagaagaacactatatttaa

SEQ ID NO: 168 - DNA, 2577 bp, Artificial Sequence
  Description of Artificial Sequence Synthetic Ca-adhE2 polynucleotide
atgaaagttacaaatcaaaaagaactaaaacaaaagctaaatgaattgagagaagcgcaaaagaagtttgcaacctatactcaagagcaagttgataaaatttttaaacaatgtgccatagccgcagctaaagaaagaataaacttagctaaattagcagtagaagaaacaggaataggtcttgtagaagataaaattataaaaaatcattttgcagcagaatatatatacaataaatataaaaatgaaaaaacttgtggcataatagaccatgacgattctttaggcataacaaaggttgctgaaccaattggaattgttgcagccatagttcctactactaatccaacttccacagcaattttcaaatcattaatttctttaaaaacaagaaacgcaatattcttttcaccacatccacgtgcaaaaaaatctacaattgctgcagcaaaattaattttagatgcagctgttaaagcaggagcacctaaaaatataataggctggatagatgagccatcaatagaactttctcaagatttgatgagtgaagctgatataatattagcaacaggaggtccttcaatggttaaagcggcctattcatctggaaaacctgcaattggtgttggagcaggaaatacaccagcaataatagatgagagtgcagatatagatatggcagtaagctccataattttatcaaagacttatgacaatggagtaatatgcgcttctgaacaatcaatattagttatgaattcaatatacgaaaaagttaaagaggaatttgtaaaacgaggatcatatatactcaatcaaaatgaaatagctaaaataaaagaaactatgtttaaaaatggagctattaatgctgacatagttggaaaatctgcttatataattgctaaaatggcaggaattgaagttcctcaaactacaaagatacttataggcgaagtacaatctgttgaaaaaagcgagctgttctcacatgaaaaactatcaccagtacttgcaatgtataaagttaaggattttgatgaagctctaaaaaaggcacaaaggctaatagaattaggtggaagtggacacacgtcatctttatatatagattcacaaaacaataaggataaagttaaagaatttggattagcaatgaaaacttcaaggacatttattaacatgccttcttcacagggagcaagcggagatttatacaattttgcgatagcaccatcatttactcttggatgcggcacttggggaggaaactctgtatcgcaaaatgtagagcctaaacatttattaaatattaaaagtgttgctgaaagaagggaaaatatgctttggtttaaagtgccacaaaaaatatattttaaatatggatgtcttagatttgcattaaaagaattaaaagatatgaataagaaaagagcctttatagtaacagataaagatctttttaaacttggatatgttaataaaataacaaaggtactagatgagatagatattaaatacagtatatttacagatattaaatctgatccaactattgattcagtaaaaaaaggtgctaaagaaatgcttaactttgaacctgatactataatctctattggtggtggatcgccaatggatgcagcaaaggttatgcacttgttatatgaatatccagaagcagaaattgaaaatctagctataaactttatggatataagaaagagaatatgcaatttccctaaattaggtacaaaggcgatttcagtagctattcctacaactgctggtaccggttcagaggcaacaccttttgcagttataactaatgatgaaacaggaatgaaataccctttaacttcttatgaattgaccccaaacatggcaataatagatactgaattaatgttaaatatgcctagaaaattaacagcagcaactggaatagatgcattagttcatgctatagaagcatatgtttcggttatggctacggattatactgatgaattagccttaagagcaataaaaatgatatttaaatatttgcctagagcctataaaaatgggactaacgacattgaagcaagagaaaaaatggcacatgcctctaatattgcggggatggcatttgcaaatgctttcttaggtgtatgccattcaatggctcataaacttggggcaatgcatcacgttccacatggaattgcttgtgctgtattaatagaagaagttattaaatataacgctacagactgtccaacaaagcaaacagcattccctcaatataaatctcctaatgctaagagaaaatatgctgaaattgcagagtatttgaatttaaagggtactagcgataccgaaaaggtaacagccttaatagaagctatttcaaagttaaagatagatttgagtattccacaaaatataagtgccgctggaataaataaaaaagatttttataatacgctagataaaatgtcagagcttgcttttgatgaccaatgtacaacagctaatcctaggtatccacttataagtgaacttaaggatatctatataaaatcattttaa

SEQ ID NO: 169 - DNA, 2589 bp, Artificial Sequence
  Description of Artificial Sequence Synthetic Ca-aad polynucleotide
atgaaagtcacaacagtaaaggaattagatgaaaaactcaaggtaattaaagaagctcaaaaaaaattctcttgttactcgcaagaaatggttgatgaaatctttagaaatgcagcaatggcagcaatcgacgcaaggatagagctagcaaaagcagctgttttggaaaccggtatgggcttagttgaagacaaggttataaaaaatcattttgcaggcgaatacatctataacaaatataaggatgaaaaaacctgcggtataattgaacgaaatgaaccctacggaattacaaaaatagcagaacctataggagttgtagctgctataatccctgtaacaaaccccacatcaacaacaatatttaaatccttaatatcccttaaaactagaaatggaattttcttttcgcctcacccaagggcaaaaaaatccacaatactagcagctaaaacaatacttgatgcagccgttaagagtggtgccccggaaaatataataggttggatagatgaaccttcaattgaactaactcaatatttaatgcaaaaagcagatataacccttgcaactggtggtccctcactagttaaatctgcttattcttccggaaaaccagcaataggtgttggtccgggtaacaccccagtaataattgatgaatctgctcatataaaaatggcagtaagttcaattatattatccaaaacctatgataatggtgttatatgtgcttctgaacaatctgtaatagtcttaaaatccatatataacaaggtaaaagatgagttccaagaaagaggagcttatataataaagaaaaacgaattggataaagtccgtgaagtgatttttaaagatggatccgtaaaccctaaaatagtcggacagtcagcttatactatagcagctatggctggcataaaagtacctaaaaccacaagaatattaataggagaagttacctccttaggtgaagaagaaccttttgcccacgaaaaactatctcctgttttggctatgtatgaggctgacaattttgatgatgctttaaaaaaagcagtaactctaataaacttaggaggcctcggccatacctcaggaatatatgcagatgaaataaaagcacgagataaaatagatagatttagtagtgccatgaaaaccgtaagaacctttgtaaatatcccaacctcacaaggtgcaagtggagatctatataattttagaataccaccttctttcacgcttggctgcggattttggggaggaaattctgtttccgagaatgttggtccaaaacatcttttgaatattaaaaccgtagctgaaaggagagaaaacatgctttggtttagagttccacataaagtatattttaagttcggttgtcttcaatttgctttaaaagatttaaaagatctaaagaaaaaaagagcctttatagttactgatagtgacccctataatttaaactatgttgattcaataataaaaatacttgagcacctagatattgattttaaagtatttaataaggttggaagagaagctgatcttaaaaccataaaaaaagcaactgaagaaatgtcctcctttatgccagacactataatagctttaggtggtacccctgaaatgagctctgcaaagctaatgtgggtactatatgaacatccagaagtaaaatttgaagatcttgcaataaaatttatggacataagaaagagaatatatactttcccaaaactcggtaaaaaggctatgttagttgcaattacaacttctgctggttccggttctgaggttactccttttgctttagtaactgacaataacactggaaataagtacatgttagcagattatgaaatgacaccaaatatggcaattgtagatgcagaacttatgatgaaaatgccaaagggattaaccgcttattcaggtatagatgcactagtaaatagtatagaagcatacacatccgtatatgcttcagaatacacaaacggactagcactagaggcaatacgattaatatttaaatatttgcctgaggcttacaaaaacggaagaaccaatgaaaaagcaagagagaaaatggctcacgcttcaactatggcaggtatggcatccgctaatgcatttctaggtctatgtcattccatggcaataaaattaagttcagaacacaatattcctagtggcattgccaatgcattactaatagaagaagtaataaaatttaacgcagttgataatcctgtaaaacaagccccttgcccacaatataagtatccaaacaccatatttagatatgctcgaattgcagattatataaagcttggaggaaatactgatgaggaaaaggtagatctcttaattaacaaaatacatgaactaaaaaaagctttaaatataccaacttcaataaaggatgcaggtgttttggaggaaaacttctattcctcccttgatagaatatctgaacttgcactagatgatcaatgcacaggcgctaatcctagatttcctcttacaagtgagataaaagaaatgtatataaattgttttaaaaaacaaccttaa

SEQ ID NO: 170 - DNA, 1167 bp, Artificial Sequence
  Description of Artificial Sequence Synthetic Ca-bdhA polynucleotide
atgctaagttttgattattcaataccaactaaagttttttttggaaaaggaaaaatagacgtaattggagaagaaattaagaaatatggctcaagagtgcttatagtttatggcggaggaagtataaaaaggaacggtatatatgatagagcaacagctatattaaaagaaaacaatatagctttctatgaactttcaggagtagagccaaatcctaggataacaacagtaaaaaaaggcatagaaatatgtagagaaaataatgtggatttagtattagcaatagggggaggaagtgcaatagactgttctaaggtaattgcagctggagtttattatgatggcgatacatgggacatggttaaagatccatctaaaataactaaagttcttccaattgcaagtatacttactctttcagcaacagggtctgaaatggatcaaattgcagtaatttcaaatatggagactaatgaaaagcttggagtaggacatgatgatatgagacctaaattttcagtgttagatcctacatatacttttacagtacctaaaaatcaaacagcagcgggaacagctgacattatgagtcacacctttgaatcttactttagtggtgttgaaggtgcttatgtgcaggacggtatagcagaagcaatcttaagaacatgtataaagtatggaaaaatagcaatggagaagactgatgattacgaggctagagctaatttgatgtgggcttcaagtttagctataaatggtctattatcacttggtaaggatagaaaatggagttgtcatcctatggaacacgagttaagtgcatattatgatataacacatggtgtaggacttgcaattttaacacctaattggatggaatatattctaaatgacgatacacttcataaatttgtttcttatggaataaatgtttggggaatagacaagaacaaagataactatgaaatagcacgagaggctattaaaaatacgagagaatactttaattcattgggtattccttcaaagcttagagaagttggaataggaaaagataaactagaactaatggcaaagcaagctgttagaaattctggaggaacaataggaagtttaagaccaataaatgcagaggatgttcttgagatatttaaaaaatcttat

SEQ ID NO: 171 - DNA, 1173 bp, Artificial Sequence
  Description of Artificial Sequence Synthetic Ca-bdhB polynucleotide
atggttgatttcgaatattcaataccaactagaatttttttcggtaaagataagataaatgtacttggaagagagcttaaaaaatatggttctaaagtgcttatagtttatggtggaggaagtataaagagaaatggaatatatgataaagctgtaagtatacttgaaaaaaacagtattaaattttatgaacttgcaggagtagagccaaatccaagagtaactacagttgaaaaaggagttaaaatatgtagagaaaatggagttgaagtagtactagctataggtggaggaagtgcaatagattgcgcaaaggttatagcagcagcatgtgaatatgatggaaatccatgggatattgtgttagatggctcaaaaataaaaagggtgcttcctatagctagtatattaaccattgctgcaacaggatcagaaatggatacgtgggcagtaataaataatatggatacaaacgaaaaactaattgcggcacatccagatatggctcctaagttttctatattagatccaacgtatacgtataccgtacctaccaatcaaacagcagcaggaacagctgatattatgagtcatatatttgaggtgtattttagtaatacaaaaacagcatatttgcaggatagaatggcagaagcgttattaagaacttgtattaaatatggaggaatagctcttgagaagccggatgattatgaggcaagagccaatctaatgtgggcttcaagtcttgcgataaatggacttttaacatatggtaaagacactaattggagtgtacacttaatggaacatgaattaagtgcttattacgacataacacacggcgtagggcttgcaattttaacacctaattggatggagtatattttaaataatgatacagtgtacaagtttgttgaatatggtgtaaatgtttggggaatagacaaagaaaaaaatcactatgacatagcacatcaagcaatacaaaaaacaagagattactttgtaaatgtactaggtttaccatctagactgagagatgttggaattgaagaagaaaaattggacataatggcaaaggaatcagtaaagcttacaggaggaaccataggaaacctaagaccagtaaacgcctccgaagtcctacaaatattcaaaaaatctgtgtaa

SEQ ID NO: 172 - DNA, 48 bp, Artificial Sequence
  Description of Artificial Sequence Synthetic AU1 tag oligonucleotide
atggatacttatagatacattggtggtgacacatacaggtatatcggt

SEQ ID NO: 173 - DNA, 33 bp, Artificial Sequence
  Description of Artificial Sequence Synthetic HA tag oligonucleotide
atgtacccatacgatgttcctgactatgcgggt

SEQ ID NO: 174 - DNA, 33 bp, Artificial Sequence
  Description of Artificial Sequence Synthetic myc tag oligonucleotide
atggaacaaaaactcatctcagaagaagatggt

SEQ ID NO: 175 - DNA, 403 bp, Artificial Sequence
  Description of Artificial Sequence Synthetic TEF1 promoter polynucleotide
catagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaaattttttttttgatttttttctctttcgatgacctcccattgatatttaagttaataaacggtcttcaatttctcaagtttcagtttcatttttcttgttctattacaactttttttacttcttgctcattagaaagaaagcatagcaatctaatctaagtttt

SEQ ID NO: 176 - DNA, 650 bp, Artificial Sequence
  Description of Artificial Sequence Synthetic TDH3 promoter polynucleotide
agtttatcattatcaatactcgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccagaacttagtttcga

SEQ ID NO: 177 - DNA, 493 bp, Artificial Sequence
  Description of Artificial Sequence Synthetic MET3 promoter polynucleotide
tttagtactaacagagacttttgtcacaactacatataagtgtacaaatatagtacagatatgacacacttgtagcgccaacgcgcatcctacggattgctgacagaaaaaaaggtcacgtgaccagaaaagtcacgtgtaattttgtaactcaccgcattctagcggtccctgtcgtgcacactgcactcaacaccataaaccttagcaacctccaaaggaaatcaccgtataacaaagccacagttttacaacttagtctcttatgaagttacttaccaatgagaaatagaggctctttctcgagaaatatgaatatggatatatatatatatatatatatatatatatatatatatgtaaacttggttcttttttagcttgtgatctctagcttgggtctctctctgtcgtaacagttgtgatatcgtttcttaacaattgaaaaggaactaagaaagtataataataacaagaataaagtataattaac

SEQ ID NO: 178 - DNA, 461 bp, Artificial Sequence
  Description of Artificial Sequence Synthetic CUP1 promoter polynucleotide
gccgatcccattaccgacatttgggcgctatacgtgcatatgttcatgtatgtatctgtatttaaaacacttttgtattatttttcctcatatatgtgtataggtttatacggatgatttaattattacttcaccaccctttatttcaggctgatatcttagccttgttactagttagaaaaagacatttttgctgtcagtcactgtcaagagattcttttgctggcatttcttctagaagcaaaaagagcgatgcgtcttttccgctgaaccgttccagcaaaaaagactaccaacgcaatatggattgtcagaatcatataaaagagaagcaaataactccttgtcttgtatcaattgcattataatatcttcttgttagtgcaatatcatatagaagtcatcgaaatagatattaagaaaaacaaactgtacaatcaatcaatcaatcatcacataaa

SEQ ID NO: 179 - DNA, 1197 bp, Artificial Sequence
  Description of Artificial Sequence Synthetic Ca-ter polynucleotide
atgatagtaaaagcaaagtttgtaaaaggatttatcagagatgtacatccttatggttgcagaagggaagtactaaatcaaatagattattgtaagaaggctattgggtttaggggaccaaagaaggttttaattgttggagcctcatctgggtttggtcttgctactagaatttcagttgcatttggaggtccagaagctcacacaattggagtatcctatgaaacaggagctacagatagaagaataggaacagcgggatggtataataacatattttttaaagaatttgctaaaaaaaaaggattagttgcaaaaaacttcattgaggatgccttttctaatgaaaccaaagataaagttattaagtatataaaggatgaatttggtaaaatagatttatttgtttatagtttagctgcgcctaggagaaaggactataaaactggaaatgtttatacttcaagaataaaaacaattttaggagattttgagggaccgactattgatgttgaaagagacgagattactttaaaaaaggttagtagtgctagcattgaagaaattgaagaaactagaaaggtaatgggtggagaggattggcaagagtggtgtgaagagctgctttatgaagattgtttttcggataaagcaactaccatagcatactcgtatataggatccccaagaacctacaagatatatagagaaggtactataggaatagctaaaaaggatcttgaagataaggctaagcttataaatgaaaaacttaacagagttataggtggtagagcctttgtgtctgtgaataaagcattagttacaaaagcaagtgcatatattccaacttttcctctttatgcagctattttatataaggtcatgaaagaaaaaaatattcatgaaaattgtattatgcaaattgagagaatgttttctgaaaaaatatattcaaatgaaaaaatacaatttgatgacaagggaagattaaggatggacgatttagagcttagaaaagacgttcaagacgaagttgatagaatatggagtaatattactcctgaaaattttaaggaattatctgattataagggatacaaaaaagaattcatgaacttaaacggttttgatctagatggggttgattatagtaaagacctggatatagaattattaagaaaattagaaccttaa

SEQ ID NO: 180 - DNA, 1194 bp, Artificial Sequence
  Description of Artificial Sequence Synthetic Ah-ter polynucleotide
atgatcattaaaccgaaagttcgtggcttcatttgtaccaccactcatccggttggctgtgaagctaatgtacgccgccagatcgcgtataccaaagcaaaaggcactatcgaaaacggccctaagaaagtgctggtgattggtgcgagcaccggttacggtctggcgtcccgcattgcagcggcgttcggtagcggcgccgcgaccctgggtgttttcttcgaaaaagcgggctccgaaactaaaaccgcgaccgcaggttggtacaactctgccgcgtttgacaaagccgccaaagaggctggcctgtatgcgaaatctattaacggtgacgcgttcagcaacgaatgccgtgctaaagtgatcgaactgatcaaacaggatctgggccaaattgatctggttgtttattctctggcctccccggttcgtaaactgccggataccggcgaagttgtgcgcagcgctctgaaacctattggtgaagtgtacaccacgaccgcaattgatactaataaggaccagattatcaccgcaaccgtcgagccggccaacgaggaagagatccagaataccatcactgtgatgggcggtcaagactgggaactgtggatggcagcactgcgcgacgcaggtgttctggcagacggtgcaaagagcgtcgcttactcttacatcggcactgacctgacttggccgatctactggcatggcaccctgggtcgcgcgaaagaggatctggatcgcgcagcggcagcgatccgcggtgatctggccggtaagggcggtactgcgcacgttgccgttctgaaatccgtggtcacccaggcatcttctgcaatcccggtgatgccgctgtatatttctatggcctttaaaatcatgaaagagaagggtatccacgaaggctgtatggagcaagtggaccgcatgatgcgtactcgcctgtacgcggcggacatggcactggatgaccaggcgcgtatccgtatggacgattgggaactgcgtgaagatgttcagcagacttgccgtgatctgtggccgtccattacctccgaaaacctgtgcgagctgaccgattacactggttacaaacaggaatttctgcgtctgttcggtttcggtctggaagaagtagactacgatgcagacgttaacccggacgttaaatttgatgttgtcgaactgtga

SEQ ID NO: 181 - DNA, 1218 bp, Artificial Sequence
  Description of Artificial Sequence Synthetic Eg-ter polynucleotide
atggccatgttcaccactaccgccaaggttattcagccgaaaatccgtggttttatctgtacgaccacccacccgattggctgtgaaaaacgcgtgcaggaagaaattgcttacgcacgtgcacatccaccgaccagcccgggtccgaaacgtgtcctggtcatcggctgttccactggctacggcctgtctactcgtatcaccgcagctttcggctatcaggcggctactctgggcgtgttcctggctggtccgccgactaaaggtcgcccggctgcggccggttggtataacaccgtagctttcgaaaaagcggccctggaagccggtctgtatgcccgctccctgaacggtgacgcttttgactctactaccaaagcacgcaccgtggaagctatcaaacgtgacctgggcaccgttgacctggtggtttatagcattgcagctccgaaacgtaccgatccggctaccggcgtgctgcacaaagcgtgtctgaaaccgatcggtgcgacctacaccaaccgtacggtaaatactgacaaagctgaagttacggacgtgtccatcgaaccggcgagcccagaagaaattgcagacactgtgaaagtaatgggtggcgaagactgggaactgtggattcaggctctgtctgaagccggcgttctggcagaaggcgcgaaaaccgtcgcatactcttatatcggtccggagatgacctggccggtgtactggtccggcaccattggtgaagccaaaaaggatgttgaaaaagccgctaaacgtattacccagcagtacggctgtccggcatacccggttgtggcaaaagcactggtgacgcaggcatcctccgcgatcccggtcgtcccgctgtatatttgtctgctgtaccgtgtaatgaaagaaaaaggcactcacgaaggttgcatcgaacaaatggtgcgtctgctgaccacgaaactgtacccggaaaacggtgccccgatcgttgatgaagcgggccgtgttcgtgtggacgattgggaaatggcagaagacgttcagcaagccgttaaagacctgtggagccaggtgagcacggcaaacctgaaagatatttccgacttcgccggttaccaaaccgagttcctgcgcctgtttggttttggtatcgatggcgtggactatgaccagccggttgacgtagaggcagacctgccgagcgcagctcagcagtaa

SEQ ID NO: 182 - DNA, 1344 bp, Artificial Sequence
  Description of Artificial Sequence Synthetic Sc-ccr polynucleotide
atgaccgtgaaagacattctggacgctattcaatctaaagacgccacttccgcggatttcgcagctctgcaactgccggagtcctaccgtgccatcaccgttcacaaagatgaaactgaaatgttcgcgggtctggaaactcgtgacaaagatccacgtaaatccattcacctggacgaagttccagtgccggaactgggtccgggcgaagccctggtggcagttatggcaagctccgttaactacaactctgtatggacgtctatctttgaaccggtaagcaccttcgccttcctggaacgctacggcaaactgtctccgctgaccaaacgtcatgatctgccataccacatcatcggttctgacctggcaggcgtcgtcctgcgtaccggccctggtgttaacgcctggcagccgggtgacgaagtcgttgcccattgcctgtctgttgaactggaatcccctgatggccatgatgacaccatgctggacccggagcagcgtatttggggcttcgaaactaactttggtggtctggctgagattgctctggtgaagactaaccagctgatgccgaaaccaaaacacctgacttgggaagaagccgcggctccgggcctggtcaacagcactgcgtatcgtcagctggtttctcgtaacggtgctgctatgaaacagggtgataacgttctgatctggggcgcgtccggtggtctgggctcttacgcgacccagttcgcactggccggtggcgcgaatccgatctgcgttgttagctctccgcagaaagctgaaatttgtcgttctatgggcgcagaagcgatcattgatcgcaacgcagagggctacaaattttggaaagacgaacatacccaggaccctaaggaatggaagcgtttcggcaaacgtatccgcgaactgactggtggtgaagacattgatatcgtttttgaacaccctggtcgtgagacttttggtgcgtctgtatacgttacccgcaagggcggtacgatcaccacctgtgcatctacctctggctacatgcatgagtatgataaccgttacctgtggatgtccctgaaacgtatcatcggctctcactttgctaactatcgcgaagcctatgaggcaaaccgtctgatcgctaaaggcaaaattcatccgactctgtctaaaacctattccctggaggaaactggccaggcggcctacgacgtacaccgtaacctgcaccagggcaaagttggcgttctgtgcctggctccggaagaaggtctgggtgttcgtgacgctgaaatgcgtgctcagcacattgacgcgattaaccgtttccgtaatgtgtga

SEQ ID NO: 183 - DNA, 36 bp, Artificial Sequence
  Description of Artificial Sequence Synthetic Gevo-345 primer
atgtttgtcgacatgatagtaaaagcaaagtttgta

SEQ ID NO: 184 - DNA, 41 bp, Artificial Sequence
  Description of Artificial Sequence Synthetic Gevo-346 primer
cttaatgcggccgcttaaggttctaattttcttaataattc

SEQ ID NO: 185 - DNA, 35 bp, Artificial Sequence
  Description of Artificial Sequence Synthetic Gevo-343 primer
gcttgagtcgacatgatcattaaaccgaaagttcg

SEQ ID NO: 186 - DNA, 37 bp, Artificial Sequence
  Description of Artificial Sequence Synthetic Gevo-344 primer
atttaaggatcctcacagttcgacaacatcaaattta

SEQ ID NO: 187 - DNA, 32 bp, Artificial Sequence
  Description of Artificial Sequence Synthetic Gevo-347 primer
catcacgtcgacatggccatgttcaccactac

SEQ ID NO: 188 - DNA, 31 bp, Artificial Sequence
  Description of Artificial Sequence Synthetic Gevo-348 primer
ctcgcgggatccttactgctgagctgcgctc

SEQ ID NO: 189 - DNA, 33 bp, Artificial Sequence
  Description of Artificial Sequence Synthetic Gevo-341 primer
gtcttagtcgacatgaccgtgaaagacattctg

SEQ ID NO: 190 - DNA, 34 bp, Artificial Sequence
  Description of Artificial Sequence Synthetic Gevo-342 primer
attggcggatcctcacacattacggaaacggtta